SEQUENCE LISTING (134 sequences)

SEQ ID NO: 1 - DNA, 18 bp, synthetic construct
  misc_feature 1..18: note = primer
  source 1..18: mol_type = other DNA
cagagttcatcatggata

SEQ ID NO: 2 - DNA, 19 bp, synthetic construct
  misc_feature 1..19: note = primer
  source 1..19: mol_type = other DNA
ctagcatacaaagaaagag

SEQ ID NO: 3 - DNA, 1455 bp, Panax ginseng
  misc_feature 1..1455: note = GT29-32
  source 1..1455: mol_type = other DNA
ggggatcctctagagatatccagagttcatcatggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagtagagcttcatcttccctcttcccctgatcttccccctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggaaatcgcttcgtctcacaatattccggcagtttatttcctaacctcggcagcagccacctcttccatgggcctacatgctttcaaaaactcaggtgaaaaatacccatttccagatttttatgataacagtaatattacccctgaaccaccttctgcagataaaatgaagttatttcatgattttgtcgcttgtttcaaacgatcttgcgacattattttgattaagagttttagagaactggaagggaaatatattgattttctttccactttatctaagaaaactttggttcctgttggtccactcgttcaagatcctatgggacatgatgaagatccaaaaacagggcatcttataaactggcttgacaagagggctgaatctacagtggtgtttgtctgctttggaagtgagtattttccctccaatgaggaattggaagaattagcaattgggctagagattagcatggttagtttcatattggctgtgagatttcctgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttatgagagatgaaaatgggaagtataagagagaagcgattgcagaggtaataagaaaagtcgtgatggagaaaaatggggaggttatgaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaactctttctttgtatgctaggatatcgtcgacc

SEQ ID NO: 4 - protein, 442 aa, Panax ginseng
  REGION 1..442: note = misc_feature - GT29-32
MDNQEGRISIALLPFLAHGHISPFFELAKQLAKRNCNVFLCSTPINLSSIKNKDSSASIKLVELHLPSSPDLPPHYHTTNGLPSHLMVPLRNAFETAAPTFSEILKTLNPDLLIYDFNPSWAPEIASSHNIPAVYFLTSAAATSSMGLHAFKNSGEKYPFPDFYDNSNITPEPPSADKMKLFHDFVACFKRSCDIILIKSFRELEGKYIDFLSTLSKKTLVPVGPLVQDPMGHDEDPKTGHLINWLDKRAESTVVFVCFGSEYFPSNEELEELAIGLEISMVSFILAVRFPEGEKKGILPEGFVQRVGDRGLVVEGWAPQARILGHSSTGGFVSHCGWSSIMESVKFGVPVIAMARHLDQPLNAKLAAEVGVGMEVMRDENGKYKREAIAEVIRKVVMEKNGEVMRRKARELSEKMKEIGEQLIDRAVEELVQICKKKKDEQ

SEQ ID NO: 5 - DNA, 1461 bp, Panax ginseng
  misc_feature 1..1461: note = GT29-33
  source 1..1461: mol_type = other DNA
ccggggatcctctagagatatccagagttcatcatggatatcgaaaaaggtagaatcagtatagttatgctgccatttttagcccatggccacatttctccattctttgagctagccaagcatctctcaaaaagaaattgtaatatattcctctgttctaccccaatcaatcttagctccatcaagaacagagtatctgataaggattcctctgcttctataaaactagtagagcttcatcttccctcttcccctgatcttccccctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggaaatcgcttcgtctcacaatattccggcagtttatttcctaacctcggcagcagccacctcttccatgggcctacatgctttcaaaaactcaggtgaaaaatacccatttccagatttttatgataacagtaatattacccctgaaccaccttctgcagataaaatgaagttatttcatgattttgtcgcttgtttcaaacgatcttgcgacattattttgattaagagttttagagaactggaagggaaatatattgattttctttccactttatctaagaaaactttggttcctgttggtccactcgttcaagatcctatgggacatgatgaagatccaaaaacagggcatcttataaactggcttgacaagagggctgaatctacagtggtgtttgtctgctttggaagtgagtattttccctccaatgaggaattggaagaattagcaattgggctagagattagcatggttagtttcatattggctgtgagatttcctgaaggagagaaaaaagggattttaccagaggggtttgttcaaagggtaggagacagaggattggttgtggaggggtgggctccacagtcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttatgagagatgaaaatgggaagtataagagagaagcgattgcagaggtaataagaaaagtcgtgatggagaaaaatggggaggttatgaggaggaaagcaagggaattgagtgagaaaatgaaagtgaaaggagagcaagagattggtagggcggtggaggagctagtacaaatttgtaagaagaagaagcagcacgcacaatattaatagtactttttaaccctttagtcatttttatgagctaaggtcgagttttgaactctttctttgtatgctaggatatcgtcg

SEQ ID NO: 6 - protein, 448 aa, Panax ginseng
  REGION 1..448: note = misc_feature - GT29-33
MDIEKGRISIVMLPFLAHGHISPFFELAKHLSKRNCNIFLCSTPINLSSIKNRVSDKDSSASIKLVELHLPSSPDLPPHYHTTNGLPSHLMVPLRNAFETAAPTFSEILKTLNPDLLIYDFNPSWAPEIASSHNIPAVYFLTSAAATSSMGLHAFKNSGEKYPFPDFYDNSNITPEPPSADKMKLFHDFVACFKRSCDIILIKSFRELEGKYIDFLSTLSKKTLVPVGPLVQDPMGHDEDPKTGHLINWLDKRAESTVVFVCFGSEYFPSNEELEELAIGLEISMVSFILAVRFPEGEKKGILPEGFVQRVGDRGLVVEGWAPQSRILGHSSTGGFVSHCGWSSIMESVKFGVPVIAMARHLDQPLNAKLAAEVGVGMEVMRDENGKYKREAIAEVIRKVVMEKNGEVMRRKARELSEKMKVKGEQEIGRAVEELVQICKKKKQHAQY

SEQ ID NO: 7 - DNA, 1447 bp, Panax ginseng
  misc_feature 1..1447: note = GT29-34
  source 1..1447: mol_type = other DNA
gggatcctctagagatatccagagttcatcatggatatcgaaaaaggtagaatcattatagttatgctgccatttttagcccatggccacatttctccattctttgagctagccaagcatctctcaaaaagaaattgtaatatattcctctgttctaccccaatcaatcttagctccatcaagaacagagtatctgataaggattcctctgcttctataaaactagtagagcttcatcttccctcttcccctgatcttccccctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggaaatcgcttcgtctcacaatattccggcagtttatttcctaacctcggcagcagccacctcttccatgggcctacatgctttcaaaaactcaggtgaaaaatacccatttccagatttttatgataacagtaatattacccctgaaccaccttctgcagataaaatgaagttatttcatgattttgtcgcttgtttcaaacgatcttgcgacattattttgattaagagttttagagaactggaagggaaatatattgattttctttccactttatctaagaaaactttggttcctgttggtccactcgttcaagatcctatgggacatgatgaagatccaaaaacagggcatcttataaactggcttgacaagagggctgaatctacagtggtgtttgtctgctttggaagtgagtattttccctccaatgaggaattggaagaattagcaattgggctagagattagcatggttagtttcatattggctgtgagatttcctgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttatgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatagtaatagtagtagtactaattttgaatgcgcttaggttgggttttgaactctttctttgtatgctaggatatcgtcg

SEQ ID NO: 8 - protein, 446 aa, Panax ginseng
  REGION 1..446: note = misc_feature - GT29-34
MDIEKGRIIIVMLPFLAHGHISPFFELAKHLSKRNCNIFLCSTPINLSSIKNRVSDKDSSASIKLVELHLPSSPDLPPHYHTTNGLPSHLMVPLRNAFETAAPTFSEILKTLNPDLLIYDFNPSWAPEIASSHNIPAVYFLTSAAATSSMGLHAFKNSGEKYPFPDFYDNSNITPEPPSADKMKLFHDFVACFKRSCDIILIKSFRELEGKYIDFLSTLSKKTLVPVGPLVQDPMGHDEDPKTGHLINWLDKRAESTVVFVCFGSEYFPSNEELEELAIGLEISMVSFILAVRFPEGEKKGILPEGFVQRVGDRGLVVEGWAPQARILGHSSTGGFVSHCGWSSIMESVKFGVPVIAMARHLDQPLNAKLAAEVGVGMEVMRDENGKYKREGIAEVIRKVVVEKSGEVMRRKARELSEKMKEKGEEEIDRAVEELVQICKKKKDAQ

SEQ ID NO: 9 - DNA, 23 bp, synthetic construct
  misc_feature 1..23: note = primer
  source 1..23: mol_type = other DNA
gttcaaagcccaacctaagcgca

SEQ ID NO: 10 - DNA, 26 bp, synthetic construct
  misc_feature 1..26: note = primer
  source 1..26: mol_type = other DNA
acagcaagagagagacacagagttca

SEQ ID NO: 11 - DNA, 1418 bp, Panax ginseng
  misc_feature 1..1418: note = GT29-4
  source 1..1418: mol_type = other DNA
acagcaagagagagacacagagttcattcatggataaccaagaaggtagaatcagtatagcgttgctaccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcatcttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatagtaatagtagtagtactaattttgaatgcgcttaggttgggctttgaac

SEQ ID NO: 12 - protein, 446 aa, Panax ginseng
  REGION 1..446: note = misc_feature - GT29-4
MDNQEGRISIALLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHLDQPLNGKLAAEVGVGMEVVRDENGKYKREGIAEVIRKVVVEKSGEVMRRKARELSEKMKEKGEEEIDRAVEELVQICKKKKDAQ

SEQ ID NO: 13 - DNA, 1434 bp, Panax ginseng
  misc_feature 1..1434: note = GT29-5
  source 1..1434: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatatatcgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaac

SEQ ID NO: 14 - protein, 446 aa, Panax ginseng
  REGION 1..446: note = misc_feature - GT29-5
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEIGEQLIDRAVEELVQICKKKKDEQ

SEQ ID NO: 15 - DNA, 1484 bp, Panax ginseng
  misc_feature 1..1484: note = GT29-7
  source 1..1484: mol_type = other DNA
aatgaaattatacagagagggagagaaacagagttcattcatggataaccaaaaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaacagagtatctgataaggattcctctgcttctataaaactagtagagcttcatcttccatcttcccctgatcttcctcctcactaccacaccacaaatggcctcccttcccatctcatgatcccactcagaaacgcctttgatacagcaggccccaccttctctgaaatccttaaaaccttaaaccctgatttgcttatttatgatttcaatccctcatgggcaccggagatcgcttcgtctcacaatattccggcagtttgtttcataattggtggagcagcctcctcttccatgagcctacatagtttcaaaaacccaggtgaaaaatacccatttctagattttgatgataacagtaatattacccctgaaccaccttcagcagataacatgaagctattaattaattttatgacttgtttcgaacgatcttgcgacattattttgattaagagttttagagaactagaagggaaatattttgattttttttccactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtgttttctctccaatgaggaattggaagaagtagcgattgggctagagattagcatggttaatttcatatgggctgtgagattaattgaaggagagaaaaaaggggttttaccagaggggtttgttcaaagggtaggagacagaggattggttgtggaggagtgggctccacaggcaagaattttaggacattcaagcaccggtgggtttgtgagccattgtgggtggaattctattacggagagtatgaagtttggggttccagtaattgccatggccaggcattttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatcaggaggaaagcaagggaattgagtgagaaaatgaaagagaaaggagagcaagagattgatagggtagtggaggagctagtacaaatttgtaagaagaagaaagatgaacaatagtaataggactactttttttacctttagaatcattttgaatgcgcttaaggttgagtttttaactttttctttgtacttgtactttgtgttgtgaagaaaacca

SEQ ID NO: 16 - protein, 446 aa, Panax ginseng
  REGION 1..446: note = misc_feature - GT29-7
MDNQKGRISIALLPFLAHGHISPFFELAKQLAKRNCNVFLCSTPINLSSIKNRVSDKDSSASIKLVELHLPSSPDLPPHYHTTNGLPSHLMIPLRNAFDTAGPTFSEILKTLNPDLLIYDFNPSWAPEIASSHNIPAVCFIIGGAASSSMSLHSFKNPGEKYPFLDFDDNSNITPEPPSADNMKLLINFMTCFERSCDIILIKSFRELEGKYFDFFSTLSDKTLVPVGPLVQDPMGHNEDPKTEQFINWLDKRAESTVVFVCFGSECFLSNEELEEVAIGLEISMVNFIWAVRLIEGEKKGVLPEGFVQRVGDRGLVVEEWAPQARILGHSSTGGFVSHCGWNSITESMKFGVPVIAMARHFDQPLNGKLAAEVGVGMEVVRDENGKYKREGIAEVIRKVVVEKSGEVIRRKARELSEKMKEKGEQEIDRVVEELVQICKKKKDEQ

SEQ ID NO: 17 - DNA, 1432 bp, Panax ginseng
  misc_feature 1..1432: note = GT29-9
  source 1..1432: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttga

SEQ ID NO: 18 - protein, 446 aa, Panax ginseng
  REGION 1..446: note = misc_feature - GT29-9
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIAESMKFGVPVIAMARHLDQPLNGKLAAEVGVGMEVVRDENGKYKREDIAGVIRKVVVEKSGEVIRRKARELSEKMKEIGEQLIDRAVEELVQICKKKKDEQ

SEQ ID NO: 19 - DNA, 1434 bp, Panax ginseng
  misc_feature 1..1434: note = GT29-11
  source 1..1434: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcatggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaat

SEQ ID NO: 20 - protein, 446 aa, Panax ginseng
  REGION 1..446: note = misc_feature - GT29-11
MDNQKGRISIVMLPFLAHGHISPFFELAKHLSKRNCNIFLCSTPINLSSIKNRISDKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNNTPEPPSADNMKLLHDFIACFERSCDIILIKSFIELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVMRRKARELSEKMKEKGEEEIDRAVEELVQICKKKKDAQ

SEQ ID NO: 21 - DNA, 1434 bp, Panax ginseng
  misc_feature 1..1434: note = GT29-13
  source 1..1434: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtggtcatggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaac

SEQ ID NO: 22 - protein, 446 aa, Panax ginseng
  REGION 1..446: note = misc_feature - GT29-13
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVVMEVVRDENGKYKREDIAGVIRKVVVEKSGEVIRRKARELSEKMKEIGEQLIDRAVEELVQICKKKKDEQ

SEQ ID NO: 23 - DNA, 1422 bp, Panax ginseng
  misc_feature 1..1422: note = GT29-17
  source 1..1422: mol_type = other DNA
acagcaagagagagacacagagttcattcatggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcatggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaac

SEQ ID NO: 24 - protein, 442 aa, Panax ginseng
  REGION 1..442: note = misc_feature - GT29-17
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVGMEVVRDENGKYKREDIAGVIRKVVVEKSGEVIRRKARELSEKMKEIGEQLIDRAVEELVQICKKKKDEQ

SEQ ID NO: 25 - DNA, 1417 bp, Panax ginseng
  misc_feature 1..1417: note = GT29-18
  source 1..1417: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcatggaggttgtgagagatgaaaatgggaagtataagagagaagatattgcaggggtaataagaaaagtcgtggtggagaaaagtggggaggttatgaggaggaaagcaagggaattgagtgagaaaatgaaagagaaaggagaggaagagattgatagggcagtggaggagctagtacaaatttgtaagaagaagaaagatgcacaatagtaatagtagtagtactaattttgaatgcgcttaggttgggcttaatc

SEQ ID NO: 26 - protein, 446 aa, Panax ginseng
  REGION 1..446: note = misc_feature - GT29-18
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVGMEVVRDENGKYKREDIAGVIRKVVVEKSGEVMRRKARELSEKMKEKGEEEIDRAVEELVQICKKKKDAQ

SEQ ID NO: 27 - DNA, 1418 bp, Panax ginseng
  misc_feature 1..1418: note = GT29-24
  source 1..1418: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcatggaggttgtgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatagtaatagtagtagtactaattttgaatgcgcttaggttgggctttgaac

SEQ ID NO: 28 - protein, 446 aa, Panax ginseng
  REGION 1..446: note = misc_feature - GT29-24
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVGMEVVRDENGKYKREGIAEVIRKVVVEKSGEVMRRKARELSEKMKEKGEEEIDRAVEELVQICKKKKDAQ

SEQ ID NO: 29 - DNA, 1434 bp, Panax ginseng
  misc_feature 1..1434: note = GT29-25
  source 1..1434: mol_type = other DNA
acagcaagagagagacacagagttcattcatggataaccaaaagggtagaatcagtatagttatgctgccatttttagcccatggccacatttctccattctttgagctagccaagcatctctcaaaaagaaattgtaatatattcctctgttctaccccaatcaatcttagctccatcaagaacagaatatctgataaggattcctctgcttctataaaactagtagagcttcatcttccatcttcccctgatcttcctcctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggagatcgcttcgtctcacaatattccggcagtttgtttcataattgggggagcagcctccttttccatgagcctacatagtttcaaaaacccaggtgaaaaatacccatttctagattttgatgataacagtaatattacccctgaaccaccttcagcagataacatgaagttattacttgattttatgacttgtttcgaacgatcttgcgacattattttgattaagagttttagagaactagaagggaaatattttgatttttattctactttatctgataaaactttggttcctgttggtccactcgttcaagatcctatgggccataatgaagatccaaaaacagagcagtttataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcacggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatagtagtaatagactaatttttttccctttaaaatcattttgaatgcgcttaggttgggctttgaac

SEQ ID NO: 30 - protein, 446 aa, Panax ginseng
  REGION 1..446: note = misc_feature - GT29-25
MDNQKGRISIVMLPFLAHGHISPFFELAKHLSKRNCNIFLCSTPINLSSIKNRISDKDSSASIKLVELHLPSSPDLPPHYHTTNGLPSHLMVPLRNAFETAAPTFSEILKTLNPDLLIYDFNPSWAPEIASSHNIPAVCFIIGGAASFSMSLHSFKNPGEKYPFLDFDDNSNITPEPPSADNMKLLLDFMTCFERSCDIILIKSFRELEGKYFDFYSTLSDKTLVPVGPLVQDPMGHNEDPKTEQFINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVGTEVVRDENGKYKREDIAGVIRKVVVEKSGEVIRRKARELSEKMKEIGEQLIDRAVEELVQICKKKKDEQ

SEQ ID NO: 31 - DNA, 43 bp, synthetic construct
  misc_feature 1..43: note = primer
  source 1..43: mol_type = other DNA
taagaaggagatataccatggataaccaagaaggtagaatcag

SEQ ID NO: 32 - DNA, 44 bp, synthetic construct
  misc_feature 1..44: note = primer
  source 1..44: mol_type = other DNA
tgcggccgcaagcttgtcgacttgttcatctttcttcttcttac

SEQ ID NO: 33 - DNA, 42 bp, synthetic construct
  misc_feature 1..42: note = primer
  source 1..42: mol_type = other DNA
taagaaggagatataccatggataaccaaaagggtagaatca

SEQ ID NO: 34 - DNA, 42 bp, synthetic construct
  misc_feature 1..42: note = primer
  source 1..42: mol_type = other DNA
tgcggccgcaagcttgtcgacttgtgcatctttcttcttctt

SEQ ID NO: 35 - DNA, 43 bp, synthetic construct
  misc_feature 1..43: note = primer
  source 1..43: mol_type = other DNA
taagaaggagatataccatggataaccaaaaaggtagaatcag

SEQ ID NO: 36 - DNA, 43 bp, synthetic construct
  misc_feature 1..43: note = primer
  source 1..43: mol_type = other DNA
taagaaggagatataccatggatatcgaaaaaggtagaatcag

SEQ ID NO: 37 - DNA, 39 bp, synthetic construct
  misc_feature 1..39: note = primer
  source 1..39: mol_type = other DNA
tgcggccgcaagcttgtcgacatattgtgcgtgctgctt

SEQ ID NO: 38 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagagcagagtatctgataaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgaagaaagacgcacaatattaa

SEQ ID NO: 39 - protein, 447 aa, Panax notoginseng
MDIEKGRISIVMLPFLAHGHISPFFELAKHLSKRNCNIFLCSTPINLSSIKSRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMKKDAQY

SEQ ID NO: 40 - DNA, 1329 bp, Panax notoginseng
  source 1..1329: mol_type = other DNA
atggataaccaaaaaggtagaatcagtatagcgttgctaccatttttagcccatggtcacatatctcccttctttgagctagccaaacaactggcaaaaagaaattgcaatgttttcctctgttctaccccaatcaatcttagctccatcaagaataaggattcctctgcttctgtaaaactagttgagcttcatcttccatcttcccctgatcttcctcctcactatcacaccacaaatggcctcccttcccatctcatggtcccactcagaaacgcctttgaaacagtaggccccaccttctctgaaatccttaaaaccttaaaccctgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgatattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattgcggagagtatgaggtttggggttccagtaattgccatggctaggcatcttgatcagcctttgaatgctaagctggcggcggaggttggtgtgggcatggaggttgtaagagatgataatgggaaatataagagggaagggattgcagaggtaataagaaaagtcgttgtggagaaaagtggggaggttatcaggaggaaagcaagggagttgagtgagaaaatgaaagagaaaggagagcaagagattgatagggcagtggaggagctagtacaaatttgtaagaagaagaaagatgcacaatag

SEQ ID NO: 41 - protein, 442 aa, Panax notoginseng
MDNQKGRISIALLPFLAHGHISPFFELAKQLAKRNCNVFLCSTPINLSSIKNKDSSASVKLVELHLPSSPDLPPHYHTTNGLPSHLMVPLRNAFETVGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIAESMRFGVPVIAMARHLDQPLNAKLAAEVGVGMEVVRDDNGKYKREGIAEVIRKVVVEKSGEVIRRKARELSEKMKEKGEQEIDRAVEELVQICKKKKDAQ

SEQ ID NO: 42 - DNA, 1344 bp, Panax notoginseng
  source 1..1344: mol_type = other DNA
atggatatcgagaaaggtagaatcagtatagttatgctaccatttttagcccatggtcacatatctccattctttgagctagccaagcatctctcaaaaagaaattgcaatatattcctctgttctaccccaatcaatcttagctccatcaagaacagagtatctgataaggattcctctgcttcaataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgtgagatttcttgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggtcggtgtgggcatggaggttgtgagagatgaaaatgggaagtataagagagaagcgattgcagaggtaataagaaaagtcgtgatggagaaaaatggggaggttatcaggaggaaagcaagggaattgagtgagaaaatgaaagagacaggagagcaagagattggtagggcagtggaggagctagtacaaatttgtaagatgaagaaagacgcacaatattaa

SEQ ID NO: 43 - protein, 447 aa, synthetic construct
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYKREAIAEVIRKVVMEKNGEVIRRKARELSEKMKETGEQEIGRAVEELVQICKMKKDAQY

SEQ ID NO: 44 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagaacagagtatctgataaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgaagaaagacgcacaatattaa

SEQ ID NO: 45 - protein, 447 aa, Panax notoginseng
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMKKDAQY

SEQ ID NO: 46 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagaacagagtatctgataaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgatgaaagacgcacaatattaa

SEQ ID NO: 47 - protein, 447 aa, Panax notoginseng
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMMKDAQY

SEQ ID NO: 48 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagaacagagtatctggtaaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgaagaaagacgcacaatattaa

SEQ ID NO: 49 - protein, 447 aa, Panax notoginseng
MDIEKGRISIVMLPFLAHGHISPFFELAKHLSKRNCNIFLCSTPINLSSIKNRVSGKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMKKDAQY

SEQ ID NO: 50 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagaacagagtatctgataaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgtttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgaagaaagacgcacaatattaa

SEQ ID NO: 51 - protein, 447 aa, Panax notoginseng
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLFIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMKKDAQY

SEQ ID NO: 52 - DNA, 1338 bp, Panax notoginseng
  source 1..1338: mol_type = other DNA
atcgagaaaggtagaatcagtatagttatgctaccatttttagcccatggtcacatatctccattctttgagctagccaagcatctctcaaaaagaaattgcaatatattcctctgttctaccccaatcaatcttagctccatcaagaacagagtatctgataaggattcctctgcttcaataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgtgagatttcttgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcgggggtcggtgtgggcatggaggttgtgagagatgaaaatgggaagtataagagagaagcgattgcagaggtaataagaaaagtcgtgatggagaaaaatggggaggttatcaggaggaaagcaagggaattgagtgagaaaatgaaagagacaggagagcaagagattggtagggcagtggaggagctagtacaaatttgtaagatgaagaaagacgcacaatattaa

SEQ ID NO: 53 - protein, 447 aa, Panax notoginseng
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAGVGVGMEVVRDENGKYKREAIAEVIRKVVMEKNGEVIRRKARELSEKMKETGEQEIGRAVEELVQICKMKKDAQY

SEQ ID NO: 54 - DNA, 1344 bp, Panax notoginseng
  source 1..1344: mol_type = other DNA
atggatatcgagaaaggtagaatcagtatagttatgctaccatttttagcccatggtcacatatctccattccttgagctagccaagcatctctcaaaaagaaattgcaatatattcctctgttctaccccaatcaatcttagctccatcaagaacagagtatctgataaggattcctctgcttcaataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgtgagatttcttgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggtcggtgtgggcatggaggttgtgagagatgaaaatgggaagtataagagagaagcgattgcagaggtaataagaaaagtcgtgatggagaaaaatggggaggttatcaggaggaaagcaagggaattgagtgagaaaatgaaagagacaggagagcaagagattggtagggcagtggaggagctagtacaaatttgtaagatgaagaaagacgcacaatattaa

SEQ ID NO: 55 - protein, 447 aa, Panax notoginseng
MDIEKGRISIVMLPFLAHGHISPFLELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYKREAIAEVIRKVVMEKNGEVIRRKARELSEKMKETGEQEIGRAVEELVQICKMKKDAQY

SEQ ID NO: 56 - DNA, 1344 bp, Panax notoginseng
  source 1..1344: mol_type = other DNA
atgtatatcgagaaaggtagaatcagtatagttatgctaccatttttagcccatggtcacatatctccattctttgagctagccaagcatctctcaaaaagaaattgcaatatattcctctgttctaccccaatcaatcttagctccatcaagaacagagtatctgataaggattcctctgcttcaataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcctttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgtgagatttcttgaaggagagaaaaaaggggttttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggtcggtgtgggcatggaggttgtgagagatgaaaatgggaagtataagagagaagcgattgcagaggtaataagaaaagtcgtgatggagaaaaatggggaggttatcaggaggaaagcaagggaattgagtgagaaaatgaaagagacaggagagcaagagattggtagggcagtggaggagctagtacaaatttgtaagatgaagaaagacgcacaatattaa

SEQ ID NO: 57 - protein, 447 aa, Panax notoginseng
MYIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDPLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYKREAIAEVIRKVVMEKNGEVIRRKARELSEKMKETGEQEIGRAVEELVQICKMKKDAQY

SEQ ID NO: 58 - DNA, 1344 bp, Panax notoginseng
  source 1..1344: mol_type = other DNA
atggatatcgaaaaaggtagaatcagtatagttatgctgccatttttagcccatggtcacatatctccattctttgagctagccaagcatctctcaaaaagaaattgcaatatattcctctgttctaccccaatcaatcttagctccatcaagaacagagtatctgataaggattcctctgcttctataaaactagtagagcttcatcttccatcttcccctgatcttcctcctcagtaccacaccacaaatggcctcccttcccatctcatggtcccactcaaaaacgcctttgaaacagtaggccccaccttctctgaaatccttaaaaccttagaccctgatttgcttatttatgatttcaatccctcatgggcaccggagatcgctttgtctcacaatattccggcagtttatttcctaacctcggcagcagccacctcttccgtggccctacgtgctttgaaaaacccaggtgaaaaatacccatttccagatttttatgataacagtaatattacccctgaaccaccttctgcagataaaatgaagctatttcatgattttgttgcttgtttcaaacgatcttgcgacattattttgattaagagttttagagaactagaagggaaatatattgatttgctttccactttatctaagaaaactttggttcctgttggtccactcgttcaagatcatttgggacatgatgaagatccaaaaacagggcatcttataaactggcttgacaaaagggctgaatctacagtggtgtttgtctgctttggaagtgagtattttccctccaatgaggaattggaagaagtagcaattgggctagagattagcatggttaatttcatattggctgttagatttcttgaaggagagaaaaaaggggttttaccagaagggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttgtgagagatgaaaatgggaagtatacgagagaagcgattgcagaggtaataagaaaagttgtgatggagaaaaatggggaggttatcaggaggaaagcaagggaattgagtgataaaatgaaagagaaaggagagcaagagattggtagggcagtggaggagctagtacaaatttgtaagatgaagaaagacgcacaatattaa

SEQ ID NO: 59 - protein, 447 aa, Panax notoginseng
MDIEKGRISIVMLPFLAHGHISPFFELAKHLSKRNCNIFLCSTPINLSSIKNRVSDKDSSASIKLVELHLPSSPDLPPQYHTTNGLPSHLMVPLKNAFETVGPTFSEILKTLDPDLLIYDFNPSWAPEIALSHNIPAVYFLTSAAATSSVALRALKNPGEKYPFPDFYDNSNITPEPPSADKMKLFHDFVACFKRSCDIILIKSFRELEGKYIDLLSTLSKKTLVPVGPLVQDHLGHDEDPKTGHLINWLDKRAESTVVFVCFGSEYFPSNEELEEVAIGLEISMVNFILAVRFLEGEKKGVLPEGFVQRVGDRGLVVEGWAPQARILGHSSTGGFVSHCGWSSIMESVKFGVPVIAMARHLDQPLNAKLAAEVGVGMEVVRDENGKYTREAIAEVIRKVVMEKNGEVIRRKARELSDKMKEKGEQEIGRAVEELVQICKMKKDAQY

SEQ ID NO: 60 - DNA, 41 bp, synthetic construct
  misc_feature 1..41: note = primer
  source 1..41: mol_type = other DNA
taagaaggagatataccatggatatcgaaaaaggtagaatc

SEQ ID NO: 61 - DNA, 44 bp, synthetic construct
  misc_feature 1..44: note = primer
  source 1..44: mol_type = other DNA
tgcggccgcaagcttgtcgacatattgtgcgtctttcttcatct

SEQ ID NO: 62 - DNA, 46 bp, synthetic construct
  misc_feature 1..46: note = primer
  source 1..46: mol_type = other DNA
taagaaggagatataccatgggtgataaccaaaaaggtagaatcag

SEQ ID NO: 63 - DNA, 44 bp, synthetic construct
  misc_feature 1..44: note = primer
  source 1..44: mol_type = other DNA
tgcggccgcaagcttgtcgacttgtgcatctttcttcttcttac

SEQ ID NO: 64 - DNA, 45 bp, synthetic construct
  misc_feature 1..45: note = primer
  source 1..45: mol_type = other DNA
taagaaggagatataccatgggtgatatcgagaaaggtagaatca

SEQ ID NO: 65 - DNA, 41 bp, synthetic construct
  misc_feature 1..41: note = primer
  source 1..41: mol_type = other DNA
tgcggccgcaagcttgtcgacatattgtgcgtctttcttca

SEQ ID NO: 66 - DNA, 45 bp, synthetic construct
  misc_feature 1..45: note = primer
  source 1..45: mol_type = other DNA
taagaaggagatataccatgggtgatatcgaaaaaggtagaatca

SEQ ID NO: 67 - DNA, 41 bp, synthetic construct; identical to SEQ ID NO: 65
  misc_feature 1..41: note = primer
  source 1..41: mol_type = other DNA

SEQ ID NO: 68 - DNA, 45 bp, synthetic construct; identical to SEQ ID NO: 66
  misc_feature 1..45: note = primer
  source 1..45: mol_type = other DNA

SEQ ID NO: 69 - DNA, 42 bp, synthetic construct
  misc_feature 1..42: note = primer
  source 1..42: mol_type = other DNA
tgcggccgcaagcttgtcgacatattgtgcgtctttcatcat

SEQ ID NO: 70 - DNA, 47 bp, synthetic construct
  misc_feature 1..47: note = primer
  source 1..47: mol_type = other DNA
taagaaggagatataccatgggtgatatcgaaaaaggtagaatcagt

SEQ ID NO: 71 - DNA, 44 bp, synthetic construct
  misc_feature 1..44: note = primer
  source 1..44: mol_type = other DNA
gtgcggccgcaagcttgtcgacatattgtgcgtctttcttcatc

SEQ ID NO: 72 - DNA, 46 bp, synthetic construct
  misc_feature 1..46: note = primer
  source 1..46: mol_type = other DNA
taagaaggagatataccatgggtgatatcgaaaaaggtagaatcag

SEQ ID NO: 73 - DNA, 43 bp, synthetic construct
  misc_feature 1..43: note = primer
  source 1..43: mol_type = other DNA
gtgcggccgcaagcttgtcgacatattgtgcgtctttcttcat

SEQ ID NO: 74 - DNA, 44 bp, synthetic construct
  misc_feature 1..44: note = primer
  source 1..44: mol_type = other DNA
taagaaggagatataccatgggtgatatcgagaaaggtagaatc

SEQ ID NO: 75 - DNA, 42 bp, synthetic construct
  misc_feature 1..42: note = primer
  source 1..42: mol_type = other DNA
gtgcggccgcaagcttgtcgacatattgtgcgtctttcttca

SEQ ID NO: 76 - DNA, 44 bp, synthetic construct; identical to SEQ ID NO: 74
  misc_feature 1..44: note = primer
  source 1..44: mol_type = other DNA

SEQ ID NO: 77 - DNA, 42 bp, synthetic construct; identical to SEQ ID NO: 75
  misc_feature 1..42: note = primer
  source 1..42: mol_type = other DNA

SEQ ID NO: 78 - DNA, 38 bp, synthetic construct
  misc_feature 1..38: note = primer
  source 1..38: mol_type = other DNA
taagaaggagatataccatgtatatcgagaaaggtaga

SEQ ID NO: 79 - DNA, 41 bp, synthetic construct; identical to SEQ ID NO: 65
  misc_feature 1..41: note = primer
  source 1..41: mol_type = other DNA

SEQ ID NO: 80 - DNA, 40 bp, synthetic construct
  misc_feature 1..40: note = primer
  source 1..40: mol_type = other DNA
taagaaggagatataccatggatatcgaaaaaggtagaat

SEQ ID NO: 81 - DNA, 45 bp, synthetic construct
  misc_feature 1..45: note = primer
  source 1..45: mol_type = other DNA
tgcggccgcaagcttgtcgacatattgtgcgtctttcttcatctt

SEQ ID NO: 82 - DNA, 22 bp, synthetic construct
  misc_feature 1..22: note = primer
  source 1..22: mol_type = other DNA
agagttcattcatggataacca

SEQ ID NO: 83 - DNA, 22 bp, synthetic construct
  misc_feature 1..22: note = primer
  source 1..22: mol_type = other DNA
ttaagcgcattcaaaaatattc

SEQ ID NO: 84 - DNA, 21 bp, synthetic construct
  misc_feature 1..21: note = primer
  source 1..21: mol_type = other DNA
agagttcatcatggatatcga

SEQ ID NO: 85 - DNA, 22 bp, synthetic construct
  misc_feature 1..22: note = primer
  source 1..22: mol_type = other DNA
catagctcattcaaaatgactc

SEQ ID NO: 86 - DNA, 21 bp, synthetic construct
  misc_feature 1..21: note = primer
  source 1..21: mol_type = other DNA
catagctcattcaaaatgaat

SEQ ID NO: 87 - DNA, 34 bp, synthetic construct
  misc_feature 1..34: note = primer
  source 1..34: mol_type = other DNA
tatctatcgtatacgccagagttcatcatggata

SEQ ID NO: 88 - DNA, 35 bp, synthetic construct
  misc_feature 1..35: note = primer
  source 1..35: mol_type = other DNA
tgcagtcgagatacatctagcatacaaagaaagag

SEQ ID NO: 89 - DNA, 1329 bp, Panax ginseng
  source 1..1329: mol_type = other DNA
atggataaccaaaaaggtagaatcagtatagcgttgctaccatttttagcccatggtcacatatctcccttctttgagctagccaaacaactcgcgaaaagaaattgcaatgttttcctctgttctaccccaatcaatcttagctccatcaaggataaggattcctctgcttctataaaactagttgagcttcatcttccatcttcccctgatcttcctcctcactatcacaccacaaatggcctcccttcccatctcatgctcccactcagaaacgcctttgaaactgcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatag

SEQ ID NO: 90 - protein, 442 aa, Panax ginseng
MDNQKGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVMRRKARELSEKMKEKGEEEIDRAVEELVQICKKKKDAQ

SEQ ID NO: 91 - DNA, 1329 bp, Panax ginseng
  source 1..1329: mol_type = other DNA
atggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcacggttaatttcatatgggctgtgagattaattgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgagaatgggaagtataagagagaagggattgcagaggtaataagaaaagtggttgtggagaaaagtggggaggttatcaggaggaaagcaagggagttgagtgagaaaatgaaagagaaaggagagcaagagattgatagggcattggaggagctagtacaaatttgtaagaagaagaaagatgaacaatag

SEQ ID NO: 92 - protein, 442 aa, Panax ginseng
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 93 - DNA, 1332 bp, Panax ginseng
  source 1..1332: mol_type = other DNA
atggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaataatacccctgaaccaccttctgcagataacatgaagctacttcatgattttatcgcttgtttcgaacgatcttgcgacattattttgattaagagttttatagaactagaagggaaatatatcgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttatgagagatgaaaatgggaagtataagagagaagcgattgcagaggtaataagaaaagtcgtgatggagaaaaatggggaggttatgaggaggaaagcaagggaattgagtgagaaaatgaaagtgaaaggagagcaagagattggtagggcggtggaggagctagtacaaatttgtaagaagaagaagcagcacgcacaatat

SEQ ID NO: 94 - protein, 444 aa, Panax ginseng
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNNTPEPPSADNMKLLHDFIACFERSCDIILIKSFIELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSIMESVKFGVPVIAMARHLDQPLNAKLAAEVGVGMEVMRDENGKYKREAIAEVIRKVVMEKNGEVMRRKARELSEKMKVKGEQEIGRAVEELVQICKKKKQHAQY

SEQ ID NO: 95 - DNA, 1329 bp, Panax ginseng
  source 1..1329: mol_type = other DNA
atggataaccaaaaaggtagaatcagtatagcgttgctaccatttttagcccatggtcacatatctcccttctttgagctagccaaacaactcgcgaaaagaaattgcaatgttttcctctgttctaccccaatcaatcttagctccatcaaggataaggattcctctgcttctataaaactagttgagcttcatcttccatcttcccctgatcttcctcctcactatcacaccacaaatggcctcccttcccatctcatgctcccactcagaaacgcctttgaaactgcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaatggctgaatctacagtggtgtttgtctgctttggaagtgagtattttctctccaatgaggaattggaagaagtagcaattgggctagagattagcacggttaatttcatatgggctgtgagattaattgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgagaatgggaagtataagagagaagggattgcagaggtaataagaaaagtggttgtggagaaaagtggggaggttatcaggaggaaagcaagggagttgagtgagaaaatgaaagagaaaggagagcaagagattgatagggcattggaggagctagtacaaatttgtaagaagaagaaagatgaacaatag

SEQ ID NO: 96 - protein, 442 aa, Panax ginseng
MDNQKGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKMAESTVVFVCFGSEYFLSNEELEEVAIGLEISTVNFIWAVRLIEGEKKGILPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 97 - DNA, 1347 bp, Panax ginseng
  source 1..1347: mol_type = other DNA
atggatatcgaaaaaggtagaatcagtatagttatgctgccatttttagcccatggccacatttctccattctttgagctagccaagcatctctcaaaaagaaattgtaatatattcctctgttctaccccaatcaatcttagctccatcaagaacagagtatctgataaggattcctctgcttctataaaactagtagagcttcatcttccctcttcccctgatcttccccctcactaccacaccacaaatggcctcccttcccatctcatggtcccactcagaaacgcctttgaaacagcagcccccaccttctctgaaatccttaaaaccttaaaccctgatttgcttatttatgatttcaatccctcatgggcaccggaaatcgcttcgtctcacaatattccggcagtttatttcctaacctcggcagcagccacctcttccatgggcctacatgctttcaaaaactcaggtgaaaaatacccatttccagatttttatgataacagtaatattacccctgaaccaccttctgcagataaaatgaagctatttcatgattttgtcgcttgtttcaaacgatcttgcgacattattttgattaagagttttagagaactggaagggaaatatattgattttctttccactttatctaagaaaactttggttcctgttggtccactcgttcaagatcctatgggacatgatgaagatccaaaaacagggcatcttataaactggcttgacaagagggctgaatctacagtggtgtttgtctgctttggaagtgagtattttccctccaatgaggaattggaagaattagcaattgggctagagattagcatggttagtttcatattggctgtgagatttcctgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattatggagagtgtgaagtttggggttccagtaattgccatggccaggcatcttgatcagcctttgaatgctaagctggcggcggaggttggtgtgggcatggaggttatgagagatgaaaatgggaagtataagagagaagcgattgcagaggtaataagaaaagtcgtgatggagaaaaatggggaggttatgaggaggaaagcaagggaattgagtgagaaaatgaaagtgaaaggagagcaagagattggtagggcggtggaggagctagtacaaatttgtaagaagaagaagcagcacgcacaatattaa

SEQ ID NO: 98 - protein, 448 aa, Panax ginseng
MDIEKGRISIVMLPFLAHGHISPFFELAKHLSKRNCNIFLCSTPINLSSIKNRVSDKDSSASIKLVELHLPSSPDLPPHYHTTNGLPSHLMVPLRNAFETAAPTFSEILKTLNPDLLIYDFNPSWAPEIASSHNIPAVYFLTSAAATSSMGLHAFKNSGEKYPFPDFYDNSNITPEPPSADKMKLFHDFVACFKRSCDIILIKSFRELEGKYIDFLSTLSKKTLVPVGPLVQDPMGHDEDPKTGHLINWLDKRAESTVVFVCFGSEYFPSNEELEELAIGLEISMVSFILAVRFPEGEKKGILPEGFVQRVGDRGLVVEGWAPQARILGHSSTGGFVSHCGWSSIMESVKFGVPVIAMARHLDQPLNAKLAAEVGVGMEVMRDENGKYKREAIAEVIRKVVMEKNGEVMRRKARELSEKMKVKGEQEIGRAVEELVQICKKKKQHAQY

SEQ ID NO: 99 - DNA, 1329 bp, Panax ginseng
  source 1..1329: mol_type = other DNA
atggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaataatacccctgaaccaccttctgcagataacatgaagctacttcatgattttatcgcttgtttcgaacgatcttgcgacattattttgattaagagttttatagaactagaagggaaatatatcgatttgctttccactttatctgataaaactttggttcctgttggtccactcgttcaagatcctatgggccataatgaagatccaaaaacagagcagattataaactggcttgacaaaagggctgaatctacagtggtgtttgtctactttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatag

SEQ ID NO: 100 - protein, 442 aa, synthetic construct
  REGION 1..442: note = primer
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNNTPEPPSADNMKLLHDFIACFERSCDIILIKSFIELEGKYIDLLSTLSDKTLVPVGPLVQDPMGHNEDPKTEQIINWLDKRAESTVVFVYFGSEYFLSNEELEEVAIGLEISMVNFIWAVRLIEGEKKGVLPEGFVQRVGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVMRRKARELSEKMKEKGEEEIDRAVEELVQICKKKKDAQ

SEQ ID NO: 101 - DNA, 48 bp, synthetic construct
  misc_feature 1..48: note = primer
  source 1..48: mol_type = other DNA
aactttaagaaggagatataccatgggcatggataaccaaaaaggtag

SEQ ID NO: 102 - DNA, 46 bp, synthetic construct
  misc_feature 1..46: note = primer
  source 1..46: mol_type = other DNA
tcgagtgcggccgcaagcttgtcgacctattgtgcatctttcttct

SEQ ID NO: 103 - DNA, 48 bp, synthetic construct
  misc_feature 1..48: note = primer
  source 1..48: mol_type = other DNA
aactttaagaaggagatataccatgggcatggataaccaagaaggtag

SEQ ID NO: 104 - DNA, 46 bp, synthetic construct
  misc_feature 1..46: note = primer
  source 1..46: mol_type = other DNA
tcgagtgcggccgcaagcttgtcgacctattgttcatctttcttct

SEQ ID NO: 105 - DNA, 48 bp, synthetic construct; identical to SEQ ID NO: 103
  misc_feature 1..48: note = primer
  source 1..48: mol_type = other DNA

SEQ ID NO: 106 - DNA, 46 bp, synthetic construct
  misc_feature 1..46: note = primer
  source 1..46: mol_type = other DNA
tcgagtgcggccgcaagcttgtcgacttaatattgtgcgtgctgct

SEQ ID NO: 107 - DNA, 48 bp, synthetic construct; identical to SEQ ID NO: 101
  misc_feature 1..48: note = primer
  source 1..48: mol_type = other DNA

SEQ ID NO: 108 - DNA, 46 bp, synthetic construct; identical to SEQ ID NO: 104
  misc_feature 1..46: note = primer
  source 1..46: mol_type = other DNA

SEQ ID NO: 109 - DNA, 49 bp, synthetic construct
  misc_feature 1..49: note = primer
  source 1..49: mol_type = other DNA
aactttaagaaggagatataccatgggcatggatatcgaaaaaggtaga

SEQ ID NO: 110 - DNA, 45 bp, synthetic construct
  misc_feature 1..45: note = primer
  source 1..45: mol_type = other DNA
tcgagtgcggccgcaagcttgtcgacttaatattgtgcgtgctgc

SEQ ID NO: 111 - DNA, 49 bp, synthetic construct
  misc_feature 1..49: note = primer
  source 1..49: mol_type = other DNA
aactttaagaaggagatataccatgggcatggataaccaagaaggtaga

SEQ ID NO: 112 - DNA, 48 bp, synthetic construct
  misc_feature 1..48: note = primer
  source 1..48: mol_type = other DNA
tcgagtgcggccgcaagcttgtcgacctattgtgcatctttcttcttc

SEQ ID NO: 113 - DNA, 26 bp, synthetic construct
  misc_feature 1..26: note = primer
  source 1..26: mol_type = other DNA
agtaagaaaaacagagttcatcatgg

SEQ ID NO: 114 - DNA, 20 bp, synthetic construct
  misc_feature 1..20: note = primer
  source 1..20: mol_type = other DNA
gcctcggttaggctagctgt

SEQ ID NO: 115 - DNA, 1329 bp, Panax ginseng
  misc_feature 1..1329: note = GT29-19
  source 1..1329: mol_type = other DNA
atggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaataatacccctgaaccaccttctgcagataacatgaagctacttcatgattttatcgcttgtttcgaacgatcttgcgacattattttgattaagagttttatagaactagaagggaaatatatcgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacaggcaagaattttaggacattcaagcaccggtgggtttgtgagccattgtgggtggagttctattacggagagtatgaagtttggggttccagtaattgccatggccaggcattttgatcagcctttgaatgctaagctggcggcggaggttggtgtgggcatggaggttgtgagagatgaaaatgggaagtataagagagaagatattgcaggggtaataagaaaagtcgtggtggagaaaagtggggaggttatcaggaggaaagcaagggaattgagtgagaaaatgaaagagataggagagcaattgattgatagggcagtggaggagctagtacaaatttgtaagaagaagaaagatgaacaatag

SEQ ID NO: 116 - protein, 442 aa, Panax ginseng
  REGION 1..442: note = misc_feature - GT29-19
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNNTPEPPSADNMKLLHDFIACFERSCDIILIKSFIELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPQARILGHSSTGGFVSHCGWSSITESMKFGVPVIAMARHFDQPLNAKLAAEVGVGMEVVRDENGKYKREDIAGVIRKVVVEKSGEVIRRKARELSEKMKEIGEQLIDRAVEELVQICKKKKDEQ

SEQ ID NO: 117 - DNA, 1329 bp, Panax ginseng
  misc_feature 1..1329: note = GT29-20
  source 1..1329: mol_type = other DNA
atggataaccaaaaaggtagaatcagtatagcgttgctaccatttttagcccatggtcacatatctcccttctttgagctagccaaacaactcgcgaaaagaaattgcaatgttttcctctgttctaccccaatcaatcttagctccatcaaggataaggattcctctgcttctataaaactagttgagcttcatcttccatcttcccctgatcttcctcctcactatcacaccacaaatggcctcccttcccatctcatgctcccactcagaaacgcctttgaaactgcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcacggttaatttcatatgggctgtgagattaattgaaggagagaaaaaagggattttaccagaggggtttgttcaaagggcaggagacagaggattggttgtggaggggtgggctccacaggcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgagaatgggaagtataagagagaagggattgcagaggtaataagaaaagtggttgtggagaaaagtggggaggttatcaggaggaaagcaagggagttgagtgagaaaatgaaagagaaaggagagcaagagattgatagggcattggaggagctagtacaaatttgtaagaagaagaaagatgaacaatag

SEQ ID NO: 118 - protein, 442 aa, Panax ginseng
  REGION 1..442: note = misc_feature - GT29-20
MDNQKGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRAGDRGLVVEGWAPQARILGHSSTGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 119 - DNA, 1329 bp, Panax ginseng
  misc_feature 1..1329: note = GT29-21
  source 1..1329: mol_type = other DNA
atggataaccaaaaaggtagaatcagtatagcgttgctaccatttttagccaatggtcacatatctcccttctttgagctagccaaacaactcgcgaaaagaaattgcaatgttttcctctgttctaccccaatcaatcttagctccatcaaggataaggattcctctgcttctataaaactagttgagcttcatcttccatcttcccctgatcttcctcctcactatcacaccacaaatggcctcccttcccatctcatgctcccactcagaaacgcctttgaaactgcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcacggttaatttcatatgggctgtgagattaattgaaggagagaaaaaagggattttaccagaggggtttgttcaaagggtaggagacagaggattggttgtggaggggtgggctccacaggcaagaattttaggacattcaagcatcggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgagaatgggaagtataagagagaagggattgcagaggtaataagaaaagtggttgtggagaaaagtggggaggttatcaggaggaaagcaagggagttgagtgagaaaatgaaagagaaaggagagcaagagattgatagggcattggaggagctagtacaaatttgtaagaagaagaaagatgaacaatag

SEQ ID NO: 120 - protein, 442 aa, Panax ginseng
  REGION 1..442: note = misc_feature - GT29-21
MDNQKGRISIALLPFLANGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSIGGFVSHCGWSSIAESMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 121 - DNA, 1329 bp, Panax ginseng
  misc_feature 1..1329: note = GT29-22
  source 1..1329: mol_type = other DNA
atggataaccaaaaaggtagaatcagtatagcgttgctaccatttttagcccatggtcacatatctcccttctttgagctagccaaacaactcgcgaaaagaaattgcaatgttttcctctgttctaccccaatcaatcttagctccatcaaggataaggattcctctgcttctataaaactagttgagcttcatcttccatcttcccctgatcttcctcctcactatcacaccacaaatggcctcccttcccatctcatgctcccactcagaaacgcctttgaaactgcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaatattacccctgaaccaccttctgcagataacatgaagctacttcatgattttatcgcttgtttcgaacgatcttgcgacattattttgattaagagttttagagaactagaagggaaatatattgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcacggttaatttcatatgggctgtgagattaattgaaggagagaaaaaagggattttaccagaggggtttgttcaaagggtaggagacagaggattggttgtggaggggtgggctccacaggcaagaattttaggacattcaagcaccggtgggtttgtgagccattgtgggtggagttctattgcggagattatgaagtttggggttccagtaattgccatggccaggcatcttgatcagcctttgaatggtaagctggcggcggaggttggtgtgggcatggaggttgtgagagatgagaatgggaagtataagagagaagggattgcagaggtaataagaaaagtggttgtggagaaaagtggggaggttatcaggaggaaagcaagggagttgagtgagaaaatgaaagagaaaggagagcaagagattgatagggcattggaggagctagtacaaatttgtaagaagaagaaagatgaacaatag

SEQ ID NO: 122 - protein, 442 aa, Panax ginseng
  REGION 1..442: note = misc_feature - GT29-22
MDNQKGRISIALLPFLAHGHISPFFELAKQLAKRNCNVFLCSTPINLSSIKDKDSSASIKLVELHLPSSPDLPPHYHTTNGLPSHLMLPLRNAFETAGPTFSEILKTLNPDLLIYDFNPSWAPEIASSHNIPAVYFLTTAAASSSIGLHAFKNPGEKYPFPDFYDNSNITPEPPSADNMKLLHDFIACFERSCDIILIKSFRELEGKYIDLLSTLSDKTLVPVGPLVQDPMGHNEDPKTEQIINWLDKRAESTVVFVCFGSEYFLSNEELEEVAIGLEISTVNFIWAVRLIEGEKKGILPEGFVQRVGDRGLVVEGWAPQARILGHSSTGGFVSHCGWSSIAEIMKFGVPVIAMARHLDQPLNGKLAAEVGVGMEVVRDENGKYKREGIAEVIRKVVVEKSGEVIRRKARELSEKMKEKGEQEIDRALEELVQICKKKKDEQ

SEQ ID NO: 123 - DNA, 1329 bp, Panax ginseng
  misc_feature 1..1329: note = GT29-23
  source 1..1329: mol_type = other DNA
atggataaccaagaaggtagaatcagtatagcgttgctaccatttttagcccatggtcacatatctcccttctttgagctagccaaacaactcgcaaaaagaaattgcaatgttttcctctgttctaccccaatcaatcttagctccatcaagaataaggattcctctgcttctataaaactagttgagcttcatcttccatcttcccctgatcttcctcctcactatcacaccacaaatggcctcccttcccatctcatggtcccactcataaacgcctttgaaacagcaggccccaccttctctgaaatccttaaaaccttaaaccccgatttgcttatttatgatttcaatccctcatgggcaccggagatcgcttcgtctcacaatattccggcagtttatttcctaaccacggcagcagccagctcttccattggcctacatgctttcaaaaacccaggtgaaaaatacccatttccagatttttatgataacagtaataatacccctgaaccaccttctgcagataacatgaagctacttcatgattttatcgcttgtttcgaacgatcttgcgacattattttgattaagagttttatagaactagaagggaaatatatcgatttgctttccactttatctgataaaactttggttcctgttggtccactcgttcaagatcctatgggccataatgaagatccaaaaacagagcagattataaactggcttgacaaaagggctgaatctacagtggtgtttgtctgctttggaagtgagtattttctctccaatgaggaattggaagaagtagcaattgggctagagattagcatggttaatttcatatgggctgtgagattaattgaaggagagaaaaaaggggttttaccagagggatttgttcaaagggtaggagacagaggattggttgtggaggggtgggctccacacgcaagaattttaggacattcaagcaccggtgggtttgtgagccattgtgggtggagttctattgcggagagtatgaagtttggggttccagtaattgccatggccaggcatcttgatcagcctttgaatggtaagctggcggcggaggttggtgtgggcatggaggttgtgagagatgaaaatgggaagtataagagagaagggattgcagaggtaataagaaaagtcgttgtggagaaaagtggggaggttatgaggaggaaagcaagggaattgagtgagaaaatgaaagagaaaggagaggaagagattgatagggcagtggaggagctagtacaaatttgtaagaagaagaaagatgcacaatag

SEQ ID NO: 124 - protein, 442 aa, Panax ginseng
  REGION 1..442: note = misc_feature - GT29-23
MDNQEGRISIALLPFLAHGHISPFFELAKQLAKRNCNVFLCSTPINLSSIKNKDSSASIKLVELHLPSSPDLPPHYHTTNGLPSHLMVPLINAFETAGPTFSEILKTLNPDLLIYDFNPSWAPEIASSHNIPAVYFLTTAAASSSIGLHAFKNPGEKYPFPDFYDNSNNTPEPPSADNMKLLHDFIACFERSCDIILIKSFIELEGKYIDLLSTLSDKTLVPVGPLVQDPMGHNEDPKTEQIINWLDKRAESTVVFVCFGSEYFLSNEELEEVAIGLEISMVNFIWAVRLIEGEKKGVLPEGFVQRVGDRGLVVEGWAPHARILGHSSTGGFVSHCGWSSIAESMKFGVPVIAMARHLDQPLNGKLAAEVGVGMEVVRDENGKYKREGIAEVIRKVVVEKSGEVMRRKARELSEKMKEKGEEEIDRAVEELVQICKKKKDAQ

SEQ ID NO: 125 - DNA, 48 bp, synthetic construct; identical to SEQ ID NO: 103
  misc_feature 1..48: note = primer
  source 1..48: mol_type = other DNA

SEQ ID NO: 126 - DNA, 46 bp, synthetic construct
  misc_feature 1..46: note = primer
  source 1..46: mol_type = other DNA
tcgagtgcggccgcaagcttgtcgacttgttcatctttcttcttct

SEQ ID NO: 127 - DNA, 48 bp, synthetic construct; identical to SEQ ID NO: 101
  misc_feature 1..48: note = primer
  source 1..48: mol_type = other DNA

SEQ ID NO: 128 - DNA, 46 bp, synthetic construct; identical to SEQ ID NO: 126
  misc_feature 1..46: note = primer
  source 1..46: mol_type = other DNA

SEQ ID NO: 129 - DNA, 48 bp, synthetic construct; identical to SEQ ID NO: 101
  misc_feature 1..48: note = primer
  source 1..48: mol_type = other DNA

SEQ ID NO: 130 - DNA, 46 bp, synthetic construct; identical to SEQ ID NO: 126
  misc_feature 1..46: note = primer
  source 1..46: mol_type = other DNA

SEQ ID NO: 131 - DNA, 48 bp, synthetic construct; identical to SEQ ID NO: 101
  misc_feature 1..48: note = primer
  source 1..48: mol_type = other DNA

SEQ ID NO: 132 - DNA, 46 bp, synthetic construct; identical to SEQ ID NO: 126
  misc_feature 1..46: note = primer
  source 1..46: mol_type = other DNA

SEQ ID NO: 133 - DNA, 48 bp, synthetic construct; identical to SEQ ID NO: 103
  misc_feature 1..48: note = primer
  source 1..48: mol_type = other DNA

SEQ ID NO: 134 - DNA, 46 bp, synthetic construct
  misc_feature 1..46: note = primer
  source 1..46: mol_type = other DNA
tcgagtgcggccgcaagcttgtcgacttgtgcatctttcttcttct

(19) United States
(12) Patent Application Publication
(10) Pub. No.: US 2025/0331481 A1
(43) Pub. Date: Oct. 30, 2025

(54) GROUP OF UDP-GLYCOSYLTRANSFERASE FOR CATALYZING CARBOHYDRATE CHAIN ELONGATION, AND APPLICATION THEREOF

(71) Applicant: GSYNBIOT (SHANGHAI) CO., LTD, Shanghai (CN)

(72) Inventors: Zhihua ZHOU, Shanghai (CN); Wei WEI, Shanghai (CN); Xing YAN, Shanghai (CN); Chengshuai YANG, Shanghai (CN); Chaojing LI, Shanghai (CN); Yongjun WEI, Shanghai (CN); Pingping WANG, Shanghai (CN)

(21) Appl. No.: 19/263,621

(22) Filed: Jul. 9, 2025

Related U.S. Application Data
(62) Division of application No. 17/976,715, filed on Oct. 28, 2022, now Pat. No. 12,356,911.
Application No. 17/976,715 is a division of application No. 16/614,944, filed on Nov. 19, 2019, now Pat. No. 11,542,484.

(30) Foreign Application Priority Data
May 19, 2017 (CN) 201710359069.7

(51) Int. Cl. A01H 5/06; A01H 1/00; C12N 9/10; C12P 19/18

(52) U.S. Cl. CPC A01H 5/06; A01H 1/00; C12N 9/1048; C12P 19/18

(57) ABSTRACT

This invention relates to glycosyltransferase and an application thereof. Specifically, provided is using glycosyltransferase GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, and PNUGT29-15, and derived polypeptides thereof to catalyze the first glycosyl at position C-20, the first glycosyl at position C-6, and the first glycosyl at position C-3 of a tetracyclic triterpene compound substrate to elongate a carbohydrate chain, thereby obtaining a catalytic reaction of ginsenoside products such as ginsenoside Rg3, ginsenoside Rd, ginseno-side Rb 1, ginsenoside Rb3, saponin DMGG, saponin DMGX, gypenoside LXXV, gypenoside XVII, gypenoside XIII, gypenoside IX, notoginsenoside U, and notoginsenoside R1, notoginsenoside R2, notoginsenoside R3, 3-0-13-(D-xylopyranosyl)-13-(D-glucopyra-nosyl)-PPD, 3-0-13-(D-xylopyranosyl)-13-(D-glucopyranosyl)-CK, 20-O-Glucosylginsenoside Rf, and Ginsenoside F3. The glycosyltransferase can further be applied to construction of artificially synthesized ginsenoside, novel ginsenoside, and derivatives thereof.

### CROSS-REFERENCE TO RELATED APPLICATIONS

The present application is a divisional application of U.S. patent application Ser. No. 17/976,715, filed Oct. 28, 2022, which is a divisional application of U.S. patent application Ser. No. 16/614,944, filed Nov. 19, 2019, and issued as U.S. Pat. No. 11,542,484 on Jan. 3, 2023, which is the 35 U.S.C.371 National Stage of International Application Number PCT/CN2018/087678, filed May 21, 2018, which claims priority of Chinese Patent Application No. CN201710359069.7, filed May 19, 2017, the content of each is incorporated herein by reference in its entirey.

### INCORPORATION OF SEQUENCE LISTING

This application contains a sequence listing submitted in Computer Readable Form (CRF). The CRF file contains the sequence listing entitled “4-2-BA4080204DIV-SequenceListing.xml”, which was created on Jun. 23, 2025, and is 220,179 bytes in size. The information in the sequence listing is incorporated herein by reference in its entirety.

### TECHNICAL FIELD

The present invention relates to the field of biotechnology and plant biology, and in particular, the present invention relates to a group of glycosyltransferases and uses thereof.

### BACKGROUND

Ginsenoside is a generic term for saponins isolated from the plants of Panax genus (such as ginseng, Panax notoginseng, American ginseng, etc.) and Gynostemma pentaphyllum, and is a class of triterpenoids. Ginsenosides may also be called as ginsenosides, notoginsenosides, and gypenosides depending on the source from which they are isolated. Ginsenosides are the main biologically active ingredient in these medicinal plants. Currently, about 150 kinds of saponins have been isolated. Structurally, ginsenosides are mainly bioactive small molecules formed by glycosylation of sapogenins. There are only a few saponins of ginsenosides, mainly of which are protopanoxadiol and protopanaxatriol of dammarane type tetracyclic triterpenes, and oleanolic acid. Glycosylation of sapogenin can increase its water solubility, alter its subcellular localization, and produce different biological activities. Most of the protopanaxadiol saponins are glycosylated on the C3 and/or C20 hydroxyl groups, while the protopanaxatriol saponins are glycosylated on the C6 and/or C20 hydroxyl groups. Different types of glycosylation and varying degrees of glycosylation modification produce ginsenosides with a multitude of molecular structures.

Ginsenosides with different glycosylation modifications have different biological activities. For example, Rb1, Rb2 and Rb3 are Rds with a molecule of glucose, arabinose and xylose extended on the C20-O-Glc, respectively. The experiment has confirmed that the rich saponin Rb1 has the effects of protecting nerve cells and anti-inflammation and anti-oxidation; Rb2 has the effects of inhibiting tumor angiogenesis and tumor metastasis, reducing blood glucose in diabetic mice and reducing blood lipid; Rb3 has the effects of slowing down myocardial ischemia and anti-depression.

Ginsenosides are prepared by using total saponins of ginseng or panax notoginseng or rich saponins as raw materials, depending on a hydrolysis method of chemical, enzymatic and microbial fermentation. Since wild ginseng resources have been basically depleted, ginsenoside resources are currently derived from artificial cultivation of ginseng or notoginseng. Their artificial cultivation has a long growth cycle (generally 5-7 years or more) and is geographically restricted. It is often subject to pests and diseases, thereby requiring a large amount of pesticides. Therefore, there is a serious continuous cropping obstacle during the artificial cultivation of ginseng or Panax notoginseng (the ginseng or Panax notoginseng plantation needs to fallow for more than 5-15 years to overcome the continuous cropping obstacle), so the yield, quality and security of ginsenosides are all facing challenges.

The development of synthetic biology offers new opportunities for heterologous synthesis of plant-derived natural products. Using yeast as a chassis, through the assembly and optimization of metabolic pathways, it has been realized to synthesize artemisinic acid or dihydroartemisinic acid with cheap monosaccharides, and then to produce artemisinin by one-step chemical conversion, which indicates the synthetic biology has a great potential for drug synthesis in natural products. Ginsenoside monomers are heterologous synthesized by synthetic biological methods using the yeast chassis cells, and the raw materials are cheap monosaccharides, and the preparation process is a safe and controllable fermentation process, thereby avoiding any external contamination (for example, pesticides used in the artificial planting of raw plants). Therefore, the preparation of ginsenoside monomer by synthetic biology technology not only has cost advantages, but also ensures the quality and safety of the finished product. Synthetic biological techniques are used to prepare a sufficient amount of various high-purity natural and non-natural ginseno side monomers for activity determination and clinical experiments to promote the development of innovative drugs for rare ginsenosides.

In recent years, through the transcriptome and functional genomic studies on ginseng, notoginseng and American ginseng, the analysis of the saponin synthesis pathway of ginsenosides has made great progress. In 2006, Japanese and Korean scientists identified the terpenoid cyclase element (dammarenediol synthase, PgDDS), which converts epoxy squalene to dammarene diol. From 2011 to 2012, Korean scientists further identified cytochrome P450 elements CYP716A4 and CYP716A53v2, which oxidize dammarene diol to protopanaxadiol and further oxidize protopanaxadiol to protopanaxatriol.

The artificial synthesis of these pharmaceutically active ginsenosides by synthetic biological methods requires not only the construction of a metabolic pathway for the synthesis of sapogenins, but also the identification of a UDP-glycosyltransferase that catalyzes the glycosylation of ginsenosides. The function of UDP-glycosyltransferase is to transfer glycosyl groups from glycosyl donors (nucleoside diphosphates such as UDP-glucose, UDP-rhamnose, UDP-xylose and UDP-arabinose) to different glycosyl acceptors. According to the genome analysis of plants that have been sequenced, the plant genome often encodes hundreds of different glycosyltransferases. Since the substrates (including glycosyl donors and glycosyl acceptors) that may be catalyzed by UDP-glycosyltransferase are very diverse, the functional identification of this UDP-glycosyltransferase poses great difficulties. Until 2014, the first UDP-glycosyltransferase (UGTPg1) involved in ginsenoside glycosylation was identified by Chinese scholars, which can be transferred to a glucosyl group on the C20 hydroxyl group of the Protopanaxadiol ginsenoside. Subsequently, Korean scientists cloned two UDP-glycosyltransferase elements (PgUGT74AE2 and PgUGT94Q2) in ginseng, which can transferr a glucosyl group and a glucosyl extension to the C3 position of the Protopanaxadiol saponin. Almost at the same time, Chinese scholars also independently cloned two glycosyltransferase elements UGTPg45 and UGTPg29, which have the same functions as PgUGT74AE2 and PgUGT94Q2, from ginseng. In 2015, Chinese scholars further identified a UDP-glycosyltransferase element (UGTPg100) that can transferr a glucosyl group to the C6 position of the Protopanaxatriol. In 2015, Korean scholars discovered a glycosyltransferase GpUGT23 that extends a glucosyl group on C20 of Protopanaxadiol and protopanaxatriol saponin in Gynostemma pentaphyllum. However, up to now, in addition to a glycosyltransferase plant extending a glycosyl at the C3 position, other glycosyltransferases in ginseng that catalyze the extension of the carbohydrate chain have not been reported.

Under this background, the inventors have cloned and identified the glycosyltransferase which can extend a glucosyl or xylosyltaxol on the C20 of the Protopanaxadiol and protopanaxatriol saponin and the glycosyltransferase which can extend a xylosyltaxol on the C6 of the protopanaxatriol saponin. The glycosyltransferase can be used for the preparation of ginsenosides including ginsenoside Rb1, ginsenoside Rb3, gypenoside LXXV, gypenoside XVII, notoginsenoside U, notoginsenoside R1, notoginsenoside R2 and notoginsenoside R3.

### SUMMARY OF THE INVENTION

The present invention provides a novel set of glycosyltransferases and a method for catalyzing a glycosylation reaction of a tetracyclic triterpenoid using the glycosyltransferases.

In a first aspect of the present invention, it provides an in vitro glycosylation method, comprising the steps of:
transferring a glycosyl group from the glycosyl donor to the following positions of the tetracyclic triterpenoid in the presence of a glycosyltransferase:
the first glycosyl group on position C20 and/or position C3;
thereby forming a glycosylated tetracyclic triterpenoid;
wherein the glycosyltransferase is selected from the group consisting of:
a glycosyltransferase as shown in SEQ ID NO: 4, 6, 8, 8, 39, 41, 43, 45, 47, 49, 51, 53, 55, 57, 59, 98, or 100 or a derivative polypeptide thereof.

In another preferred embodiment, the tetracyclic triterpenoids glycosylated at the position C20 include ginsenosides Rd, CK, F1 and F2.

In a second aspect of the present invention, it provides an in vitro glycosylation method, comprising the steps of:
transferring a glycosyl group from a glycosyl donor to the following positions of the tetracyclic triterpenoid in the presence of a glycosyltransferase:
the first glycosyl group on position C6;
thereby forming a glycosylated tetracyclic triterpenoid;
wherein the glycosyltransferase is selected from the group consisting of:
a glycosyltransferase as shown in SEQ ID NO: 12, 14, 16, 18, 20, 22, 24, 26, 28 and 30 or a derivative polypeptide thereof.

In another preferred embodiment, the tetracyclic triterpenoids glycosylated at the position C6 includes Rg1 or Rh1.

The present invention provides a method for in vitro glycosylation comprising the steps of:
transferring a glycosyl group from a glycosyl donor to the following positions of the tetracyclic triterpenoid in the presence of a glycosyltransferase:
the first glycosyl group on position C3;
thereby forming a glycosylated tetracyclic triterpenoid;
wherein the glycosyltransferase is selected from the group consisting of:
a glycosyltransferase as shown in SEQ ID NO: 39, 41, 43, 45, 47, 49, 51, 53, 55, 57, 59, 90, 92, 94, 96, 98, 100, 116, 118, 120, 122, and 124 or a derivative polypeptide thereof.

In another preferred embodiment, the tetracyclic triterpenoids glycosylated on the position C3 includes F2 or Rh2.

In another preferred embodiment, the derivative polypeptide is independently selected from the group consisting of:
(a) a polypeptide of any one or more of the amino acid sequences as shown in SEQ ID NOs.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, and 124;
(b) a derivative polypeptide formed by adding a tag sequence, a signal sequence or a secretion signal sequence to SEQ ID NOs: 4, 6, 8, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124 and having glycosyltransferase activity;
(c) a derivative polypeptide of an amino acid sequence having an identity of 95% with the amino acid sequence of any one or more of SEQ ID NOs: 4, 6, 8, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, and 124 and having glycosyltransferase activity.

In another preferred embodiment, (c) further includes a derivative polypeptide formed by substitution, deletion or addition of one or several amino acid residues of any one or more of the amino acid sequences of SEQ ID NOs.: 4, 6, 8, 14, 16, 18, 20, 22, 24, 26, 28, 30, 39, 41, 43, 45 47, 49, 51, 53, 55, 57, 59, 90, 92, 94, 96, 98, 100, 116, 118, 120, 122, and 124 and having the glycosyltransferase activity.

In a third aspect of the present invention, it provides an isolated polypeptide, wherein the isolated polypeptide is:
a polypeptide or a derivative polypeptide thereof of any one or more of the amino acid sequences as shown in SEQ ID NOs.: 4, 6, 8, 28, 30, 39, 41, 43, 45, 47, 49, 51, 53, 55, 57, 59, 90, 92, 94, 96, 98, 100, 116, 118, 120, 122, and 124; wherein the derivative polypeptide is selected from the group consisting of:
(a) a polypeptide of any one or more of the amino acid sequences as shown in SEQ ID NOs.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, and 124;
(b) a derivative polypeptide formed by adding a tag sequence, a signal sequence or a secretion signal sequence to SEQ ID NOs: 4, 6, 8, 28, 30, 39, 41, 43, 45, 47, 49, 51, 53, 55, 57, 59, 90, 92, 94, 96, 98, 100, 116, 118, 120, 122, or 124 and having a glycosyltransferase activity;
(c) a derivative polypeptide of an amino acid sequence having an identity of _95% with the amino acid sequence as shown in any one or more of SEQ ID NOs: 4, 6, 8, 28, 30, 39, 41, 43, 45, 47, 49, 51, 53, 55, 57, 59, 90, 92, 94, 96, 98, 100, 116, 118, 120, 122, and 124 and having a glycosyltransferase activity.

In another preferred embodiment, (c) further includes a derivative polypeptide formed by substitution, deletion or addition of one or several amino acid residues of any one or more of the amino acid sequences of SEQ ID NOs.: 4, 6, 8, 28, 30, 39, 41, 43, 45, 47, 49, 51, 53, 55, 57, 59, 90, 92, 94, 96, 98, 100, 116, 118, 120, 122, and 124 and having a glycosyltransferase activity.

In another preferred embodiment, the isolated polypeptide is used for in vitro glycosylation.

In a fourth aspect of the present invention, it provides an isolated polynucleotide, wherein the polynucleotide is selected from the group consisting of:
(A) a nucleotide sequence encoding a polypeptide as shown in SEQ ID NOs: 4, 6, 8, 28, 30, 39, 41, 43, 45, 47, 49, 51, 53, 55, 57, 59, 90, 92, 94, 96, 100, 116, 118, 120, 122 or 124;
(B) a nucleotide sequence as shown in SEQ ID NO.: 3, 5, 7, 27, 29, 38, 40, 42, 44, 46, 48, 50, 52, 54, 56, 58, 89, 91, 93, 95, 97, 99, 115, 117, 119, 121, or 123;
(C) a nucleotide sequence having an identity of _95% (preferably _98%) with a nucleotide sequence as shown in SEQ ID NO.: 3, 5, 7, 27, 29, 38, 40, 42, 44, 46, 48, 50, 52, 54, 56, 58, 89, 91, 93, 95, 97, 99, 115, 117, 119, 121, or 123;
(D) a nucleotide sequence complementary (preferably completely complementary) to the nucleotide sequence of any of (A)-(C).

In another preferred embodiment, (D) further includes a nucleotide sequence formed by truncation or addition of 1-60 (preferably 1-30, more preferably 1-10) nucleotides at 5′ end and/or 3′ end of the nucleotide sequences of SEQ ID NOs.: 3, 5, 7, 27, 29, 38, 40, 42, 44, 46, 48, 50, 52, 54, 56, 58, 89, 91, 93, 95, 97, 99, 115, 117, 119, 121, or 123.

In another preferred embodiment, the nucleotide sequences as shown in SEQ ID NO: 3, 5, 7, 27, 29, 38, 40, 42, 44, 46, 48, 50, 52, 54, 56, 58, 89, 91, 93, 95, 97, 99, 115, 117, 119, 121, or 123, encoding the polypeptides as shown in SEQ ID NOs: 4, 6, 8, 28, 30, 39, 41, 43, 45, 47, 49, 51, 53, 55, 57, 59, 90, 92, 94, 96, 98, 100, 116, 118, 120, 122, or 124.

In a fifth aspect of the present invention, it provides a vector comprising the polynucleotide according to the fourth aspect of the present invention, or expressing the isolated polypeptide according to the third aspect of the present invention.

Use of the isolated polypeptide according to the third aspect of the present invention for catalyzing one or more of the following reactions, or preparing a catalytic formulation which catalyzes one or more of the following reactions:
transferring the glycosyl group from the glycosyl donor to the following positions of the tetracyclic triterpenoid to extend the carbohydrate chain:
(i) the first glycosyl group on position C-6;
(ii) the first glycosyl group on position C-20; and/or
(iii) the first glycosyl group on position C3.

In another preferred embodiment, the glycosyl group transfer comprises the addition or substitution of a glycosyl group on a specific position.

In another preferred embodiment, it also provides a use of a polypeptide or a derivative polypeptide thereof for catalyzing the following reactions or for preparing a catalytic formulation which catalyzes the following reactions:
transferring a glycosyl group from a glycosyl donor to the following positions of the tetracyclic triterpenoid in the presence of a glycosyltransferase:
the first glycosyl group on position C-6; or the first glycosyl group on position C-20; and/or the first glycosyl group on position C-3;
thereby forming a glycosylated tetracyclic triterpenoid;
wherein the glycosyltransferase is selected from the group consisting of:
a glycosyltransferase, or a derivative polypeptide thereof as shown in SEQ ID NO: 4, 6, 8, 12, 14, 16, 18, 20, 22, 24, 26, 28, 30, 39, 41, 43, 45, 47, 49, 51, 53, 55, 57, 59, 90, 92, 94,96, 98, 100, 116, 118, 120, 122, and 124.

In another preferred embodiment, it also provides a use of a polypeptide or a derivative
polypeptide thereof for catalyzing the following reactions, or for preparing a catalytic formulation which catalyzes the following reactions:
transferring a glycosyl group from glycosyl donor to the following positions of the tetracyclic triterpenoid in the presence of a glycosyltransferase:
the first glycosyl group on position C20 and/or position C3;
thereby forming a glycosylated tetracyclic triterpenoid;
wherein the glycosyltransferase is selected from the group consisting of:
a glycosyltransferase or a derivative polypeptide thereof as shown in SEQ ID NO: 4, 6, 8, 39, 41, 43, 45, 47, 49, 51, 53, 55, 57, 59, 98, and 100.

In another preferred embodiment, it also provides a use of a polypeptide or a derivative polypeptide thereof for catalyzing the following reactions or for preparing a catalytic formulation which catalyzes the following reactions:
transferring a glycosyl group from a glycosyl donor to the following positions of the tetracyclic triterpenoid in the presence of a glycosyltransferase:
the first glycosyl group on position C6;
thereby forming a glycosylated tetracyclic triterpenoid;
wherein the glycosyltransferase is selected from the group consisting of:
a glycosyltransferase or a derivative polypeptide thereof as shown in SEQ ID NO: 12, 14, 16, 18, 20, 22, 24, 26, 28, and 30.

In another preferred embodiment, it also provides a use of a polypeptide or a derivative polypeptide thereof for catalyzing the following reactions or for preparing a catalytic formulation which catalyzes the following reactions:
transferring a glycosyl group from a glycosyl donor to the following positions of the tetracyclic triterpenoid in the presence of a glycosyltransferase:
the first glycosyl group on position C-3;
thereby forming a glycosylated tetracyclic triterpenoid;
wherein the glycosyltransferase is selected from the group consisting of:
a glycosyltransferase or a derivative polypeptide thereof as shown in SEQ ID NO: 39, 41, 43, 45, 47, 49, 51, 53, 55, 57, 59, 90, 92, 94, 96, 98, 100, 116, 118, 120, 122, and 124.

In another preferred embodiment, the derivative polypeptide is each selected from the group consisting of:
(a) a polypeptide having an amino acid sequence of any one of SEQ ID NOs.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, and 124;
(b) a derivative polypeptide formed by adding a tag sequence, a signal sequence or a secretion signal sequence to SEQ ID NOs: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124 and having a glycosyltransferase activity;
(c) a derivative polypeptide of an amino acid sequence having an identity of 95% with the amino acid sequence as shown in any one of SEQ ID NOs: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, and 124 and having a glycosyltransferase activity.

In another preferred embodiment, the glycosyl donor comprises a nucleoside diphosphate selected from the group consisting of UDP-glucose, ADP-glucose, TDP-glucose, CDP-glucose, GDP-glucose, UDP-acetylglucose, ADP-acetylglucose, TDP-acetylglucose, CDP-acetylglucose, GDP-acetylglucose, UDP-xylose, ADP-xylose, TDP-xylose, CDP-xylose, GDP-xylose, UDP-galacturonic acid, ADP-galacturonic acid, TDP-galacturonic acid, CDP-galacturonic acid, GDP-galacturonic acid, UDP-galactose, ADP-galactose, TDP-galactose, CDP-galactose, GDP-galactose, UDP-arabinose, ADP-arabinose, TDP-arabinose, CDP-arabinose, GDP-arabinose, UDP-rhamnose, ADP-rhamnose, TDP-rhamnose, CDP-rhamnose, GDP-rhamnose, UDP-xylose, ADP-xylose, TDP-xylose, CDP-xylose, GDP-xylose, and other nucleoside diphosphate hexose and nucleoside pentose diphosphate, and a combination thereof.

In another preferred embodiment, the glycosyl donor comprises a uridine diphosphate (UDP) saccharide selected from the group consisting of UDP-glucose, UDP-galacturonic acid, UDP-galactose, UDP-arabinose, UDP-rhamnose, UDP-xylose, and other uridine diphosphate hexose and uridine pentose diphosphate, and a combination thereof.

In another preferred embodiment, the isolated polypeptide is used to catalyze one or more of the following reactions or to prepare a catalytic formulation which catalyzes one or more of the following reactions:

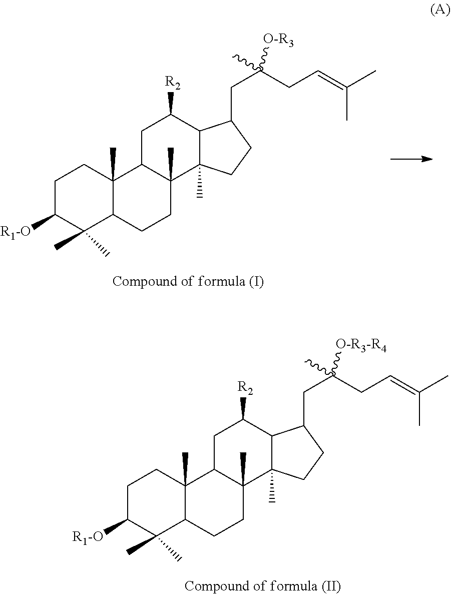

wherein, R1 is H, a monosaccharide glycosyl or a polysaccharide glycosyl; R2 is H or OH; R3 is a monosaccharide glycosyl; and R4 is a monosaccharide glycosyl; and the polypeptide is selected from the group consisting of SEQ ID NOs: 4, 6, 8, 39, 41, 43, 45, 47, 49, 51, 53, 55, 57, 59, 98, 100, 116, 118, 120, 122, or 124 or a derivative polypeptide thereof.

In another preferred embodiment, the monosaccharide comprises glucose (Glc), rhamnose (Rha), acetylglucose (G1c (6) Ac), arabinofuranose (Araf), arabian pyranose (Arap), or xylose (Xyl), etc.

In another preferred embodiment, the polysaccharide comprises a polysaccharide consisting of 2-4 monosaccharides such as Glc(2-1)Glc, Glc(6-1)Glc, Glc(6)Ac, Glc(2-1)Rha, Glc(6-1)Arap, Glc(6-1)Xyl, Glc(6-1)Araf, Glc(3-1)Glc(3-1), Glc(2-1)Glu (6)Ac, Glc(6-1)Arap (4-1)Xyl, Glc(6-1)Arap(2-1)Xyl, or Glc(6-1)Arap (3-1)Xyl.

In another preferred embodiment, the compound with the substitution of R1-R4 is shown in the following table:

| substrate | R1 | R2 | R3 | R4 | product |
|---|---|---|---|---|---|
| CK | H | OH | Glc | Glc | Gypenoside |
| | | | | | LXXV |
| DMG | H | H | Glc | Glc | DMGG |
| F2 | Glc | OH | Glc | Glc | Gypenoside |
| | | | | | XVII |
| Rd | Glc(2-1)Glc | OH | Glc | Glc | Rbl |
| CK | H | OH | Glc | Xyl | Gypenoside |
| | | | | | XIII |
| DMG | H | H | Glc | Xyl | DMGX |
| F2 | Glc | OH | Glc | Xyl | Gypenoside |
| | | | | | IX |
| Rd | Glc(2-1)Glc | OH | Glc | Xyl | Rb3 |
| CK | H | OH | Glc | Arabinose | Ginsenoside |
| | | | | | F3 |

that is, when R1 is H, R2 is OH, and R3 is a glucosyl, the compound of formula (I) is ginsenoside CK (CK);
when the R1 is H, R2 is OH, and both R3 and R4 are glucosyls, the compound of the formula (II) is Gypenoside LXXV;
when the R1 is H, R2 is OH, R3 is a glucosyl, and R4 is a xylose group, the compound of the formula (II) is Gypenoside XIII;
when both R1 and R2 are H and R3 is a glucosyl, the compound of formula (I) is ginsenoside DMG;
when both R1 and R2 are H, and both R3 and R4 are glucosyls, the compound of formula (II) is saponin DMGG (20-O-β-(D-glucopyranosyl)-β-(D-glucopyranosyl)-dammarenediol);
when both R1 and R2 are H, R3 is a glucosyl, and R4 is a xylose group, the compound of formula (II) is saponin DMGX (20-O-β-(D-xylopyranosyl)-β-(D-glucopyranosyl)-dammarenediol);
when R1 is a glucosyl, R2 is OH, and R3 is a glucosyl, the compound of formula (I) is ginsenoside F2 (F2);
when R1 is a glucosyl, R2 is OH, and both R3 and R4 are glucosyls, the compound of formula (II) is Gypenoside XVII;
when R1 is a glucosyl, R2 is OH, R3 is a glucosyl, and R4 is a xylose group, the compound of formula (II) is Gypenoside IX;
when R1 is two glucosyls (Glc(2-1) Glc), R2 is OH, and R3 is a glucosyl, the compound of formula (I) is ginsenoside Rd;
when R1 is two glucosyls (Glc(2-1) G1c), R2 is OH, and both R3 and R4 are glucosyls, the compound of formula (II) is ginsenoside Rb1; or
when R1 is two glucosyls (Glc(2-1) Glc), R2 is OH, R3 is a glucosyl, and R4 is a xylose group, the compound of formula (II) is ginsenoside Rb3;
when the R1 is H, R2 is OH, R3 is a glucosyl, and R4 is an arabinose group, the compound of the formula (II) is ginsenoside F3;

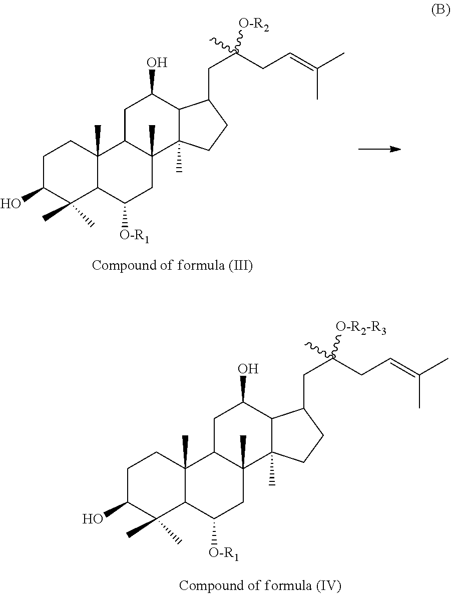

Wherein, R1 is H, a glycosyl or polysaccharide glycosyl group, R2 is a glycosyl group, and R3 is a glycosyl group, and the polypeptide is selected from the group consisting of SEQ ID NOs.: 4 and a derivative polypeptide thereof.

In another preferred embodiment, the compound with the substitution of R1-R3 is shown in the following table:

| substrate | R1 | R2 | R3 | product |
|---|---|---|---|---|
| Fl | H | Glc | Glc | notoginsenoside U |
| Rg 1 | Glc | Glc | Glc | notoginsenoside R3 |

that is, when R1 is H, and R2 is a glucosyl, the compound of the formula (III) is ginsenoside F1(F1);
when R1 is H, and both R2 and R3 are glucosyls, the compound of the formula (IV) is notoginsenoside U; when R1 and R2 are glucosyls, the compound of the formula (III) is ginsenoside Rg1 (Rg1); or
when R1, R2 and R3 are glucosyl groups, the compound of the formula (IV) is notoginsenoside R3 (R3);

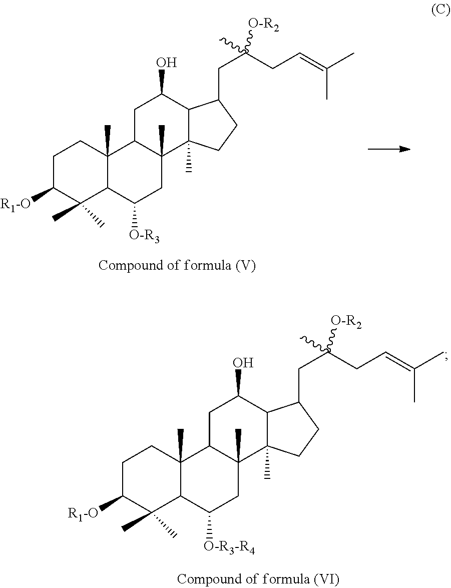

wherein, R1 and R2 are H or glycosyls, and R3 and R4 are glycosyls; and the polypeptide is selected from the group consisting of SEQ ID NOs.: 12, 14, 16, 18, 20, 22, 24, 26, 28, 30 or a derivative polypeptide.

In another preferred embodiment, the compound with the substitution of R1-R4 is shown in the following table:

| substrate | R1 | R2 | R3 | R4 | product |
|---|---|---|---|---|---|
| Rg1 | H | Glc | Glc | Xyl | notoginsenoside R1 |
| Rg1 | H | Glc | Glc | Glc | 20-O-Glucosylginsenoside Rf |
| Rhl | H | H | Glc | Xyl | notoginsenoside R2 |
| Rhl | H | H | Glc | Glc | ginsenoside Rf |

that is, when R1 is H, and both R2 and R3 are glucosyls, the compound of the formula is ginsenoside Rg1;
when R1 is H, R2 and R3 are glucosyls, and R4 is a xylose group, the compound of formula (VI) is notoginsenoside R1;
when R1 is H, R2 and R3 are glucosyls, and R4 is a glucosyl, the compound of formula (VI) is saponin 20-O-Glucosylginsenoside Rf;
when R1 and R2 are H, and R3 is a glucosyl, the compound of formula (V) is ginsenoside Rh1;
when R1 and R2 are H, R3 is a glucosyl, and R4 is a xylose group, the compound of the formula (VI) is notoginsenoside R2; when R1 and R2 are H, R3 and R4 are glucosyls, the compound of formula (VI) is ginsenoside Rf.

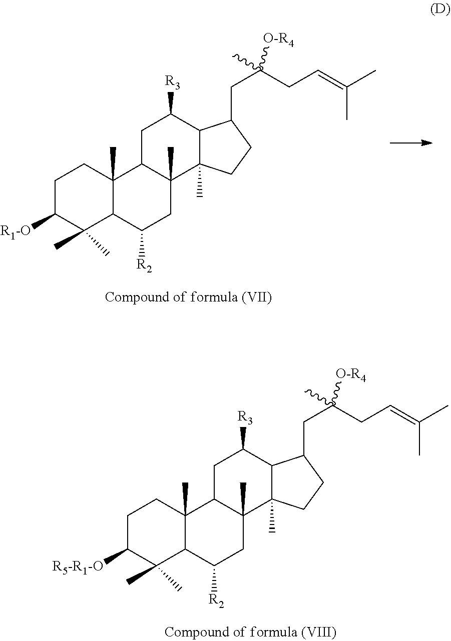

wherein, R1 is a glycosyl; R2 and R3 are OH or H; R4 is a glycosyl or H; R5 is a glycosyl, and R5-R1-O is a first glycosyl-derived glycosyl at C3 position; and the polypeptide is selected from the group consisting of SEQ ID NOs.: 39, 41, 43, 45, 47, 49, 51, 53, 55, 57, 59, 98, 100, 116, 118, 120, 122, and 124 and a derivative polypeptide thereof;
the compound with the substitution of R1-R4 is shown in the following table:

| substrate | R1 | R2 | R3 | R4 | R5 | product |
|---|---|---|---|---|---|---|
| Rh2 | Glc | H | OH | H | Glc | Rg3 |
| F2 | Glc | H | OH | Glc | Glc | Rd |
| Gypenoside | Glc | H | OH | Glc(6,1)Glc | Glc | Rb1 |
| XVII | | | | | | |
| Gypenoside | Glc | H | OH | Glc(6,1)xyl | Glc | Rb3 |
| IX | | | | | | |

that is, when RI is a glucosyl; R2 is H, R3 is OH, R4 is H, and the compound of the formula (VII) is Rh2;
R1 is a glucosyl; R2 is H, R3 is OH, R4 is a glucosyl, and the compound of the formula (VII) is F2;
R1 is a glucosyl; R2 is H, R3 is OH, R4 is two glucosyls, and the compound of formula (VII) is Gypenoside XVII;
R1 is a glucosyl; R2 is H, R3 is OH, R4 is a glucosyl group with a xylosyl group extended, and the compound of formula (VII) is Gypenoside IX;
when the substrate of (VII) compound is Rh2, the product of formula (VIII) is Rg3; when the substrate of (VII) compound is F2, the product of formula (VIII) is Rd; when the substrate of (VII) compound is Gypenoside XVII, the product of formula (VIII) is Rbl; when the substrate of (VII) compound is Gypenoside IX, the product of formula (VIII) is Rb3.

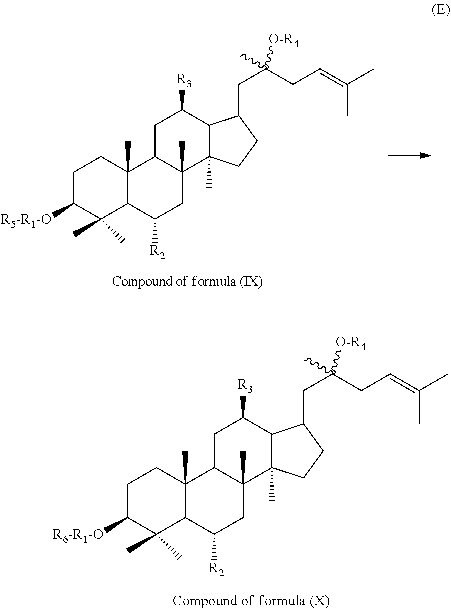

wherein, R1 is a glycosyl; R2 and R3 are OH or H; R4 is a glycosyl or H; R5 is a glycosyl, R5-R1-O is a derivative glycosyl from the first glycosyl on C3 position; and R6 is a glycosyl, R6-R1-O is a derivative glycosyl from the first glycosyl on C3 position, and the polypeptide is selected from the group consisting of SEQ ID NOs.: 41, 45, 90, 92, 94 and 96 and a derivative polypeptide thereof;
R1 is two glucosyls, R2 is H, R3 is OH, R4 is H, and the compound of formula (IX) is Rg3.
R1 is two glucosyls, R2 is H, R3 is OH, R4 is a glucosyl, and the compound of formula (IX) is Rd.

In another preferred embodiment, the glycosyl is selected from the group consisting of: a glucosyl, a xylose group, a galacturonic acid group, a galactosyl, an arabinose group, a rhamnosyl, and other hexose and pentose groups.

In another preferred embodiment, the compounds of (I), (III), (V), (VII), (IX) in the reaction formula include, but are not limited to, dammarane tetracyclic triterpenoids in S or R configuration, lanostanes tetracyclic triterpenoids, apotirucallane tetracyclic triterpenoids, tirucallanes tetracyclic triterpenoids, cycloartanes (cycloaltine) tetracyclic triterpenoids, cucurbitane tetracyclic triterpenoids or meliacanes tetracyclic triterpenoid.

In another preferred embodiment, the compounds of (II), (IV), (VI), (VIII), or (X) in the reaction formula include ginsenoside Rg3, ginsenoside Rd, ginsenoside Rbl, ginsenoside Rb3, saponin DMGG, saponin DMGX, gypenoside LXXV, gypenoside XVII, gypenoside XIII, gypenoside IX, notoginsenoside U and, notoginsenoside R1, and notoginsenoside R2, notoginsenoside R3, 3-O-β-(D-xylopyranosyl)-β-(D-glucopyranos y1)-PPD; 3-O-β-(D-xylopyranosyl)-β-(D-glucopyranosyl)-CK, 20-O-Glucosylginsenoside Rf and Ginsenoside F3.

In a sixth aspect of the present invention, it provides a method for performing a glycosyl transfer catalytic reaction, comprising the steps of: performing a glycosyl transfer catalytic reaction in the presence of the polypeptide or a derivative polypeptide thereof according to the third aspect of the present invention.

In another preferred embodiment, the method further includes the steps:
in the presence of a glycosyl donor and a polypeptide and a derivative polypeptide thereof according to the third aspect of the present invention, converting the compound of formula (I) to the compound of formula (II), or converting the compound of formula (III) to the compound of formula (IV), or converting the compound of formula (V) to the compound of formula (VI); or converting the compound of formula (VII) to the compound of formula (VIII), or converting the compound (IX) to the compound of the formula (IX).

In another preferred embodiment, the method further comprises adding the polypeptide and the derivative polypeptide thereof to a catalytic reaction, respectively; and/or
adding the polypeptide and the derivative polypeptide thereof simultaneously to a catalytic reaction.

In another preferred embodiment, the method further includes co-expressing a nucleotide sequence encoding a glycosyltransferase with a key gene in the anabolic pathway of dammarenediol and/or protopanoxadiol and/or protopanaxatriol in the host cell, thereby obtaining the compound of formula (II), (IV), (VI), (VIII), or (X).

In another preferred embodiment, the host cell is a yeast cell or an E. coli cell.

In another preferred embodiment, the polypeptide is a polypeptide having an amino acid sequence as shown in SEQ ID NOs.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124 or a derivative polypeptide thereof.

In another preferred embodiment, the nucleotide sequence encoding the polypeptide is as shown in SEQ ID NOs.: 3, 5, 7, 13, 15, 17, 19, 21, 23, 25, 27, 29, 38, 40, 42, 44, 46, 48, 50, 52, 54, 56, 58, 89, 91, 93, 95, 97, 99, 115, 117, 119, 121, or 123.

In another preferred embodiment, the method further includes: providing an additive for regulating the enzyme activity to the reaction system.

In another preferred embodiment, the additive for regulating enzyme activity is: an additive that increases enzyme activity or inhibits enzyme activity.

In another preferred embodiment, the additive for regulating the enzyme activity is selected from the group consisting of Ca^{t}+, Co²+, Mn²+, Ba²+, Al³±, Ni²+, Zn²+, and Fe²+.

In another preferred embodiment, the additive for regulating the enzyme activity is a substance capable of generating Ca²+, Co²+, Mn²+, Ba²+, Al³±, Ni²+, Zn²+, or Fe²⁺ . . . . In another preferred embodiment, the glycosyl donor is a nucleoside diphosphate saccharide selected from the group consisting of UDP-glucose, ADP-glucose, TDP-glucose, CDP-glucose, GDP-glucose, UDP-xylose, ADP-xylose, TDP-xylose, CDP-xylose, GDP-xylose, UDP-galacturonic acid, UDP-acetylglucose, ADP-acetylglucose, TDP-acetylglucose, CDP-acetylglucose, GDP-acetylglucose, ADP-galacturonic acid, TDP-galacturonic acid, CDP-galacturonic acid, GDP-galacturonic acid, UDP-galactose, ADP-galactose, TDP-galactose, CDP-galactose, GDP-galactose, UDP-arabinose, ADP-arabinose, TDP-arabinose, CDP-arabinose, GDP-arabinose, UDP-rhamnose, ADP-rhamnose, TDP-rhamnose, CDP-rhamnose, GDP-rhamnose, and other nucleoside diphosphate hexose and nucleoside diphosphate pentose, and a combination thereof. In another preferred embodiment, the glycosyl donor is a uridine diphosphate saccharide, selected from the group consisting of UDP-glucose, UDP-xylose, UDP-galacturonic acid, UDP-galactose, UDP-arabinose, UDP-rhamnose, and other uridine diphosphate hexose and uridine diphosphate pentose, and a combination thereof.

In another preferred embodiment, the pH of the reaction system is: pH 4.0-10.0, preferably pH 5.5-9.0.

In another preferred embodiment, the temperature of the reaction system is: 10° C.-105° C., preferably 20° C.-50° C.

In another preferred embodiment, the key genes in the dammarenediol anabolic pathway include (but are not limited to): a dammarenediol synthase gene.

In another preferred embodiment, the key genes in the ginsenoside CK anabolic pathway include (but are not limited to): a dammarenediol synthase gene, cytochrome P450 CYP716A47 gene, gene of reductase for P450 CYP716A47, and glycosyltransferase UGTPg1 at C20 position of tetracyclic triterpenes (Genbank accession number KF377585.1), and a combination thereof. In another preferred embodiment, the key genes in the ginsenoside F1 anabolic pathway include (but are not limited to): a dammarenediol synthase gene, a cytochrome P450 CYP716A47 gene, a gene of reductase for P450 CYP716A47, a cytochrome P450

CYP716A53V2 gene and a gene of a reductase thereof and a glycosyltransferase UGTPg1 at C20 position of tetracyclic triterpene, and a combination thereof.

In another preferred example, the key genes in the ginsenoside Rg1 anabolic pathway include (but are not limited to): a dammarenediol synthase gene, a cytochrome P450 CYP716A47 gene, a gene of reductase for P450 CYP716A47, and glycosyltransferase UGTPg1 and UGTPg100 at C20 and C6 position of tetracyclic triterpenes (Genbank accession number AKQ76388.1), and a combination thereof.

In another preferred embodiment, the substrate of the glycosyl-catalyzed reaction is a compound of formula (I), (III), (V), (VII), (IX), and the products are compounds (II), (IV), (VI), (VIII), (X);

In another preferred embodiment, the compound of formula (I) is ginsenoside CK, and the compound of formula (II) is gypenosides LXXV (20-O-β-(D-glucopyranosyl)-(β-(D-glucopyranosyl)-protopanaxadiol);
or, the compound of formula (I) is ginsenoside DMG, and the compound of formula (II) is a new ginsenoside DMGG (20-O-β-(D-glucopyranosyl)-(B-(D-glucopyranosyl)-dammarenediol);
the compound of formula (I) is ginsenoside F2, and the compound of formula (II) is gypenosides XVII (3-O-β-(D-glucopyranosyl)-20-O-β-(D-glucopyranosyl)-(β-(D-glucopyranosyl)-protopanaxadiol);
or, the compound of formula (I) is ginsenoside Rd, and the compound of formula (II) is ginsenoside Rb1 (3-O-β-(D-glucopyranosyl)-β-(D-glucopyranosyl)-20-O-β-(D-glucopyranosyl)-β-(D-glucopyranosyl)-protopanaxadiol);

or, the compound of formula (I) is ginsenoside Rd, and the compound of formula (II) is ginsenoside Rb3 (3-O-β-(D-glucopyranosyl)-β-(D-glucopyranosyl)-20-O-β-(D-xylopyranosyl)-β-(D-glucopyranosyl)-protopanaxadiol);
the compound of formula (I) is ginsenoside CK, and the compound of formula (II) is gypenosides XIII;
the compound of formula (I) is ginsenoside DMG, and the compound of formula (II) is ginsenoside DMGX (20-O-β-(D-glucopyranosyl)-β-(D-glucopyranosyl)-dammarenediol);
the compound of formula (I) is ginsenoside F2, and the compound of formula (II) is gypenosides IX;
the compound of formula (I) is ginsenoside CK, and the compound of formula (II) is ginsenoside F3; in another preferred embodiment, the compound of formula (III) is ginsenoside Fp1, and the compound of formula (IV) is notoginsenoside U (20-O-β-(D-glucopyranosyl)-β-(D-glucopyranosyl)-protopanaxatriol);

In another preferred embodiment, the compound of formula (III) is ginsenoside Rg1, and the compound of formula (IV) is notoginsenoside R3;

In another preferred embodiment, the compound of formula (V) is ginsenoside Rg1, and the compound of formula (VI) is notoginsenoside R1 (6-O-β-(D-xylopyranosyl)-β-(D-glucopyranosyl)-20-O-β-(D-glucopyranosyl)-protopanaxatriol);
the compound of formula (V) is ginsenoside Rg1, and the compound of formula (VI) is 20-O-Glucosylginsenoside Rf;
the compound of formula (V) is ginsenoside Rhl, and the compound of formula (VI) is notoginsenoside R2 (6-0-β-(D-xylopyranosyl)-β-(D-glucopyranosyl)-protopanaxatriol).

Or, the compound of formula (V) is ginsenoside Rh1, and the compound of formula (VI) is ginsenoside Rf.

In another preferred embodiment, the compound of formula (III) is ginsenoside Rg1, and the compound of formula (IV) is notoginsenoside R3;

In another preferred embodiment, the compound of formula (VII) is Rh2, and the product of compound of formula (VIII) is Rg3;
the compound of formula (VII) is F2, and the product of compound of formula (VIII) is Rd;
the compound of formula (VII) is Gypenoside XVII, and the product of compound of formula (VIII) is Rb1;
the compound of formula (VII) is Gypenoside IX, and the product of compound of formula (VIII) is Rb3.

In another preferred embodiment, the compound of formula (IX) is Rg3, and the product of compound of formula (X) is 3-O-β-(D-xylopyranosyl)-β-(D-glucopyranosyl)-PPD;
the compound of formula (IX) is Rd, and the product of compound of formula (X) is 3 -O-β-(D-xylopyranosyl)-β-(D-glucopyranosyl)-CK.

In a seventh aspect of the present invention, it provides a genetically engineered host cell containing the vector according to the fifth aspect of the present invention, or with a polynucleotide according to the fourth aspect of the present invention integrated into the gene thereof.

In another preferred embodiment, the cell is a prokaryotic cell or a eukaryotic cell.

In another preferred embodiment, the host cell is a eukaryotic cell, such as a yeast cell or a plant cell.

In another preferred embodiment, the host cell is a saccharomyces cerevisiae cell.

In another preferred embodiment, the host cell is a prokaryotic cell, such as E. coli.

In another preferred embodiment, the host cell is a ginseng cell.

In another preferred embodiment, the host cell is not a cell that naturally produces compounds of formula (II), (IV), (VI), (VII), or (X).

In another preferred embodiment, the host cell is not a cell that naturally produces ginsenoside Rg3, ginsenoside Rd, ginsenoside Rb1, ginsenoside Rb3, saponin DMGG, saponin DMGX, gypenosides LXXV, gypenosides XVII, gypenosides XIII, gypenosides IX, notoginsenoside U and notoginsenoside R1, notoginsenoside R2, notoginsenoside R3, 3-O-β-(D-xylopyranosyl)-β-(D-glucopyranosyl)-PPD; 3-O-β-(D-xylopyranosyl)-β-(D-glucopyranosyl)-CK, 20-O-Glucosylginsenoside Rf or Ginsenoside F3.

In another preferred embodiment, the key genes in the dammarene glycol anabolic pathway include (but are not limited to): a dammarene glycol synthase gene.

In another preferred example, the host cell contains key genes in the ginsenoside CK anabolic pathway including (but not limited to): a dammarene glycol synthase gene, a cytochrome P450 CYP716A47 gene, and a P450 CYP716A47 reductase gene and a glycosyltransferase UGTPg1 at the C20 site of the tetracyclic triterpene, or a combination thereof.

In another preferred example, the host cell contains key genes in the ginsenoside F1 anabolic pathway including (but not limited to): a dammarene glycol synthase gene, a cytochrome P450 CYP716A47 gene, and a reductase gene for P450 CYP716A47, a cytochrome P450 CYP716A53V2 gene and a glycosyltransferase UGTPg1 on the C20 site of the tetracyclic triterpene, or a combination thereof.

In another preferred example, the key genes in the ginsenoside Rg1 anabolic pathway include (but are not limited to): a dammarene glycol synthase gene, a cytochrome P450 CYP716A47 gene, a gene of a reductase for P450 CYP716A47, and glycosyltransferase UGTPg1 and UGTPg100 (Genbank accession number AKQ76388.1) on C20 and C6 of cyclotriterpenes, or a combination thereof.

In an eighth aspect of the present invention, it provides use of the host cell according to the seventh aspect of the present invention for preparing an enzyme catalytic reagent, or for producing a glycosyltransferase, or as a catalytic cell, or for producing formula (II), (IV), (VI), (VIII) or (X) compounds.

In a ninth aspect of the present invention, it provides a method for producing a transgenic plant, comprising the steps of: regenerating a genetically engineered host cell according to the seventh aspect of the present invention into a plant, and the genetically engineered host cell is a plant cell.

In another preferred embodiment, the genetically engineered host cell is a ginseng cell.

In another preferred embodiment, the genetically engineered host cell is a Panax notoginseng cell.

It should be understood that, within the scope of the present invention, each technical feature of the present invention described above and in the following (as examples) may be combined with each other to form a new or preferred technical solution, which is not listed here due to space limitations.

### DESCRIPTION OF FIGURES

FIG. 1 panel A shows the expression shown by SDS-PAGE of glycosyltransferase genes GT29-32, GT29-33, and GT29-34 in E. coli; lane control represents total protein of lysate or lysis supernatant of empty vector recombinant pet28a; lane GT29-32 represents total protein or lysis supernatant of recombinant E. coli BL21-GT29-32; lane GT29-33 represents total protein or lysis supernatant of recombinant E. coli BL21-GT29-33; lane GT29-34 represents total protein of lysate or lysis supernatant of recombinant E. coli BL21-GT29-34; panel B shows the expression shown by Western Blot of glycosyltransferase genes GT29-32, GT29-33 and GT29-34 in E. coli; lane control represents total protein of lysate or lysis supernatant of empty vector recombinant pet28a; lane GT29-32 represents total protein of lysate or lysis supernatant of recombinant E. coli BL21-GT29-32; lane GT29-33 represents total protein of lysate or lysis supernatant of recombinant E. coli BL21-GT29-33; lane GT29-34 represents total protein of lysate or lysis supernatant of recombinant E. coli BL21-GT29-34.

FIG. 2 shows a TLC pattern of a transglycosyl reaction catalyzed by glycosyltransferases GT29-32, GT29-33 and GT29-34 with ginsenoside CK or Rd as a glycosyl acceptor and UDP-glucose or UDP-xylose as a glycosyl donor. Control represents the lysate supernatant of the pet28a empty vector recombinant as an enzyme solution; GT29-32, GT29-33 and GT29-34 respectively represent the lysate supernatants of recombinant E. coli BL21-GT29-32, BL21-GT29-33 and BL21-GT29-34 as an enzyme solution.

FIG. 3 shows an HPLC pattern of a transglycosyl reaction catalyzed by glycosyltransferases GT29-32, GT29-33 and GT29-34 with ginsenoside CK or Rd as a glycosyl acceptor and UDP-glucose as a glycosyl donor. Control represents the lysate supernatant of the pet28a empty vector recombinant as an enzyme solution; GT29-32, GT29-33 and GT29-34 respectively represent the lysate supernatants of recombinant E. coli BL21-GT29-32, BL21-GT29-33 and BL21-GT29-34 as an enzyme solution.

FIG. 4 shows an HPLC pattern of a transglycosyl reaction catalyzed by glycosyltransferases GT29-32, GT29-33, and GT29-34 with ginsenoside Rd as a glycosyl acceptor and UDP-xylose as a glycosyl donor. Control represents the lysate supernatant of the pet28a empty vector recombinant as an enzyme solution; GT29-32, GT29-33, and GT29-34 respectively represent the lysate supernatants of recombinant E. coli BL21-GT29-32, BL21-GT29-33 and BL21-GT29-34 as an enzyme solution.

FIG. 5 shows a TLC pattern of a transglycosyl reaction catalyzed by glycosyltransferases GT29-32, GT29-33 and GT29-34 with ginsenoside F1 as a glycosyl acceptor and UDP-glucose as a glycosyl donor. Control represents the lysate supernatant of the pet28a empty vector recombinant as an enzyme solution; GT29-32, GT29-33, and GT29-34 respectively represent the lysate supernatants of recombinant E. coli BL21-GT29-32, BL21-GT29-33 and BL21-GT29-34 as an enzyme solution.

FIG. 6 shows an HPLC pattern of a transglycosyl reaction catalyzed by glycosyltransferases GT29-32, GT29-33, and GT29-34 using ginsenoside F1 as a glycosyl acceptor and UDP-glucose as a glycosyl donor. Control represents the lysate supernatant of the pet28a empty vector recombinant as an enzyme solution; GT29-32, GT29-33, and GT29-34 respectively represent the lysate supernatants of recombinant E. coli BL21-GT29-32, BL21-GT29-33 and BL21-GT29-34 as an enzyme solution.

FIG. 7 shows a TLC pattern of a transglycosyl reaction catalyzed by the glycosyltransferases GT29-4, GT29-5, GT29-7, GT29-9, GT29-11, GT29-13, GT29-17, GT29-18, GT29-24 and GT29-25 with ginsenoside Rgl as a glycosyl acceptor and UDP-xylose as a glycosyl donor. Control represents the lysate supernatant of the pet28a empty vector recombinant as an enzyme solution; GT29-4, GT29-5, GT29-7, GT29-9, GT29-11, GT29-13, GT29-17, GT29-18, GT29-24 and GT29-25 respectively represent the lysate supernatants of recombinant E. coli BL21-GT29-4, BL21-GT29-5, BL21-GT29-7, BL21-GT29-9, BL21-GT29-11, BL21-GT29-13, BL21-GT29-17, BL21-GT29-18, BL21-GT29-24 and BL21-GT29-25 as an enzyme solution.

FIG. 8 shows an HPLC pattern of a transglycosyl reaction catalyzed by the glycosyltransferases GT29-4, GT29-5, GT29-7 and GT29-9 using ginsenoside Rgl as a glycosyl acceptor and UDP-xylose as a glycosyl donor. Control represents the lysate supernatant of the pet28a empty vector recombinant as an enzyme solution; GT29-4, GT29-5, GT29-7, and GT29-9, respectively represents the lysate supernatants of recombinant E. coli BL21-GT29-4, BL21-GT29-5, BL21-GT29-7 and BL21-GT29-9 as an enzyme solution.

FIG. 9 shows an HPLC pattern of a transglycosyl reaction catalyzed by the glycosyltransferases GT29-11, GT29-13, GT29-17, GT29-18, GT29-24 and GT29-25 with the ginsenoside Rgl as a glycosyl receptor and UDP-xylose as a glycosyl donor. Control represents the lysate supernatant of the pet28a empty vector recombinant as an enzyme solution; GT29-11, GT29-13, GT29-17, GT29-18, GT29-24 and GT29-25 respectively represents the lysate supernatants of recombinant E. coli BL21-GT29-11, BL21-GT29-13, BL21-GT29-17, BL21-GT29-18, BL21-GT29-24, and BL21-GT29-25 as an enzyme solution.

FIG. 10 shows Western blot detection for the protein expressions of glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, and PNUGT29-15. Control represents the lysate supernatant of the pet28a empty vector recombinant as an enzyme solution; PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 respectively represents the lysate supernatants of recombinant E. coli BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7, BL21-PNUGT29-8, BL21-PNUGT29-9, BL21-PNUGT29-14, and BL21-PNUGT29-15 as enzyme solution.

FIG. 11 shows a TLC pattern of a transglycosyl reaction catalyzed by the glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, and PNUGT29-15 with the ginsenoside Rd as a glycosyl acceptor and UDP-glucose as a glycosyl donor. Control represents the lysate supernatant of the pet28a empty vector recombinant as an enzyme solution; PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 respectively represents the lysate supernatants of recombinant E. coli BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7, BL21-PNUGT29-8, BL21-PNUGT29-9, BL21-PNUGT29-14, and BL21-PNUGT29-15 as an enzyme solution.

FIG. 12 shows a TLC pattern of a transglycosyl reaction catalyzed by the glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, and PNUGT29-15 with the ginsenoside CK as glycosyl acceptor and UDP-glucose as a glycosyl donor. Control represents the lysate supernatant of the pet28a empty vector recombinant as an enzyme solution; PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 respectively represents the lysate supernatants of recombinant E. coli BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7, BL21-PNUGT29-8, BL21-PNUGT29-9, BL21-PNUGT29-14, and BL21-PNUGT29-15 as an enzyme solution.

FIG. 13 shows a TLC pattern of a transglycosyl reaction catalyzed by the glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, and PNUGT29-15 with the ginsenoside Rh2 as a glycosyl acceptor and UDP-glucose as a glycosyl donor. Control represents the lysate supernatant of the pet28a empty vector recombinant as an enzyme solution; PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 respectively represents the lysate supernatants of recombinant E. coli BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7, BL21-PNUGT29-8, BL21-PNUGT29-9, BL21-PNUGT29-14, and BL21-PNUGT29-15 as enzyme solution.

FIG. 14 shows a TLC pattern of a transglycosyl reaction catalyzed by the glycosyltransferases GT29-36, GT29-36, GT29-42 and GT29-43 with ginsenoside Rd as a glycosyl acceptor and UDP-xylose as a glycosyl donor. GT29-36, GT29-36, GT29-42 and GT29-43 respectively represents the lysate supernatants of recombinant E. coli BL21-GT29-36, BL21-GT29-36, BL21-GT29-42 and BL21-GT29-43 as an enzyme solution.

FIG. 15 shows a TLC pattern of a transglycosyl reaction catalyzed by the glycosyltransferase GT29-45 and GT29-46 using ginsenoside Rh2 as a glycosyl acceptor and UDP-glucose as a glycosyl donor. Control represents the lysate supernatant of the pet28a empty vector recombinant as an enzyme solution; GT29-45 and GT29-46 respectively represents the lysate supernatant of the recombinant E. coli BL21-GT29-45 and BL21-GT29-46 as an enzyme solution.

FIG. 16 shows a TLC pattern of a transglycosyl reaction catalyzed by glycosyltransferases GT29-45 and GT29-46 using ginsenoside CK as a glycosyl acceptor and UDP-glucose as a glycosyl donor. Control represents the lysate supernatant of the pet28a empty vector recombinant as an enzyme solution; GT29-45 and GT29-46 respectively represents the lysate supernatant of the recombinant E. coli BL21-GT29-45 and BL21-GT29-46 as an enzyme solution.

FIG. 17 shows a TLC pattern of a transglycosyl reaction catalyzed by glycosyltransferase GT29-32 using ginsenoside CK as a glycosyl acceptor and UDP-arabinose as a glycosyl donor. Control represents the lysate supernatant of the pet28a empty vector recombinant as an enzyme solution; GT29-32 represents the lysate supernatant of the recombinant E. coli BL21-GT29-32 as an enzyme solution.

### DETAILED DESCRIPTION

After an extensive and in-depth study, the present inventors have firstly provided a new glycosyltransferase and the corresponding glycosyltransfer catalytic sites. Specifically, the glycosyltransferases GT29-32 (SEQ ID NO.: 4), GT29-33 (SEQ ID NO.: 6), GT29-34 (SEQ ID NO.: 8), GT29-4 (SEQ ID NO.: 12), GT29-5 (SEQ ID NO.: 14), GT29-7(SEQ ID NO.: 16), GT29-9 (SEQ ID NO.: 18), GT29-11 (SEQ ID NO.: 20), GT29-13 (SEQ ID NO.: 22), GT29-17 (SEQ ID NO.: 24), GT29-18 (SEQ ID NO.: 26), GT29-19 (SEQ ID NO.: 116), GT29-20 (SEQ ID NO:118), GT29-21 (SEQ ID NO:120), GT29-22 (SEQ ID NO: 122), GT29-23 (SEQ ID NO:124)), GT29-24 (SEQ ID NO.: 28), GT29-25 (SEQ ID NO.: 30), GT29-36 (SEQ ID NO.: 90), GT29-37 (SEQ ID NO.: 92), GT29-42 (SEQ ID NO.: 94), GT29-43 (SEQ ID NO.: 96), GT29-45 (SEQ ID NO.: 98), GT29-46 (SEQ ID NO.: 100), PNUGT29-1 (SEQ ID NO.: 39), PNUGT29-2 (SEQ ID NO.: 41), PNUGT29-3 (SEQ ID NO.: 43), PNUGT29-4 (SEQ ID NO.: 45), PNUGT29-5 ((SEQ ID NO.: 47), PNUGT29-6 (SEQ ID NO.: 49), PNUGT29-7 (SEQ ID NO.: 51), PNUGT29-8 (SEQ ID NO.: 53), PNUGT29-9(SEQ ID NO.: 55), PNUGT29-14 (SEQ ID NO.: 57), PNUGT29-15 (SEQ ID NO.: 59) can specifically and efficiently catalyze the hydroxyl glycosylation of the first glycosyl group on the C-20, C-6, or C3 position of a tetracyclic triterpene compound substrate or replace the original glycosyl group with a glycosyl group to extend the carbohydrate chain.

The glycosyltransferase of the present invention is particularly capable of converting ginsenosides CK, DMG, F2, Rd, F1, Rh1, and Rg1 to ginsenoside Rg3, ginsenoside Rd, ginsenoside Rb1, ginsenoside Rb3, saponin DMGG, saponin DMGX, gypenosides LXXV, gypenosides XVII, gypenosides XIII, gypenosides IX, notoginsenoside U and notoginsenoside R1 and notoginsenoside R2, notoginsenoside R3, 3-O-β-(D-xylopyranosyl)-β-(D-glucopyranosyl)-PPD 3-O-β-(D-xylopyranosyl)-β-(D-glucopyranosyl)-CK, 20-O-Glucosylginsenoside Rf and Ginsenoside F3.

### DEFINITION

As used herein, the terms “active polypeptide”, “polypeptide of the present invention and the derivative polypeptide thereof”, “the enzyme of the present invention” and “glycosyltransferase” can be used interchangeably and all refer to GT29-32 (SEQ ID NO.: 4), GT29-33 (SEQ ID NO.: 6), GT29-34 (SEQ ID NO.: 8), GT29-4 (SEQ ID NO.: 12), GT29-5 (SEQ ID NO.: 14), GT29-7 (SEQ ID NO.: 16), GT29-9 (SEQ ID NO.: 18), GT29-11 (SEQ ID NO.: 20), GT29-13 (SEQ ID NO.: 22), GT29-17 (SEQ ID NO.: 24), GT29-18 (SEQ ID NO.: 26), GT29-19 (SEQ ID NO.: 116), GT29-20 (SEQ ID NO.: 118), GT29-21 (SEQ ID NO: 120), GT29-22 (SEQ ID NO:122), GT29-23 (SEQ ID NO:124), GT29-24 (SEQ ID NO.: 28), GT29-25 (SEQ ID NO.: 30), GT29-36 (SEQ ID NO.: 90), GT29-37 (SEQ ID NO.: 92), GT29-42 (SEQ ID NO.: 94), GT29-43 (SEQ ID NO.: 96), GT29-45 (SEQ ID NO.: 98), GT29-46 (SEQ ID NO.: 100), PNUGT29-1 (SEQ ID NO.: 39), PNUGT29-2 (SEQ ID NO.: 41), PNUGT29-3 (SEQ ID NO.: 43), PNUGT29-4 (SEQ ID NO.: 45), PNUGT29-5 (SEQ ID NO.: 47), PNUGT29-6 (SEQ ID NO.: 49), PNUGT29-7 (SEQ ID NO.: 51), PNUGT29-8(SEQ ID NO.: 53), PNUGT29-9 (SEQ ID NO.: 55), PNUGT29-14 (SEQ ID NO: 57), PNUGT29-15 (SEQ ID NO.: 59) polypeptides and the derivative polypeptides thereof.

As used herein, “the isolated polypeptide” or “active polypeptide” means that the polypeptide is substantially free of other proteins, lipids, carbohydrates, or other substances with which it is naturally associated. Those skilled in the art can purify the polypeptide using standard protein purification techniques. Substantially pure polypeptides can form a single main band on a non-reduced polyacrylamide gel. The purity of the polypeptide can be further analyzed using the amino acid sequence.

The active polypeptide of the present invention may be a recombinant polypeptide, a natural polypeptide, or a synthetic polypeptide. The polypeptides of the present invention may be naturally purified products or chemically synthesized products, or produced from prokaryotic or eukaryotic hosts (e.g., bacteria, yeast, plants) using recombinant techniques. Depending on the host used in the recombinant production protocol, the polypeptides of the present invention may be glycosylated or may be non-glycosylated. The polypeptides of the present invention may also include or exclude the starting methionine residue.

The present invention further provides fragments, derivatives and analogs of the polypeptides. As used herein, the terms “fragment”, “derivative” and “analog” refer to a polypeptide that substantially retains the same biological function or activity of the polypeptide.

The polypeptide fragment, derivative or analog of the present invention may be (i) a polypeptide having one or more conservative or non-conservative amino acid residues (preferably conservative amino acid residues) substituted, and such substituted amino acid residues may or may not be encoded by the genetic code, or (ii) a polypeptide having a substituent group in one or more amino acid residues, or (iii) a polypeptide formed by fusion of a mature polypeptide with another compound, such as a compound that extends the half-life of the polypeptide, such as polyethylene glycol, or (iv) a polypeptide formed by fusing an additional amino acid sequence to this polypeptide sequence (such as a leader sequence or a secreted sequence or a sequence or protease sequence used to purify this polypeptide, or a fusion protein formed with an antigen IgG fragment). In accordance with the teachings herein, these fragments, derivatives, and analogs are within the scope of those skilled in the art.

The active polypeptide of the present invention has glycosyltransferase activity and can catalyze one or more of the following reactions:

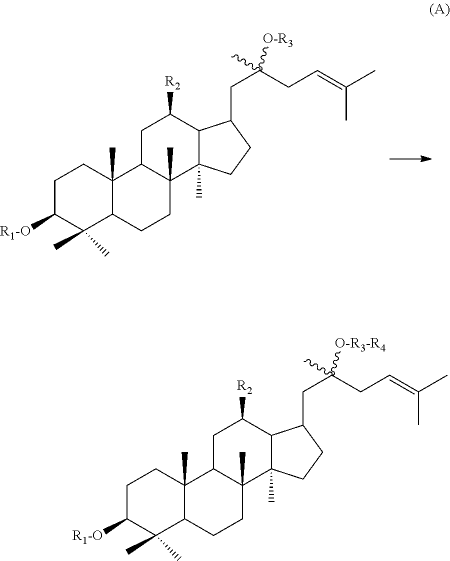

Compound of Formula (I) Compound of Formula (II);
wherein R1 is H, a monosaccharide glycosyl or a polysaccharide glycosyl; R2 is H or OH; R3 is a monosaccharide glycosyl; R4 is a monosaccharide glycosyl, and the polypeptide is selected from SEQ ID NO: 4, 6, 8, 39, 41, 43, 45, 47, 49, 51, 53, 55, 57, 59, 98, 100, 116, 118, 120, 122, and 124 and a derivative polypeptide thereof.

In another preferred embodiment, the monosaccharide comprises glucose (Glc), rhamnose (Rha), acetylg

In another preferred embodiment, the polysaccharide comprises Glc(2-1) Glc, Glc(6-1) Glc, Glc(6) Ac, Glc(2-1) Rha, Glc(6-1) Arap, Glc(6-1) Xyl, Glc(6-1) Araf, Glc(3-1) Glc(3-1), Glc(2-1) Glu (6) Ac, Glc(6-1) Arap (4-1) Xyl, Glc(6-1) Arap (2-1) Xyl, or Glc(6-1) Arap (3-1) Xyl and other polysaccharides composed of 2-4 monosaccharides.

The R1-R4 substituted compounds are shown in the table below:

| substrate | R1 | R2 | R3 | R4 | product |
|---|---|---|---|---|---|
| CK | H | OH | Glc | Glc | Gypenosides |
| | | | | | LXXV |
| DMG | H | H | Glc | Glc | DMGG |
| F2 | Glc | OH | Glc | Glc | Gypenosides |
| | | | | | XVII |
| Rd | Glc(2-1)Glc | OH | Glc | Glc | Rbl |
| CK | H | OH | Glc | Xyl | Gypenosides |
| | | | | | XIII |
| DMG | H | H | Glc | Xyl | DMGX |
| F2 | Glc | OH | Glc | Xyl | Gypenosides |
| | | | | | IX |
| Rd | Glc(2-1)Glc | OH | Glc | Xyl | Rb3 |
| CK | H | OH | Glc | Arabinose | Ginsenoside F3 |

that is, when R1 is H, R2 is OH, and R3 is a glucosyl, the compound of formula (I) is ginsenoside CK (CK);
when R1 and R2 are both H, and R3 is a glucosyl, the compound of Formula (I) is ginsenoside DMG;
when R1 is a glucosyl, R2 is OH, and R3 is a glucosyl, the compound of Formula (I) is ginsenoside F2 (F2); or
when R1 is two glucosyls (Glc(2-1) Glc), R2 is OH, and R3 is a glucosyl, the compound of Formula (I) is ginsenoside Rd;

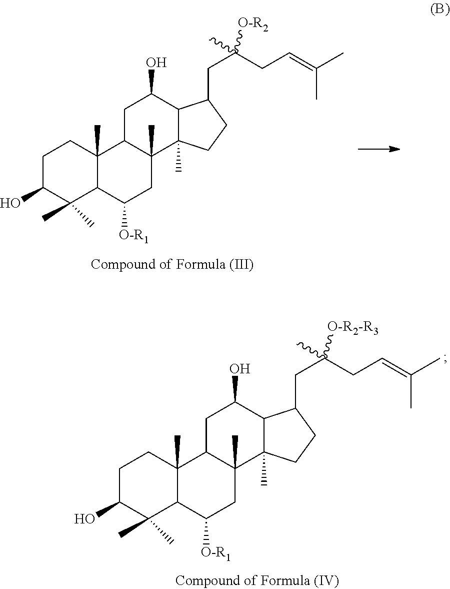

wherein R1 is H, a glycosyl or a polysaccharide glycosyl, R2 is a glycosyl, R3 is a glycosyl, the polypeptide is selected from SEQ ID NOs.: 4 and a derivative polypeptide thereof;

The R1-R3 substituted compounds are shown in the table below:

| substrate | R1 | R2 | R3 | product |
|---|---|---|---|---|
| Fl | H | Glc | Glc | Notoginsenoside U |
| Rgl | Glc | Glc | Glc | Notoginsenoside R3 |

that is, when R1 is H and R2 is a glucosyl, the compound of Formula (III) is ginsenoside F1 (F1); or when R1 and R2 are glucosyls, the compound of Formula (III) is ginsenoside Rg1 (Rg1);

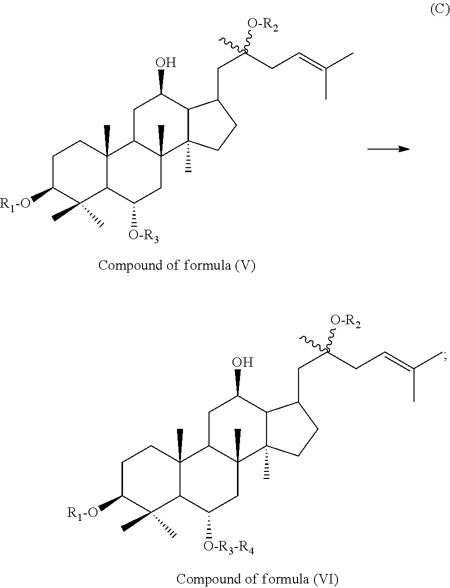

wherein R1 and R2 are H or glycosyls, and R3 and R4 are glycosyls. The polypeptide is selected from SEQ ID NOs.: 12, 14, 16, 18, 20, 22, 24, 26, 28, 30 or a derivative polypeptide thereof;

The R1-R4 substituted compounds are shown in the table below:

| substrate | R1 | R2 | R3 | R4 | product |
|---|---|---|---|---|---|
| Rgl | H | Glc | Glc | Xyl | Notoginsenoside R1 |
| Rgl | H | Glc | Glc | Glc | 20-O-Glucosylginsenoside |
| | | | | | Rf |
| Rhl | H | H | Glc | Xyl | Notoginsenoside R2 |
| Rhl | H | H | Glc | Glc | Ginsenoside Rf |

that is, when R1 is H and both R2 and R3 are glucosyls, the compound of Formula (V) is ginsenoside Rg1;
when R1 and R2 are H, and R3 is glucosyl, the compound of Formula (V) is ginsenoside Rh1.

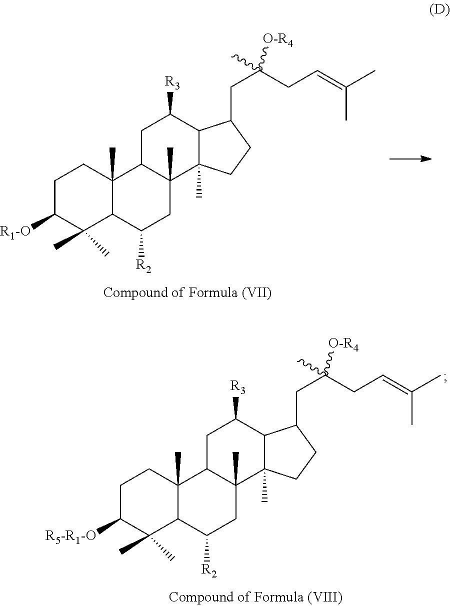

wherein R1 is a glycosyl; R2 and R3 are OH or H; R4 is a glycosyl or H; R5 is a glycosyl, R5-R1-O is a glycosyl derived from the first glycosyl of C3; and the polypeptide is selected from SEQ ID NOs.: 26, 28, 30, 39, 41, 43, 45, 47, 49, 51, 53, 55, 57, 59, 98, 100, 116, 118, 120, 122, and 124 and a derivative polypeptide thereof;

| substrate | R1 | R2 | R3 | R4 | R5 | product |
|---|---|---|---|---|---|---|
| Rh2 | Glc | H | OH | H | Glc | Rg3 |
| F2 | Glc | H | OH | Glc | Glc | Rd |
| Gypenoside | Glc | H | OH | Glc(6,1)Glc | Glc | Rb1 |
| XVII | | | | | | |
| Gypenoside | Glc | H | OH | Glc(6,1)xyl | Glc | Rb3 |
| IX | | | | | | |

that is, when R1 is a glucosyl; R2 is H, R3 is OH, R4 is H, and the compound of Formula (VII) is Rh2;
R1 is a glucosyl; R2 is H, R3 is OH, R4 is a glucosyl, and the compound of Formula (VII) is F2;
R1 is a glucosyl; R2 is H, R3 is OH, R4 is two glucosyl groups, and the compound of Formula (VII) is Gypenoside XVII;

R1 is a glucosyl; R2 is H, R3 is OH, R4 is a glucosyl extended with a xylose, the compound of Formula (VII) is Gypenoside IX;

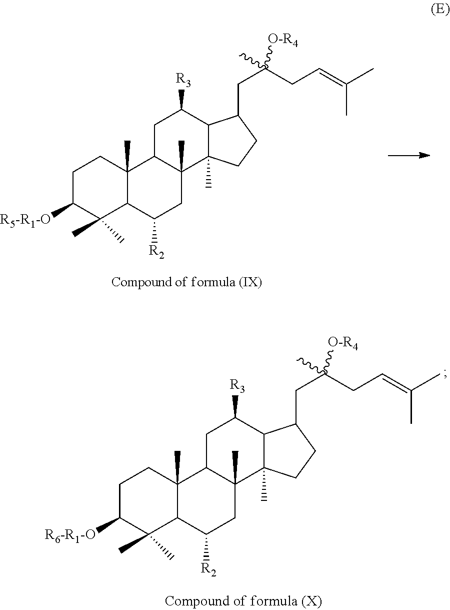

wherein R1 is a glycosyl; R2 and R3 are OH or H; R4 is a glycosyl or H; R5 is a glycosyl and R5-R1-O is a glycosyl derived from the first glycosyl of C3; R6 is a glycosyl and R6-R1-O is a glycosyl derived from the first glycosyl of C3, and the polypeptide is selected from SEQ ID NOs.: 41, 45, 90, 92, 94, and 96 and a derivative polypeptide thereof;
R1 is two glucosyl groups, R2 is H, R3 is OH, R4 is H, and the compound of Formula (IX) is Rg3.
R1 is two glucosyl groups, R2 is H, R3 is OH, R4 is glucosyl, and the compound of Formula (IX) is Rd.

The preferred sequence of the polypeptide is as shown in SEQ ID NO.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124, and the term also includes polypeptide variants and the derived polypeptides that have the same function as the indicated polypeptides of SEQ ID NO.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124. These variant forms include (but are not limited to): one or more (usually 1-50, preferably 1-30, more preferably 1-20, most preferably 1-10) amino acid deletions, insertions and/or substitutions and the addition of one or several (usually within 20, preferably within 10, more preferably within 5) amino acids at the C-terminus and/or N-terminus. For example, in the art, the substitution of amino acids with similar or close properties usually does not change the function of the protein. As another example, adding one or several amino acids to the C-terminus and/or N-terminus usually does not change the function of the protein. The term also includes active fragments and active derivatives of the polypeptides of the present invention. The present invention also provides analogues of the polypeptides. The difference between these analogues and the natural polypeptide of the present invention may be a difference in amino acid sequence, a difference in the modification form that does not affect the sequence, or both. These polypeptides include natural or induced genetic variants. Induced variants can be obtained by various techniques, such as random mutagenesis by radiation or exposure to mutagen, or by site-directed mutagenesis or other known molecular biology techniques. Analogs also include analogs with residues different from natural L-amino acids (such as D-amino acids), and analogs with non-naturally occurring or synthetic amino acids (such as (3, y-amino acids). It should be understood that the polypeptide of the present invention is not limited to the representative polypeptides exemplified above.

Modified (usually without changing the primary structure) forms include: in vivo or in vitro chemically derived forms of the polypeptide such as acetylation or carboxylation. Modifications also include glycosylation, such as those produced by glycosylation modification during the synthesis and processing of polypeptides or during further processing steps. This modification can be accomplished by exposing the polypeptide to an enzyme that performs glycosylation (such as mammalian glycosylation or deglycosylation enzymes). Modified forms also include sequences with phosphorylated amino acid residues (e.g., phosphotyrosine, phosphoserine, phosphothreonine). Also included are peptides that have been modified to improve their proteolytic resistance or optimize their solubility.

The amino or carboxyl terminus of GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 or PNUGT29-15 protein of the present invention may also contain one or more polypeptide fragments as protein tags. Any suitable tags can be used in the present invention. For example, the tags may be FLAG, HA, HAI, c-Myc, Poly-His, Poly-Arg, Strep-TagII, AU1, EE, T7, 4A6, c, B, gE, and Tyl. These tags can be used to purify proteins. Table 1 lists some of the commercially available tags.

TABLE 1

| | number of |
|---|---|
| tag | residues |
| Poly-Arg | 5-6 (usually 5) |
| Poly-His | 2-10 (usually 6) |
| FLAG | 8 |
| Strep-TagII | 8 |
| C-myc GST | 10 |
| | 220 |

In order to make the translated protein secreted and expressed (such as secreted out of the cell), a signal peptide sequence, such as pel signal peptide and the like can be added to the amino terminus of GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-9, PNUGT29-14or PNUGT29-15. The signal peptide can be cleaved during the secretion of the polypeptide from the cell.

The polynucleotide of the present invention may be in the form of DNA or RNA. DNA form includes cDNA, genomic DNA, or synthetic DNA. DNA can be single-stranded or double-stranded. DNA can be a coding strand or a non-coding strand. The coding region sequence encoding the mature polypeptide can be the same with the coding region sequence as shown in SEQ ID NO.: 3, 5, 7, 11, 13, 15, 17, 19, 21, 23, 25, 27, 29, 38, 40, 42, 44, 46, 48, 50, 52, 54, 56, 58, 89, 91, 93, 95, 97, 99, 115, 117, 119, 121, or 123 or degenerate variants. As used herein, “degenerate variant” in the present invention refers to a nucleic acid sequence encoding the protein having SEQ ID NO.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124, but differing in the coding region sequences as shown in SEQ ID NO: 3, 5, 7, 11, 13, 15, 17, 19, 21, 23, 25, 27, 29, 38, 40, 42, 44, 46, 48, 50, 52, 54, 56, 58, 89, 91, 93, 95, 97, 99, 115, 117, 119, 121, or 123, respectively.

Polynucleotides encoding mature polypeptides of SEQ ID NO.: 4, 6, 8, 12, 14, 16, 18, 20, 22, 24, 26, 28, 30, 39, 41, 43, 45, 47, 49, 51, 53, 55, 57, 59, 90, 92, 94,96, 98, 100, 116, 118, 120, 122, or 124 include: coding sequences encoding mature polypeptides only; coding sequences encoding mature polypeptides and various additional coding sequences; mature polypeptide coding sequences (and optional additional coding sequences) and non-coding sequences.

The term “polynucleotide encoding a polypeptide” may include a polynucleotide encoding the polypeptide, or a polynucleotide further including additional coding and/or non-coding sequences.

The present invention also relates to variants of the aforementioned polynucleotides, which encode fragments, analogues and derivatives of polypeptides or polypeptides having the same amino acid sequence as the present invention. This polynucleotide variant may be a naturally occurring allelic variant or a non-naturally occurring variant. These nucleotide variants include substitution variants, deletion variants and insertion variants. As known in the art, an allelic variant is a form of substitution of a polynucleotide. It may be a substitution, deletion, or insertion of one or more nucleotides, but it will not substantially change the function of the polypeptide encoded.

The present invention also relates to polynucleotides that hybridize to the above-mentioned sequences and have at least 50%, preferably at least 70%, more preferably at least 80%, 85%, 90%, 95% identity between the two sequences. The present invention particularly relates to polynucleotides that can hybridize to the polynucleotides of the present invention under stringent conditions (or stringent conditions). In the present invention, “stringent conditions” means: (1) hybridization and elution at a lower ionic strength and higher temperature, such as 0.2 x SSC, 0.1% SDS, 60° C.; or (2) added with denaturing agents during hybridization, such as 50% (v/v) formamide, 0.1% calf serum/0.1% Ficoll, 42° C., etc.; or (3) hybridization only when the identity between the two sequences is at least 90%, more preferably at least 95%. Furthermore, the polypeptides encoded by the hybridizable polynucleotides have the same biological function and activity as the mature polypeptides as shown in SEQ ID NO.: 4, 6, 8, 12, 14, 16, 18, 20, 22, 24, 26, 28, 30, 39, 41, 43, 45, 47, 49, 51, 53, 55, 57, 59, 90, 92, 94, 96, 98, 100, 116, 118, 120, 122, or 124.

The present invention also relates to a nucleic acid fragment hybridized to the aforementioned sequences. As used herein, “nucleic acid fragment” contains at least 15 nucleotides in length, preferably at least 30 nucleotides, more preferably at least 50 nucleotides, and most preferably at least 100 nucleotides or more. Nucleic acid fragments can be used in nucleic acid amplification techniques (such as PCR) to determine and/or isolate polynucleotides encoding GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 or PNUGT29-15 protein.

The polypeptide and polynucleotide in the present invention are preferably provided in an isolated form, and are more preferably purified to homogeneity.

A full-length nucleotide sequence or fragment thereof of GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 or PNUGT29-15 of the present invention can usually be obtained by PCR amplification method, recombination method or artificial synthesis method. For the PCR amplification method, primers can be designed according to the relevant nucleotide sequence disclosed in the present invention, especially the open reading frame sequence, and a commercially available cDNA library or cDNA library prepared according to conventional methods known to those skilled in the art is used as a 25 template to amplify and obtain the relevant sequences. When the sequence is long, it is often necessary to perform two or more PCR amplifications, and then splice the amplified fragments together in the correct order.

Once the relevant sequence is obtained, the relevant sequence can be obtained in large quantities by the recombination method. This is usually done by cloning it into a vector, then transferring it into cells, and then isolating and obtaining the relevant sequence from the proliferated host cells by conventional methods.

In addition, artificial synthetic methods can be used to synthesize the relevant sequences, especially when the length of the fragments is short. Generally, a long sequence can be obtained by synthesizing multiple small fragments and then connecting them.

At present, the DNA sequence encoding the protein (or fragment or derivative thereof) of the present invention can be obtained completely by chemical synthesis. This DNA sequence can then be introduced into various existing DNA molecules (or such as vectors) and cells known in the art. In addition, mutations can also be introduced into the protein sequence of the present invention by chemical synthesis.

The method of amplifying DNA/RNA using PCR technology is preferably used to obtain the gene of the present invention. Especially when it is difficult to obtain full-length cDNA from the library, the RACE method (RACE-cDNA terminal rapid amplification method) can be preferably used, and the primers used for PCR can be appropriately selected based on the sequence information of the present invention disclosed herein, and can be synthesized by conventional methods. The amplified DNA/RNA fragments can be separated and purified by conventional methods such as gel electrophoresis.

The present invention also relates to a vector comprising the polynucleotide of the present invention, and a host cell produced by genetic engineering using the vector of the present invention or the protein coding sequence of GT29-32, GT29-33, GT29-34, GT29-4, GT29-5, GT29-7, GT29-9, GT29-11, GT29-13, GT29-17, GT29-18, GT29-19, GT29-20, GT29-21,

GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 or PNUGT29-15, and the method of producing the polypeptide of the present invention by recombinant technology.

Through the conventional recombinant DNA technology, the polynucleotide sequence of the present invention can be used to express or produce recombinant GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 or PNUGT29-15 polypeptide.

Generally speaking, there are the following steps:

(1) transforming or transducing a suitable host cell with a polynucleotide (or a variant) encoding a polypeptide of GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-5, PNUGT29-6, PNUGT29-7, PNUGT29-8, PNUGT29-9. PNUGT29-14 or PNUGT29-15 of the present invention, or with a recombinant expression vector containing the polynucleotide;

(2). culturing a host cell in a suitable medium;

(3). isolating and purifing proteins from culture medium or cells.

In the present invention, polynucleotide sequences of GT29-32, GT29-33, GT29-34, GT29-4, GT29-5, GT29-7, GT29-9, GT29-11, GT29-13, GT29-17, GT29-18, GT29 19, GT29-20, GT29-21, GT29-22, GT29-23, GT29-24, GT29-25, GT29-32, GT29-33, GT29-34, GT29-36, GT29-37, GT29-42, GT29-43, GT29-45, GT29-46, PNUGT29-1, PNUGT29-2, PNUGT29-3,

PNUGT29-4, PNUGT29-9, PNUGT29-14 or PNUGT29-15 can be inserted into recombinant expression vectors. The term “recombinant expression vector” refers to a bacterial plasmid, bacteriophage, a yeast plasmid, plant cell virus, mammalian cell virus such as adenovirus, retrovirus, or other vectors well known in the art. As long as it can replicate and stabilize in the host, any plasmid and vector can be used. An important feature of expression vectors is that they usually contain an origin of replication, a promoter, a marker gene and a translation control element.

Methods well known to those skilled in the art can be used to construct expression vectors containing GT29-32, GT29-33, GT29-34, GT29-4, GT29-5, GT29-7, GT29-9, GT29-11, GT29-13, GT29-17, GT29-18, GT29-19, GT29-20, GT29-21, GT29-22, GT29-23, GT29-24,

GT29-25, GT29-32, GT29-33, GT29-34, GT29-36, GT29-37, GT29-42, GT29-43, GT29-45, GT29-46, PNUGT29-1, PNUGT29-2, PNUGT29-3, PNUGT29-4, PNUGT29-9, PNUGT29-14or PNUGT29-15 encoding DNA sequences and appropriate transcription/translation control signals. These methods include in vitro recombinant DNA technology, DNA synthesis technology, in vivo recombinant technology and the like. The DNA sequence can be effectively linked to an appropriate promoter in an expression vector to guide mRNA synthesis. Representative examples of these promoters are: lac or trp promoters of E. coli; λ phage PL promoters; eukaryotic promoters including CMV immediate early promoters, HSV thymidine kinase promoters, early and late SV40 promoters, retroviral LTRs and other known promoters that control gene expression in prokaryotic or eukaryotic cells or their viruses. The expression vector also includes a ribosome binding site for translation initiation and a transcription terminator.

In addition, the expression vector preferably contains one or more selectable marker genes to provide phenotypic traits for selection of transformed host cells, such as dihydrofolate reductase, neomycin resistance, and green fluorescent protein (GFP) for eukaryotic cell culture, or tetracycline or ampicillin resistance for E. coli.

Vectors containing the appropriate DNA sequences and appropriate promoters or control sequences as described above can be used to transform appropriate host cells so that they can express proteins.

The host cell may be a prokaryotic cell, such as a bacterial cell; or a lower eukaryotic cell, such as a yeast cell; or a higher eukaryotic cell, such as a mammalian cell. Representative examples are: E. coli, Streptomyces; bacterial cells of Salmonella typhimurium; fungal cells such as yeast; plant cells; insect cells of Drosophila S2 or Sf9; animal cells such as CHO, COS, 293 cells, or Bowes melanoma cells and the like.

When the polynucleotide of the present invention is expressed in higher eukaryotic cells, if an enhancer sequence is inserted into the vector, transcription will be enhanced. Enhancers are cis-acting factors of DNA, usually about 10 to 300 base pairs, which act on the promoter to enhance gene transcription. Examples include 100 to 270 base pair of SV40 enhancers on the late side of the replication start point, polyoma enhancers on the late side of the replication start point, and adenovirus enhancers.

Those of ordinary skill in the art know how to select appropriate vectors, promoters, enhancers and host cells.

Transformation of host cells with recombinant DNA can be performed using conventional techniques well known to those skilled in the art. When the host is a prokaryotic organism such as E. coli, competent cells that can absorb DNA can be harvested after the exponential growth phase and treated with the CaCl₂ method. The procedures used are well known in the art. Another method is to use MgCl₂. If necessary, transformation can also be carried out by electroporation. When the host is a eukaryote, the following DNA transfection methods can be used: calcium phosphate co-precipitation method, conventional mechanical methods such as microinjection, electroporation, liposome packaging, etc.

The obtained transformant can be cultured by a conventional method and express the polypeptide encoded by the gene of the present invention. Depending on the host cell used, the medium used in the culture can be selected from various conventional mediums. The cultivation is carried out under conditions suitable for the growth of host cells. When the host cell grows to an appropriate cell density, the selected promoter is induced by an appropriate method (such as temperature conversion or chemical induction), and the cell is cultured for a period of time.

The recombinant polypeptide in the above method may be expressed in a cell, on a cell membrane, or secreted out of the cell. If necessary, the recombinant protein can be isolated and purified by various separation methods using its physical, chemical and other characteristics. These methods are well known to those skilled in the art. Examples of these methods include, but are not limited to: conventional renaturation treatment, treatment with protein precipitation agent (salting out method), centrifugation, bacteria disruption through osmosis, ultra-treatment, ultra-centrifugation, molecular sieve chromatography (gel filtration), adsorption chromatography, ion exchange chromatography, high performance liquid chromatography (HPLC) and various other liquid chromatography techniques and combinations of these methods.

### Use

The active polypeptide or glycosyltransferase involved in the present invention can be used to artificially synthesize known ginsenosides and new ginsenosides and the derivatives thereof, and can convert CK, DMG, F2, Rd, F1, Rh1 and Rg1 into ginsenoside Rg3, ginsenoside Rd, ginsenoside Rb1, ginsenoside Rb3, saponin DMGG, saponin DMGX, Gypenosides LXXV, Gypenosides XVII, Gypenosides XIII, Gypenosides IX, notoginsenoside U and, notoginsenoside R1, and notoginsenoside R2, notoginsenoside R3, 3-O-β-(D-xylopyranosyl)-β-(D-glucopyranosyl)-PPD; 3-O-β-(D-xylopyranosyl)-β-(D-glucopyranosyl) -CK, 20-O-Glucosylginsenoside Rf and Ginsenoside F3.

The main advantages of the invention:

(1) The glycosyltransferase of the present invention can specifically and efficiently transfer a glycosyl or replace a glycosyl on the first glycosyl on the C-20 position/or the first glycosyl on the C-6 or C-3 position of the substrate of the tetracyclic triterpene compound to extend the carbohydrate chain;

(2) The glycosyltransferase of the present invention is particularly capable of converting CK, DMG, F2, Rd, F1, Rhl and Rg1 into active ginsenoside Rg3, ginsenoside Rd, ginsenoside Rb1, ginsenoside Rb3, saponin DMGG, Saponin DMGX, Gypenosides LXXV, Gypenosides XVII, Gypenosides XIII, Gypenosides IX, notoginsenoside U, notoginsenoside R1, and notoginsenoside R2, notoginsenoside R3, 3-O-β-(D-xylopyranosyl)-β-(D-glucopyranosyl)-PPD; 3-O-β-(D-xylopyranosyl)-β-(D-glucopyranosyl)-CK, 20-O-Glucosylginsenoside Rf and Ginsenoside F3.

(3) Ginsenoside Rb1 has the effect of protecting nerve cells and anti-inflammatory and antioxidant; and ginsenoside Rb3 has the effect of alleviating myocardial ischemia and anti-depression. Notoginsenoside R1 is the main active ingredient of notoginsenoside with anti-inflammatory effects. Notoginsenoside R2 has a neuroprotective effect.

### Example 1

### Isolation of Ginseng Glycosyltransferase and the Coding Gene Thereof

Ginseng RNA was extracted and reverse transcription was performed to obtain ginseng cDNA. PCR amplification was performed using primer pair 1 (SEQ ID NO: 1 and SEQ ID NO: 2) or primer pair 2 (SEQ ID NO: 9 and SEQ ID NO: 10) or primer pair 3 (SEQ ID NO: 113 and SEQ ID NO: 114) using this cDNA as a template to obtain a 1.4-1.5 kb amplification product. The high-fidelity KOD DNA polymerase from Bao Bioengineering Co., Ltd. was used as the DNA polymerase. PCR products were detected by agarose gel electrophoresis.

The target DNA band was cut off under UV irradiation. Then the Axygen Gel Extraction Kit (AEYGEN) was used to recover DNA from the agarose gel, that is, the
amplified DNA fragment. After A was added at the end of this DNA fragment using rTaq DNA polymerase from Bao Bioengineering Co., Ltd., it was ligated with the commercially available cloning vector pMD18-T Vector, and the ligation product was transformed into commercially available E. coli EPI300 competent cells. The transformed E. coli solution was coated on LB plates supplemented with AMP 50 μg/mL, IPTG 0.5 mM, X-Gal 25 μg/mL, and the recombinant clone was further verified by PCR and enzyme digestion. Several clones were selected and the recombinant plasmids were extracted and sequenced to obtain 29 different nucleic acid sequences, named GT29-32 (SEQ ID NO.: 3), GT29-33 (SEQ ID NO.: 5), GT29-34 (SEQ ID NO.: 7), GT29-4 (SEQ ID NO.: 11), GT29-5 (SEQ ID NO.: 13), GT29-7 (SEQ ID NO.: 15), GT29-9 (SEQ ID NO.: 17), GT29-11 (SEQ ID NO.: 19), GT29-13 (SEQ ID NO.: 21), GT29-17 (SEQ ID NO.: 23), GT29-18 (SEQ ID NO.: 25), GT29-19 (SEQ ID NO.: 116), GT29-20 (SEQ ID NO.: 118), GT29-21 (SEQ ID NO.: 120), GT29-22 (SEQ ID NO.: 122)), GT29-23 (SEQ ID NO.: 124), GT29-24 (SEQ ID NO.: 27), GT29-25 (SEQ ID NO.: 29), GT29-36 (SEQ ID NO.: 89), GT29-37 (SEQ ID NO.: 91), GT29-42 (SEQ ID NO.: 93), GT29-42 (SEQ ID NO.: 95), GT29-45 (SEQ ID NO.: 97) and GT29-46 (SEQ ID NO.: 99), respectively. Using BESTORF software to find ORF. Through sequence alignment, it was found that the extension products all have the conserved functional domain of glycosyltransferase family 1, indicating that it is a glycosyltransferase gene.

GT29-32: The glycosyltransferase gene GT29-32 encodes a protein GT29-32 containing 442 amino acids and has the amino acid sequence as shown in SEQ ID NO: 4 in the sequence listing. The theoretical molecular weight of this protein is predicted to be 49.2 kDa by software, and the isoelectric point pI is 6.09. The amino acid sequence identity between the glycosyltransferase and the functionally identified glycosyltransferase UGTPg29 (Genbank accession AKA44579.1) is 92%.

GT29-33: The glycosyltransferase gene GT29-33 encodes a protein GT29-33 containing 448 amino acids with the amino acid sequence as shown in SEQ ID NO: 6 in the sequence listing. The theoretical molecular weight of the protein is predicted to be 50.0 kDa by software, and the isoelectric point pI is 6.77. The amino acid sequence identity between the glycosyltransferase and the functionally identified glycosyltransferase UGTPg29 is 90%.

GT29-34: The glycosyltransferase gene GT29-34 encodes a protein GT29-34 containing 446 amino acids and has the amino acid sequence as shown in SEQ ID NO: 8in the sequence listing. The theoretical molecular weight of the protein is predicted to be 49.7 kDa by software, and the isoelectric point pI is 6.23. The amino acid sequence identity between the glycosyltransferase and the functionally identified glycosyltransferase UGTPg29 is 90%.

GT29-4: The glycosyltransferase gene GT29-4 encodes a protein GT29-4 containing 446 amino acids with the amino acid sequence as shown in SEQ ID NO: 12in the sequence listing. The theoretical molecular weight of the protein is predicted to be 49.8 kDa by software, and the isoelectric point pI is 5.63. The amino acid sequence identity between the glycosyltransferase and the functionally identified glycosyltransferase UGTPg29 is 92%.

GT29-5: The glycosyltransferase gene GT29-5 encodes a protein GT29-5 containing 446 amino acids with the amino acid sequence as shown in SEQ ID NO: 14 in the sequence listing. The theoretical molecular weight of the protein is predicted to be 49.7 kDa by software, and the isoelectric point pI is 5.93. The amino acid sequence identity between the glycosyltransferase and the functionally identified glycosyltransferase UGTPg29 is 93%.

GT29-7: The glycosyltransferase gene GT29-7 encodes protein GT29-7 containing 446 amino acids with the amino acid sequence as shown in SEQ ID NO: 16 in the sequence listing. The theoretical molecular weight of the protein is predicted to be 49.8 kDa by software, and the isoelectric point pI is 5.8. The amino acid sequence
identity between the glycosyltransferase and the functionally identified glycosyltransferase UGTPg29 is 92%.

GT29-9: The glycosyltransferase gene GT29-9 encodes a protein GT29-9 containing 446 amino acids with the amino acid sequence as shown in SEQ ID NO: 18 in the sequence listing. The theoretical molecular weight of this protein is predicted to be 49.8 kDa by software, and the isoelectric point pI is 5.93. The amino acid sequence identity between the glycosyltransferase and the functionally identified glycosyltransferase UGTPg29 is 92%.

GT29-11: The glycosyltransferase gene GT29-11 encodes a protein GT29-11 containing 446 amino acids with the amino acid sequence as shown in SEQ ID NO: 20 in the sequence listing. The theoretical molecular weight of the protein is predicted to be 49.9 kDa by software, and the isoelectric point pI is 5.90. The amino acid sequence identity between the glycosyltransferase and the functionally identified glycosyltransferase UGTPg29 is 91%.

GT29-13: The glycosyltransferase gene GT29-13 encodes a protein GT29-13 containing 446 amino acids with the amino acid sequence as shown in SEQ ID NO: 22 in the sequence listing. The theoretical molecular weight of the protein is predicted to be 49.9 kDa by software, and the isoelectric point pI is 5.93. The amino acid sequence identity between the glycosyltransferase and the functionally identified glycosyltransferase UGTPg29 is 91%.

GT29-17: The glycosyltransferase gene GT29-17 encodes a protein GT29-17 containing 442 amino acids with the amino acid sequence as shown in SEQ ID NO: 24 in the sequence listing. The theoretical molecular weight of the protein is predicted to be 49.3 kDa by software, and the isoelectric point pI is 5.35. The amino acid sequence identity between the glycosyltransferase and the functionally identified glycosyltransferase UGTPg29 is 93%.

GT29-18: The glycosyltransferase gene GT29-18 encodes a protein GT29-18 containing 446 amino acids with the amino acid sequence as shown in SEQ ID NO: 26 in the sequence listing. The theoretical molecular weight of the protein is predicted to be 49.9 kDa by software, and the isoelectric point pI is 5.93. The amino acid sequence identity between the glycosyltransferase and the functionally identified glycosyltransferase UGTPg29 is 91%.

GT29-24: The glycosyltransferase gene GT29-24 encodes a protein GT29-24 containing 446 amino acids with the amino acid sequence as shown in SEQ ID NO: 28 in the sequence listing. The theoretical molecular weight of the protein is predicted to be 49.9 kDa by software, and the isoelectric point pI is 5.93. The amino acid sequence identity between the glycosyltransferase and the functionally identified glycosyltransferase UGTPg29 is 91%.

GT29-25: The glycosyltransferase gene GT29-25 encodes a protein GT29-25
containing 446 amino acids with the amino acid sequence as shown in SEQ ID NO: 30 in the sequence listing. The theoretical molecular weight of the protein is predicted to be 49.9 kDa by software, and the isoelectric point pI is 5.93. The amino acid sequence identity between the glycosyltransferase and the functionally identified glycosyltransferase UGTPg29 is 91%. GT29-19: The glycosyltransferase gene GT29-19 encodes a protein GT29-19 containing 442 amino acids with the amino acid sequence as shown in SEQ ID NO: 116 in the sequence listing. The theoretical molecular weight of the protein is predicted to be 49.1 kDa by software, and the isoelectric point pI is 5.47.

GT29-20: The glycosyltransferase gene GT29-20 encodes a protein GT29-20 containing 442 amino acids with the amino acid sequence as shown in SEQ ID NO: 118 in the sequence listing. The theoretical molecular weight of the protein is predicted to be 49.1 kDa by software, and the isoelectric point pI is 5.93.

GT29-21: The glycosyltransferase gene GT29-21 encodes a protein GT29-21 containing 442 amino acids with the amino acid sequence as shown in SEQ ID NO: 120 in the sequence listing. The theoretical molecular weight of the protein is predicted to be 49.1 kDa by software, and the isoelectric point pI is 5.80.

GT29-22: The glycosyltransferase gene GT29-22 encodes a protein GT29-22 containing 442 amino acids with the amino acid sequence as shown in SEQ ID NO: 122 in the sequence listing. The theoretical molecular weight of the protein is predicted to be 49.1 kDa by software, and the isoelectric point pI is 5.93.

GT29-23: The glycosyltransferase gene GT29-23 encodes a protein GT29-23 containing 442 amino acids with the amino acid sequence as shown in SEQ ID NO: 124 in the sequence listing. The theoretical molecular weight of the protein is predicted to be 49.0 kDa by software, and the isoelectric point pI is 5.61.

GT29-36: The glycosyltransferase gene GT29-36 encodes a protein GT29-36 containing 442 amino acids with the amino acid sequence as shown in SEQ ID NO:102 in the sequence listing. The theoretical molecular weight of the protein is predicted to be 49.1 kDa by software, and the isoelectric point pI is 5.93.

GT29-37: The glycosyltransferase gene GT29-37 encodes a protein GT29-37 containing 442 amino acids with the amino acid sequence as shown in SEQ ID NO: 104 in the sequence listing. The theoretical molecular weight of the protein is predicted to be 49.1 kDa by software, and the isoelectric point pI is 5.62.

GT29-42: The glycosyltransferase gene GT29-42 encodes a GT29-42 protein containing 444 amino acids with the amino acid sequence as shown in SEQ ID NO: 106 in the sequence listing. The theoretical molecular weight of the protein is predicted to be 49.4 kDa by software, and the isoelectric point pI is 6.16.

GT29-43: The glycosyltransferase gene GT29-43 encodes a protein GT29-43 containing 442 amino acids with the amino acid sequence as shown in SEQ ID NO: 108in the sequence listing. The theoretical molecular weight of the protein is predicted to be 49.1 kDa by software, and the isoelectric point pI is 5.78.

GT29-45: The glycosyltransferase gene GT29-45 encodes a protein GT29-45 containing 448 amino acids with the amino acid sequence as shown in SEQ ID NO: 110 in the sequence listing. The theoretical molecular weight of the protein is predicted to be 50.0 kDa by software, and the isoelectric point pI is 7.25.

GT29-46: The glycosyltransferase gene GT29-46 encodes a protein GT29-46 containing 442 amino acids with the amino acid sequence as shown in SEQ ID NO: 112 in the sequence listing. The theoretical molecular weight of this protein is predicted to be 49.1 kDa by software, and the isoelectric point pI is 5.48.

### Example 2

### Expression of Glycosyltransferase Genes GT29-32, GT29-33 and GT29-34 in E. coli

Using the plasmids GT29-32-pMD18T, GT29-33-pMD18T and GT29-34-pMD18T constructed in Example 1 containing GT29-32, GT29-33 and GT29-34 genes as templates, the target genes GT29-32, GT29-33 and GT29-34 were amplified with the primers as shown in Table 1.

After the expression vector pET28a (purchased from Merck) was digested with Ncol/Sall, GT29-32, GT29-33 and GT29-34 were cloned into pET28a (one-step cloning kit, purchased from Novizan) to construct E. coli expression vectors GT29-32-pET28a, GT29-33-pET28a and GT29-34-pET28a. Using the 6×His tag sequence on pET28a, the C-terminus of the recombinant proteins GT29-32, GT29-33 and GT29-34 had a 6×His tag. The plasmids were transformed into commercially available E. coli BL21 to construct recombinant strains BL21-GT29-32, BL21-GT29-33 and BL21-GT29-34. A recombinant was inoculated into LB medium, cultured at 37° C., 200 rpm to an OD600 of about 0.6-0.8, then the bacterial solution was cooled to 4° C., and IPTG with a final concentration of 100 uM was added, and the expression was induced at 18° C., 120 rpm for 16 h. The bacteria was collected by centrifugation at 4° C., and the cells were disrupted by ultrasound. The supernatant of the cell lysate was collected by centrifugation at 12000 g, at 4° C. for 10 min. The samples were taken for SDS-PAGE electrophoresis and western blot. SDS-PAGE result shows that the recombinant transformants of GT29-32-pET28a, GT29-33-pET28a and GT29-34-pET28a are not significantly different from the cell lysate of the empty vector pET28a recombinant transformant, and the soluble expression is not obvious (FIG. 1 panel A). Anti-6×His tag Western Blot (FIG. 1 panel B) shows that there is a clear band between 45 and 55 kD, and the glycosyltransferases GT29-32, GT29-33, and GT29-34 are slightly solubly expressed in E. coli.

TABLE 1
primers used to amplify genes

| gene | primer | SEQ ID NO. |
|---|---|---|
| UGT29-4 | UGT29-4-F | 31 |
| | UGT29-4-R | 34 |
| UGT29-5 | UGT29-5-F | 33 |
| | UGT29-5-R | 32 |
| UGT29-7 | UGT29-7-F | 35 |
| | UGT29-7-R | 32 |
| UGT29-9 | UGT29-9-F | 33 |
| | UGT29-9-R | 32 |
| UGT29-11 | UGT29-11-F | 33 |
| | UGT29-11-R | 32 |
| UGT29-13 | UGT29-13-F | 33 |
| | UGT29-13-R | 32 |
| UGT29-17 | UGT29-17-F | 31 |
| | UGT29-17-R | 32 |
| UGT29-18 | UGT29-18-F | 33 |
| | UGT29-18-R | 34 |
| UGT29-24 | UGT29-24-F | 33 |
| | UGT29-24-R | 34 |
| UGT29-25 | UGT29-25-F | 33 |
| | UGT29-25-R | 32 |
| UGT29-32 | UGT29-32-F | 31 |
| | UGT29-32-R | 32 |
| UGT29-33 | UGT29-33-F | 36 |
| | UGT29-33-R | 37 |
| UGT29-34 | UGT29-34-F | 36 |
| | UGT29-34-R | 34 |
| UGT29-19 | UGT29-19-F | 125 |
| | UGT29-19-R | 126 |
| UGT29-20 | UGT29-20-F | 127 |
| | UGT29-20-R | 128 |
| UGT29-21 | UGT29-21-F | 129 |
| | UGT29-21-R | 130 |
| UGT29-22 | UGT29-22-F | 131 |
| | UGT29-22-R | 132 |
| UGT29-23 | UGT29-23-F | 133 |
| | UGT29-23-R | 134 |
| UGT29-36 | UGT29-36-F | 101 |
| | UGT29-36-R | 102 |
| UGT29-37 | UGT29-37-F | 103 |
| | UGT29-37-R | 104 |
| UGT29-42 | UGT29-42-F | 105 |
| | UGT29-42-R | 106 |
| UGT29-43 | UGT29-43-F | 107 |
| | UGT29-43-R | 108 |
| UGT29-45 | UGT29-36-F | 109 |
| | UGT29-36-R | 110 |
| UGT29-46 | UGT29-36-F | 111 |
| | UGT29-36-R | 112 |

### Example 3

### In vitro Transglycosylation Activity and Product Identification of GT29-32, GT29-33 and GT29-34

The cell lysate supernatants of recombinant E. coli BL21-GT29-32, BL21-GT29-33 and BL21-GT29-34 in Example 2 was used as a crude enzyme solution to perform transglycosylation reaction, and the cell lysate of the recombinant E. coli with empty vector pET28a was used as a control.

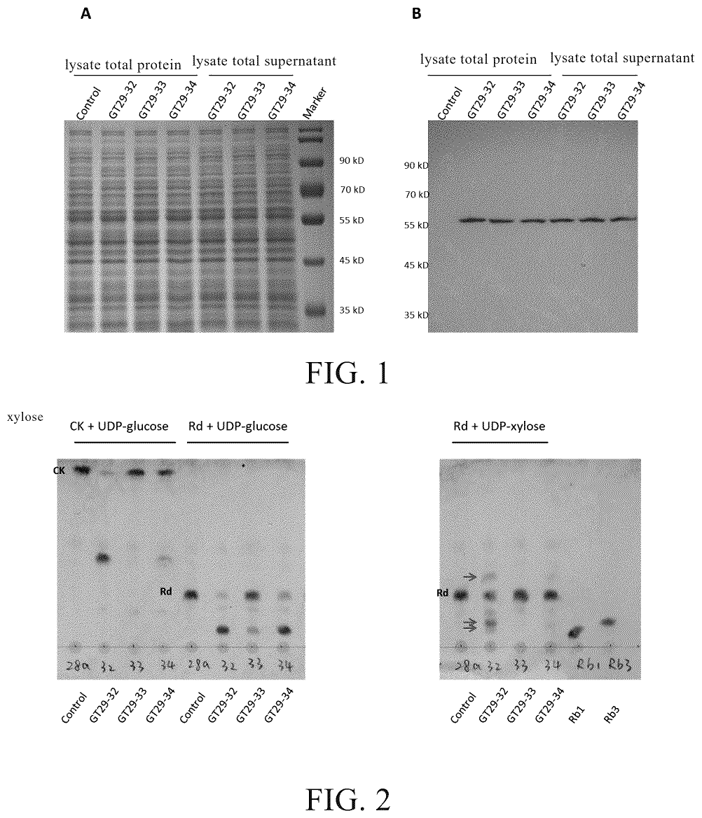

As shown in FIG. 2: using protopanaxadiol ginsenoside CK as a glycosyl receptor and UDP-glucose as a glycosyl donor, GT29-32 and GT29-34 can catalyze the formation of a new product;

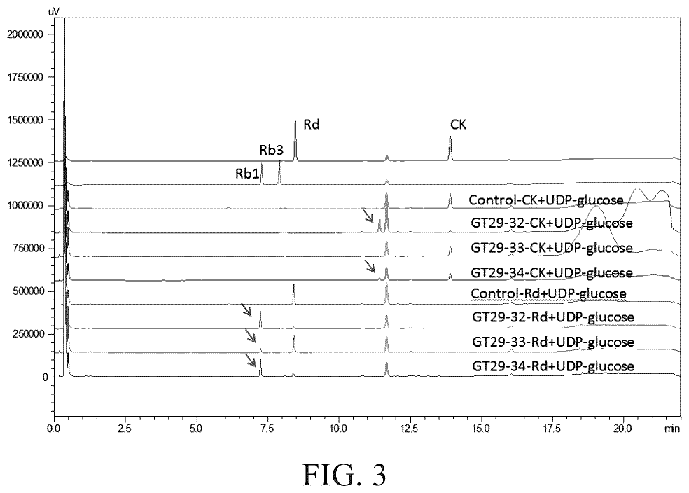

As shown in FIG. 3: using ginsenoside Rd as a glycosyl acceptor and UDP-glucose as a glycosyl donor, GT29-32, GT29-33 and GT29-34 can catalyze the formation of Rb1. The HPLC results are consistent with the TLC results.

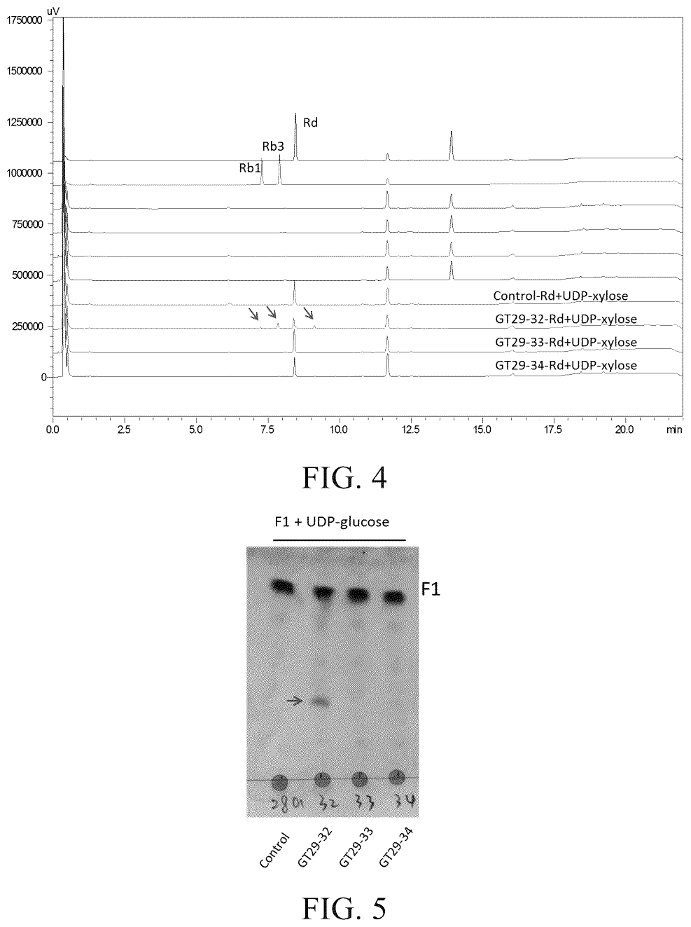

Therefore, GT29-32 and GT29-34 can catalyze the C20-O-Glc of CK extension to a molecule of glucose to generate ginsenoside Gypenoside LXXV. When UDP-xylose is used as a glycosyl donor, GT29-32 can catalyze Rd to produce three products. One of the products has the same mobility on TLC as Rb3, that is, GT29-32 can extend a molecule of xylose at C20-O-Glc to produce Rb3 (FIG. 2). The results of HPLC are consistent with those of TLC. GT29-32 catalyzes the production of three products from Rd and UDP-xylose (FIG. 4).

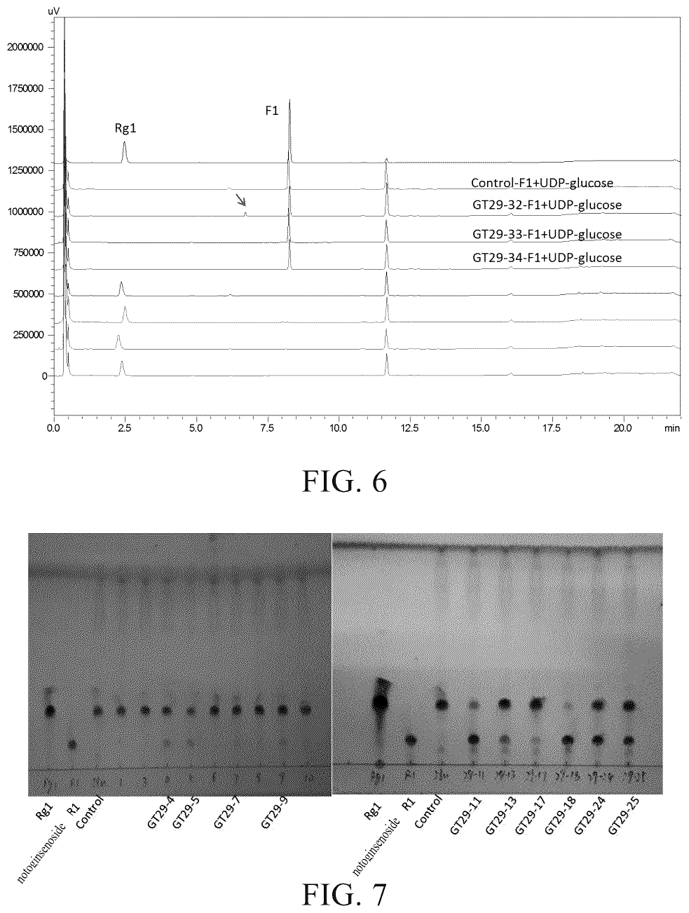

Using Protopanaxatriol Ginsenoside F1 as a glycosyl acceptor and UDP-glucose as a glycosyl donor, GT29-32 can catalyze the formation of a new product. It is speculated that it also extends a molecule of glucose at C20-O-Glc of F1, the product is Notoginsenoside R3 (FIG. 5 and FIG. 6).

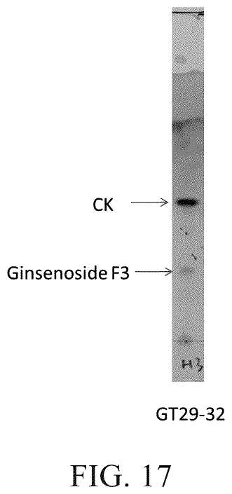

Using Protopanaxadiol Ginsenoside CK as a glycosyl acceptor and UDP-arabinose as a glycosyl donor, GT29-32, GT29-33 and GT29-34 can catalyze the first glycosyl of C-20 of CK to extend an arabinosyl to generate Ginsenoside F3, wherein GT29-32 has the strongest activity (FIG. 17).

### Example 4

### Expression of Glycosyltransferase Genes GT29-4, GT29-5, GT29-7, GT29-9, GT29-11, GT29-13, GT29-17, GT29-18, GT29-24 and GT29-25 in E. coli

Plasmids GT29-4-pMD18T, GT29-5-pMD18T, GT29-7-pMD18T, GT29-9-pMD18T, GT29-11-pMD18T, GT29-13-pMD18T, GT29-17-pMD18T, GT29-18-pMD18T, GT29-24-pMD18T and GT29-25-pMD18T containing GT29-4, GT29-5, GT29-7, GT29-9, GT29-11, GT29-13, GT29-17, GT29-18, GT29-24 and GT29-25 genes constructed in Example 1 were used as templates to amplify target genes GT29-4, GT29-5, GT29-7, GT29-9, GT29-11, GT29-13, GT29-17, GT29-18, GT29-24 and GT29-25 with the primers as shown in Table 1. After the expression vector pET28a (purchased from Merck) was digested with Ncol/Sall, GT29-11, GT29-13, GT29-17, GT29-18, GT29-24 and GT29-25 were cloned into pET28a (one-step cloning kit, purchased from Novizan), and E. coli expression vectors GT29-4-pET28a, GT29-5-pET28a, GT29-7-pET28a, GT29-9-pET28a, GT29-11-pET28a, GT29-13-pET28a, GT29-17-pET28a, GT29-18-pET28a, GT29-24-pET28a and GT29-25-pET28a were constructed.

Using the 6×His tag sequence on pET28a, recombinant proteins GT29-4-pET28a, GT29-5-pET28a, GT29-7-pET28a, GT29-9-pET28a, GT29-11-pET28a, GT29-13-pET28a, GT29-17-pET28a, GT29-18-pET28a, GT29-24 and GT29-25 had a 6×His tag at the C-terminal. The plasmids were transformed into commercially available E. coli BL21 to construct recombinant strains BL21-GT29-4, BL21-GT29-5, BL21-GT29-7, BL21-GT29-9, BL21-GT29-11, BL21-GT29-13. BL21-GT29-17, BL21-GT29-18, BL21-GT29-24 and BL21-GT29-25. A recombinant was inoculated into LB medium, cultured at 37° C., 200 rpm to an OD600 of about 0.6-0.8, then the bacterial solution was cooled to 4° C., and IPTG with a final concentration of 100 uM was added, and induced expression was performed at 18° C., 120 rpm for 16 h. The bacteria was collected by centrifugation at 4° C., and the cells were disrupted by ultrasound. The supernatant of the cell lysate was collected by centrifugation at 12000 g at 4° C. for 10 min. The samples were taken for SDS-PAGE electrophoresis and western blot.

SDS-PAGE shows recombinant transformants of GT29-4-pET28a, GT29-5-pET28a, GT29-7-pET28a, GT29-9-pET28a, GT29-11-pET28a, GT29-13-pET28a, GT29-17-pET28a, GT29-18-pET28a, GT29-24-pET28a and GT29-25-pET28a were not significantly different from the cell lysate of the empty vector pET28a recombinant transformant, and the soluble expression levels were not obvious. Anti-6×His tag Western Blot shows that there was a clear band between 45 and 55 kD, and glycosyltransferases GT29-4,

GT29-5, GT29-7, GT29-9, GT29-11, GT29-13, GT29-17, GT29-18, GT29-24 and GT29-25 had a small amount of soluble expression in E. coli .

### Example 5

### In Vitro Transglycosylation Activity and Products Identification of GT29-4, GT29-5, GT29-7, GT29-9, GT29-11, GT29-13, GT29-17, GT29-18, GT29-24 and GT29-25

The cell lysate supernatants of recombinant E. coli BL21-GT29-4, BL21-GT29-5,

BL21-GT29-7, BL21-GT29-9, BL21-GT29-11, BL21-GT29-13, BL21-GT29-17, BL21-GT29-18, BL21-GT29-24 and BL21-GT29-25 in Example 2 was used as a crude enzyme solution for transglycosylation reaction, and cell lysate of recombinant E.coli with empty vector pET28a was used as a control.

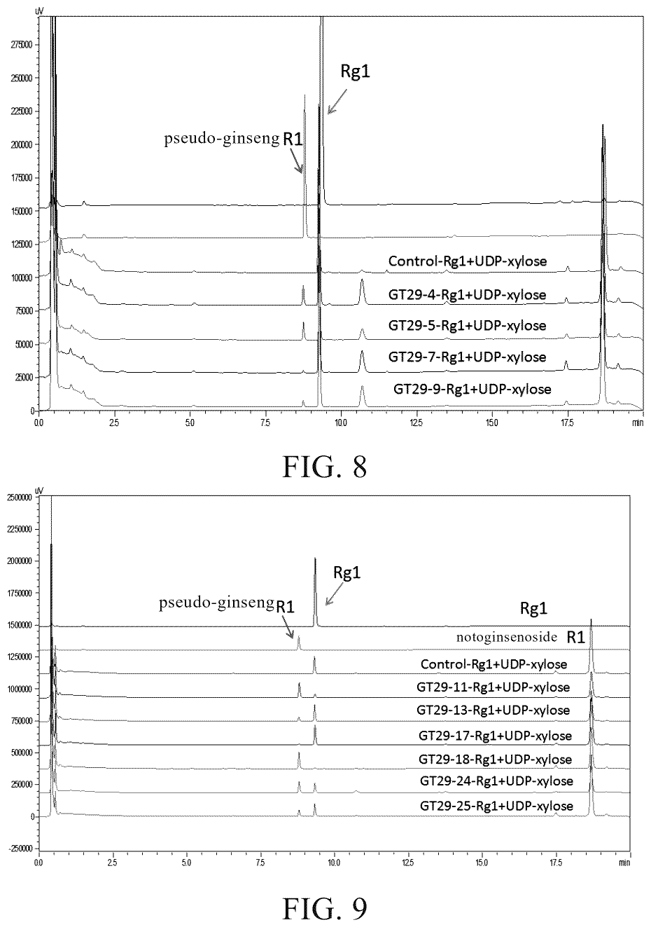

As shown in FIG. 7, using the Protopanaxadiol Ginsenoside Rgl as a glycosyl acceptor, UDP-xylose as a glycosyl donor, GT29-4, GT29-5, GT29-7, GT29-9, GT29-11, GT29-13, GT29-17, GT29-18, GT29-24 and GT29-25 can catalyze the formation of Notoginsenoside R1. The HPLC results are consistent with the TLC results (FIG. 8 and FIG. 9). Therefore, GT29-4, GT29-5, GT29-7, GT29-9, GT29-11, GT29-13, GT29-17, GT29-18, GT29-24, and GT29-25 are capable of catalyzing the extension of C6-O-Glc of Rg1 by a molecule of xylose to produce notoginsenoside R1.

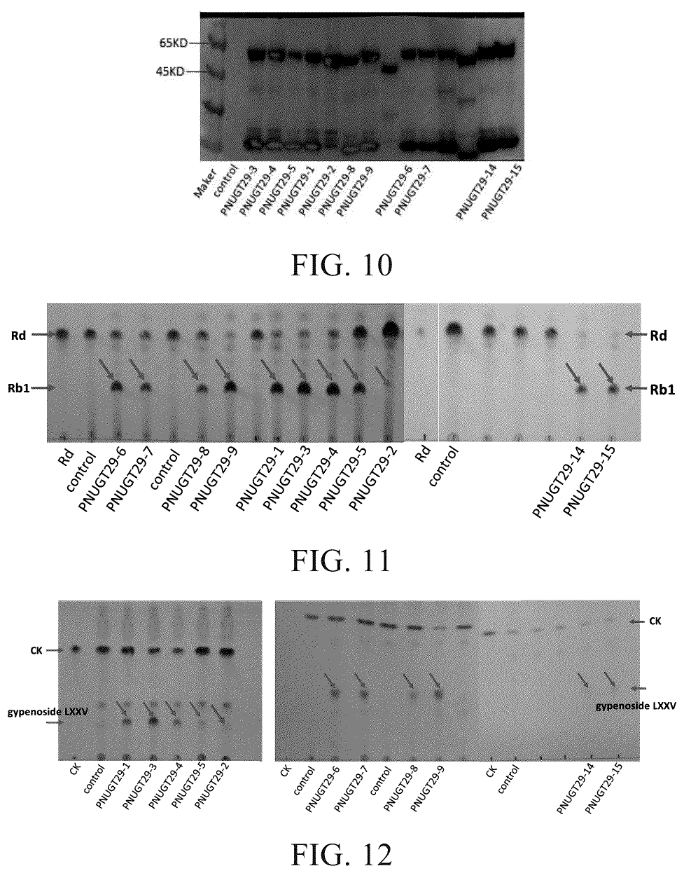

As shown in FIG. 10, GT29-24 and GT29-25 can use Protopanaxadiol Ginsenoside Rh2 as a glycosyl acceptor and UDP-glucose as a glycosyl donor to catalyze the production of ginsenoside Rg3 by extending a glucosyl at the C-3 glycosyl of Rh2. When the substrate is changed to F2, GT29-24 and GT29-25 can further catalyze the extension of a glucosyl at the C-3 glycosyl of F2 to produce ginsenoside Rd.

### Example 6

### Isolation of Panax Notoginseng Glycosyltransferase and the Coding Gene Thereof

RNA in Panax notoginseng was extracted and reverse transcription was performed to obtain cDNA of Panax notoginseng. Using this cDNA as a template, primer pair 1 (SEQ ID NO.: 82 and SEQ ID NO.: 83), primer pair 2 (SEQ ID NO.: 84 and SEQ ID NO.: 85), primer pair 3 (SEQ ID NO.: 84 and SEQ ID NO.: 86), primer pair 4 (SEQ ID NO.: 87 and SEQ ID NO.: 88) were used for PCR amplification to obtain a 1.4-1.5 kb amplification product. The high-fidelity KOD DNA polymerase from Bao Bioengineering Co., Ltd. was used as the DNA polymerase. PCR products were detected by agarose gel electrophoresis.

According to Example 1, several clones were selected to extract recombinant plasmids and sequenced to obtain 14 different nucleic acid sequences, named PNUGT29-1 (SEQ ID NO.: 38), PNUGT29-2 (SEQ ID NO.: 40), PNUGT29-3 (SEQ ID NO.: 42), PNUGT29-4 (SEQ ID NO.: 44), PNUGT29-5 (SEQ ID NO.: 46), PNUGT29-6 (SEQ ID NO.: 48), PNUGT29-7 (SEQ ID NO.: 50), PNUGT29-8 (SEQ ID NO.: 52), PNUGT29-9 (SEQ ID NO.: 54), PNUGT29-14 (SEQ ID NO.: 56) and PNUGT29-15 (SEQ ID NO.: 58), respectively. BESTORF software was used to find ORF. Through sequence alignment, the amplification products all have the conserved functional domain of glycosyltransferase family 1, indicating that it is a glycosyltransferase gene.

PNUGT29-1: The glycosyltransferase gene PNUGT29-1 encodes a protein PNUGT29-1 containing 447 amino acids with the amino acid sequence as shown in SEQ ID NO: 39 in the sequence listing. The theoretical molecular weight of the protein is predicted to be 49.688 kDa by software, and the isoelectric point pI is 6.58.

PNUGT29-2: The glycosyltransferase gene PNUGT29-2 encodes a protein PNUGT29-2 containing 442 amino acids with the amino acid sequence as shown in SEQ ID NO: 41 in the sequence listing. The theoretical molecular weight of the protein is predicted to be 49.118 kDa by software, and the isoelectric point pI is 6.20.

PNUGT29-3: The glycosyltransferase gene PNUGT29-3 encodes a protein PNUGT29-3 containing 447 amino acids with the amino acid sequence as shown in SEQ ID NO: 43 in the sequence listing. The theoretical molecular weight of the protein is predicted to be 49.729 kDa by software, and the isoelectric point pI is 6.58.

PNUGT29-4: The glycosyltransferase gene PNUGT29-4 encodes a protein PNUGT29-4 containing 447 amino acids with the amino acid sequence as shown in SEQ ID NO: 45 in the sequence listing. The theoretical molecular weight of the protein is predicted to be 49.715 kDa by software, and the isoelectric point pI is 6.58.

PNUGT29-5: The glycosyltransferase gene PNUGT29-5 encodes a protein PNUGT29-5 containing 447 amino acids with the amino acid sequence as shown in SEQ ID NO: 47 in the sequence listing. The theoretical molecular weight of the protein is predicted to be 49.718 kDa by software, and the isoelectric point pI is 6.45.

PNUGT29-6: The glycosyltransferase gene PNUGT29-6 encodes a protein PNUGT29-6 containing 447 amino acids with the amino acid sequence as shown in SEQ ID NO: 49 in the sequence listing. The theoretical molecular weight of the protein is predicted to be 49.657 kDa by software, and the isoelectric point pI is 6.70.

PNUGT29-7: The glycosyltransferase gene PNUGT29-7 encodes a protein. PNUGT29-7 containing 447 amino acids with the amino acid sequence as shown in SEQ ID NO: 51 in the sequence listing. The theoretical molecular weight of the protein is predicted to be 49.749 kDa by software, and the isoelectric point pI is 6.58.

PNUGT29-8: The glycosyltransferase gene PNUGT29-8 encodes a protein. PNUGT29-8 containing 447 amino acids with the amino acid sequence as shown in SEQ ID NO: 53 in the sequence listing. The theoretical molecular weight of the protein is predicted to be 49.657 kDa by software, and the isoelectric point pI is 6.70.

PNUGT29-9: The glycosyltransferase gene PNUGT29-9 encodes a protein PNUGT29-9 containing 447 amino acids with the amino acid sequence as shown in SEQ ID NO: 55 in the sequence listing. The theoretical molecular weight of the protein is predicted to be 49.695 kDa by software, and the isoelectric point pI is 6.58.

PNUGT29-14: The glycosyltransferase gene PNUGT29-14 encodes a protein PNUGT29-14 containing 447 amino acids with the amino acid sequence as shown in SEQ ID NO: 57 in the sequence listing. The theoretical molecular weight of the protein is predicted to be 49.778 kDa by software, and the isoelectric point pI is 6.70. PNUGT29-15: The glycosyltransferase gene.

PNUGT29-15 encodes a protein PNUGT29-15 containing 447 amino acids with the amino acid sequence as shown in SEQ ID NO: 59 in the sequence listing. The theoretical molecular weight of the protein is predicted to be 49.755 kDa by software, and the isoelectric point p1 is 6.63.

### Example 7

### Expression of Panax notoginseng glycosyltransferase genes PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 and NUGT29-15 in E. coli

Plasmids PNUGT29-1-pMD18T, PNUGT29-2-pMD18T, PNUGT29-3-pMD18T, PNUGT29-4-pMD18T, PNUGT29-5-pMD18T, PNUGT29-6-pMD18T, PNUGT29-7-pMD18T, PNUGT29-8-pMD18T, PNUGT29-9-pMD18T, PNUGT29-14-pMD18T and PNUGT29-15-pMD18T containing PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 and PNUGT29-15 genes constructed in Example 6 were used as a template, and the target genes PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 and PNUGT29-15 were amplified with the primers as shown in Table 1. Referring to the method in Example 2, the recombinant strains BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7. BL21-PNUGT29-8, BL21-PNUGT29-9, BL21-PNUGT29-14 and BL21-PNUGT29-15 were constructed for SDS-PAGE electrophoresis and western blot. Anti-6 x His tag Western Blot (FIG. 10) shows that there is a clear band between 45 and 65 kD, glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 and PNUGT29-15 have a small amount of soluble expression in E. coli.

TABLE 2
primers used to amplify genes

| | | SEQ ID |
|---|---|---|
| gene | primer | NO. |
| PNUGT29-1 | PNUGT29-1-F | 60 |
| | PNUGT29-1-R | 61 |
| PNUGT29-2 | PNUGT29-2-F | 62 |
| | PNUGT29-2-R | 63 |
| PNUGT29-3 | PNUGT29-3-F | 64 |
| | PNUGT29-3-R | 65 |
| PNUGT29-4 | PNUGT29-4-F | 66 |
| | PNUGT29-4-R | 67 |
| PNUGT29-5 | PNUGT29-5-F | 68 |
| | PNUGT29-5-R | 69 |
| PNUGT29-6 | PNUGT29-6-F | 70 |
| | PNUGT29-6-R | 71 |
| PNUGT29-7 | PNUGT29-7-F | 72 |
| | PNUGT29-7-R | 73 |
| PNUGT29-8 | PNUGT29-8-F | 74 |
| | PNUGT29-8-R | 75 |
| PNUGT29-9 | PNUGT29-9-F | 76 |
| | PNUGT29-9-R | 77 |
| PNUGT29-14 | PNUGT29-14-F | 78 |
| | PNUGT29-14-R | 79 |
| PNUGT29-15 | PNUGT29-15-F | 80 |
| | PNUGT29-15-R | 81 |

### Example 8

### In Vitro Transglycosylation Activity And Product Identification of Panax

Notoginseng Glycosyltransferases PNUGT29-1, PNUGT29-2, PNUGT29-3, PNUGT29-4, PNUGT29-5, PNUGT29-6, PNUGT29-7, PNUGT29-8, PNUGT29-9, PNUGT29-14 and PNUGT29-15

The cell lysate supernatants of recombinant E. coli BL21-PNUGT29-1, BL21-PNUGT29-2, BL21-PNUGT29-3, BL21-PNUGT29-4, BL21-PNUGT29-5, BL21-PNUGT29-6, BL21-PNUGT29-7, BL21-PNUGT29-8, BL21-PNUGT29-9, BL21-PNUGT29-14 and BL21-PNUGT29-15 in Example 7 were used as a crude enzyme solution for transglycosylation reaction. Cell lysate of recombinant E.coli with empty vector pET28a was used as a control.

As shown in FIG. 11: using Protopanaxadiol Ginsenoside Rd as a glycosyl acceptor, UDP-glucose as a glycosyl donor, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 can catalyze the extension of a glucosyl at the C-20 glycosyl of Rd to generate Rb1.

As shown in FIG. 12: using Protopanaxadiol Ginsenoside CK as a glycosyl acceptor, UDP-glucose as a glycosyl donor, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 can catalyze the extension of a glucosyl at the C-20 glycosyl to generate Gypenoside LXXV.

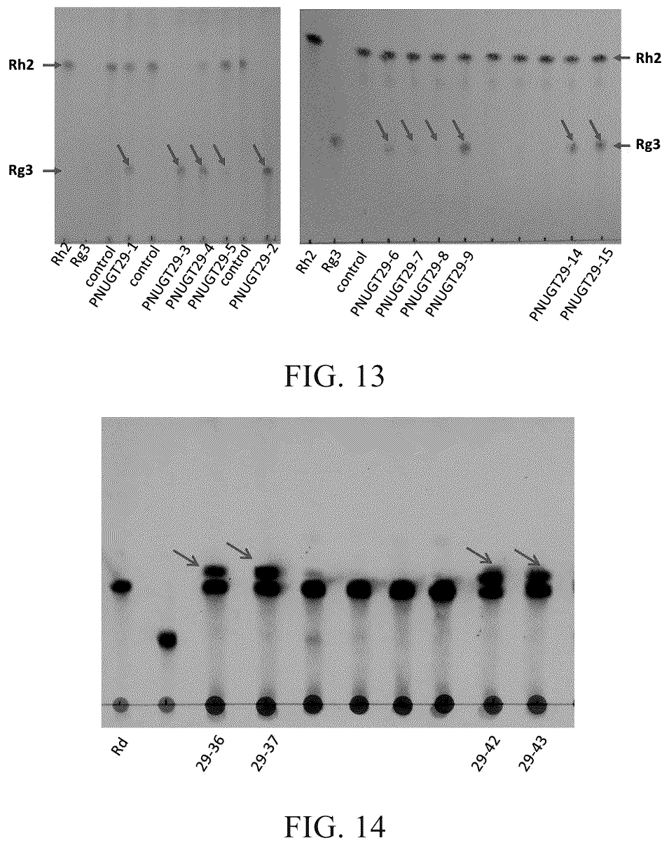

As shown in FIG. 13: using Protopanaxadiol Ginsenoside Rh2 as a glycosyl acceptor, UDP-glucose as a glycosyl donor, PNUGT29-1, PNUGT29-2, PNUGT29-3, PNUGT29-4, PNUGT29-5, PNUGT29-6, PNUGT29-7, PNUGT29-8, PNUGT29-9, PNUGT29-14, PNUGT29-15 can catalyze the extension of a glucosyl at the C-3 glycosyl of Rh2to generate Rg3.

### Example 9

### Expression of Glycosyltransferase genes GT29-19, GT29-20, GT29-21, GT29-22, GT29-23, GT29-36, GT29-37, GT29-42, GT29-43, GT29-45 and GT29-46 in E. coli

Plasmids GT29-19-pMD18T, GT29-20-pMD18T, GT29-21-pMD18T, GT29-22-pMD18T, GT29-23-pMD18T, GT29-36-pMD18T, GT29-37-pMD18T, GT29-42-pMD18T, GT29-43-pMD18T, GT29-45-pMD18T, and GT29-46-pMD18T containing GT29-19, GT29-20, GT29-21, GT29-22, GT29-23, GT29-36, GT29-37, GT29-42, GT29-43, GT29-45 and GT29-46 genes constructed in Example 1 were used as a template, and the target genes GT29-36, GT29-37, GT29-42, GT29-43, GT29-45 and GT29-46 were amplified with the primers as shown in Table 1.

Referring to Example 2, recombinant strains BL21-GT29-19, BL21-GT29-20, BL21-GT29-21, BL21-GT29-22, BL21-GT29-23, BL21-GT29-36, BL21-GT29-37, BL21-GT29-42, BL21-GT29-43, BL21-GT29-45 and BL21-GT29-46 were constructed, and samples were taken for SDS-PAGE electrophoresis and western blot.

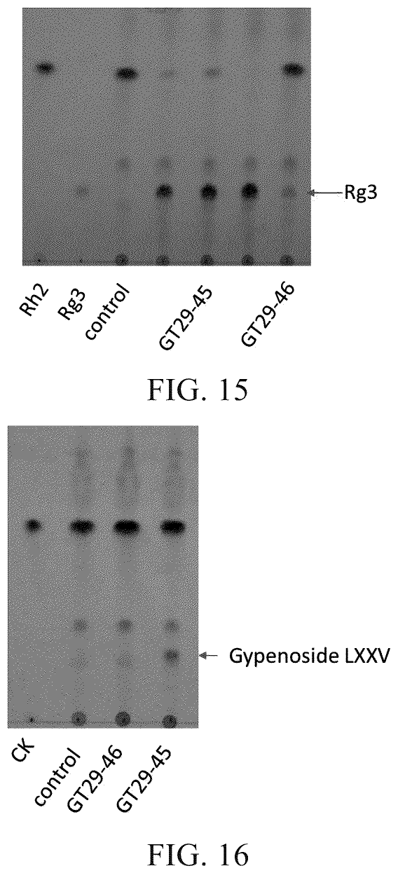

Protopanaxadiol Ginsenoside Rh2 was used as a glycosyl acceptor, and UDP-glucose was used as a glycosyl donor, and the above-mentioned glycosyltransferases GT29-19, GT29-20, GT29-21, GT29-22, GT29-23, GT29-36, GT29-37, GT29-42, GT29-43, GT29-45 and GT29-46 can all catalyze the extension of a glucosyl at the C-3glycosyl of Rh2 to generate Rg3. FIG. 15 shows GT29-45 and GT29-46 can catalyze Rh2 to generate Rg3.

Protopanaxadiol Ginsenoside Rd was used as a glycosyl acceptor and UDP-xylose was used as a glycosyl donor, and the above glycosyltransferases GT29-19, GT29-20, GT29-21, GT29-22, GT29-23, GT29-36, GT29-37, GT29-42, GT29-43 can all catalyze the replacement of the second glucose at C-3 position of Rd with xylose to produce a new triterpene saponin (3-O-β-(D-xylopyranosyl)-β-(D-glucopyranosyl), 20-O-β-(D-glucopyranosyl)-PPD), of which GT29-36, GT29-37, GT29-42 and GT29-43 are the most active (FIG. 14).

As shown in FIG. 16: Protopanaxadiol Ginsenoside CK is used as a glycosyl acceptor, and UDP-glucose is used as a glycosyl donor. GT29-45 and GT29-46 can catalyze the C-20 glycosyl of CK to extend a glucosyl to produce Gypenoside LXXV, in which GT29-45 has a strong activity.

### Example 10

### Further Verification of the Glycosyltransferase Activity

The above Examples 3, 5, and 8 were repeated, and the difference was that other glycosyl donors and substrates were replaced, and the experimental results are shown in Table 3 to Table 5:

TABLE 3

| | | C-3 | | |
|---|---|---|---|---|
| | | UDP-xylose | UDP-G | |
| SEQ ID NO: | | Rd | F2 | Rh2 |
| 116 | GT29-19 | ++ | ++ | +++ |
| 118 | GT29-20 | ++ | ++ | +++ |
| 120 | GT29-21 | + | ++ | +++ |
| 122 | GT29-22 | + | ++ | ++ |
| 124 | GT29-23 | + | ++ | ++ |
| 90 | GT29-36 | +++ | ++ | ++ |
| 92 | GT29-37 | +++ | ++ | ++ |
| 94 | GT29-42 | +++ | ++ | ++ |
| 96 | GT29-43 | +++ | ++ | ++ |
| 98 | GT29-45 | NS | ++ | ++ |
| 100 | GT29-46 | NS | ++ | ++ |
| 39 | PNUGT29-1 | NS | ++ | +++ |
| 41 | PNUGT29-2 | NS | +++ | +++ |
| 43 | PNUGT29-3 | NS | +++ | +++ |
| 45 | PNUGT29-4 | NS | +++ | +++ |
| 47 | PNUGT29-5 | NS | ++ | ++ |
| 49 | PNUGT29-6 | NS | ++ | ++ |
| 51 | PNUGT29-7 | NS | ++ | ++ |
| 53 | PNUGT29-8 | NS | ++ | ++ |
| 55 | PNUGT29-9 | NS | +++ | +++ |
| 57 | PNUGT29-14 | NS | ++ | ++ |
| 59 | PNUGT29-15 | NS | ++ | ++ |

TABLE 4

| | | C-6 | | | |
|---|---|---|---|---|---|
| | | UDP-xylose | | UDP-G | |
| SEQ ID NO: | name | Rg1 | Rhl | Rg1 | Rhl |
| 12 | GT29-4 | ++ | ++ | + | + |
| 14 | GT29-5 | ++ | ++ | + | + |
| 16 | GT29-7 | +++ | ++ | ++ | ++ |
| 18 | GT29-9 | ++ | ++ | + | + |
| 20 | GT29-11 | +++ | +++ | ++ | ++ |
| 22 | GT29-13 | ++ | ++ | ++ | ++ |
| 24 | GT29-17 | ++ | ++ | + | + |
| 26 | GT29-18 | +++ | +++ | ++ | ++ |
| 28 | GT29-24 | +++ | +++ | ++ | ++ |
| 30 | GT29-25 | +++ | +++ | + | + |

TABLE 5

| | | C-20 | | | | |
|---|---|---|---|---|---|---|
| | | | | | | UDP- |
| | | UDP-xylose | UDP-G | | | arabinose |
| SEQ ID NO: | name | Rd | CK | F1 | Rd | CK |
| 4 | GT29-32 | +++ | +++ | ++ | + | ++ |
| 6 | GT29-33 | + | ++ | + | ++ | ++ |
| 8 | GT29-34 | + | ++ | + | ++ | ++ |
| 98 | GT29-45 | + | ++ | + | + | NS |
| 100 | GT29-46 | + | + | + | + | NS |
| 39 | PNUGT29-1 | + | +++ | + | + | NS |
| 41 | PNUGT29-2 | + | + | + | + | NS |
| 43 | PNUGT29-3 | + | +++ | ++ | + | NS |
| 45 | PNUGT29-4 | + | +++ | ++ | ++ | NS |
| 47 | PNUGT29-5 | + | +++ | + | ++ | NS |
| 49 | PNUGT29-6 | + | +++ | ++ | ++ | NS |
| 51 | PNUGT29-7 | + | +++ | + | + | NS |
| 53 | PNUGT29-8 | + | +++ | + | + | NS |
| 55 | PNUGT29-9 | + | +++ | + | + | NS |
| 57 | PNUGT29-14 | + | +++ | + | + | NS |
| 59 | PNUGT29-15 | + | +++ ++ | + | + | NS |

* NS stands for not shown

It can be seen from Tables 3 to 5 that the glycosyltransferases of the present invention can utilize common glycosyl donors and substrates, and have glycosyl extension or glycosyl substitution activity on different sites of tetracyclic triterpenes.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. An in vitro glycosylation method, comprising the steps of:
(i) transforming or transducing a host cell with a polynucleotide encoding a glycosyltransferase, or with a recombinant expression vector containing the polynucleotide;
(ii) culturing the host cell in a culture medium, thereby expressing the glycosyltransferase;
(iii) isolating and purifying the glycosyltransferase from the culture medium or the host cell;
and (iv) transferring a glycosyl group from a glycosyl donor to the following positions of the tetracyclic triterpenoid in the presence of a glycosyltransferase:
the first glycosyl group on position C6;
thereby forming a glycosylated tetracyclic triterpenoid;
wherein the glycosyltransferase is selected from the group consisting of:
a glycosyltransferase as shown in SEQ ID NO: 28, 30, 12, 14, 16, 18, 20, 22, 24, and 26 or a derivative polypeptide thereof.

2. The method of claim 1, wherein the derivative polypeptide is independently selected from the group consisting of:
(a) a polypeptide having any of the amino acid sequence as shown in SEQ ID NOs.: 28, 30,12, 14, 16, 18, 20, 22, 24, and 26;
(b) a derivative polypeptide formed by adding a tag sequence, a signal sequence or a secretion signal sequence to SEQ ID NOs: 28, 30, 12, 14, 16, 18, 20, 22, 24, or 26 and having glycosyltransferase activity;
(c) a derivative polypeptide of an amino acid sequence having an identity of >95% with the amino acid sequence of any of SEQ ID NOs: 28, 30, 12, 14, 16, 18, 20, 22, 24, and 26 and having glycosyltransferase activity.

3. An isolated polypeptide, wherein the isolated polypeptide is:
a polypeptide or a derivative polypeptide thereof having an amino acid sequence as shown in SEQ ID NO: 28 or 30; wherein the derivative polypeptide is selected from the group consisting of:
(a) a polypeptide having an amino acid sequence as shown in SEQ ID NO: 28 or 30;
(b) a derivative polypeptide formed by adding a tag sequence, a signal sequence or a secretion signal sequence to SEQ ID NO: 28 or 30 and having a glycosyltransferase activity;
(c) a derivative polypeptide of an amino acid sequence having an identity of >95% with the amino acid sequence as shown in SEQ ID NO: 28 or 30 and having a glycosyltransferase activity.

4. An isolated polynucleotide, wherein the polynucleotide is selected from the group consisting of:
(A) a nucleotide sequence encoding the polypeptide of claim 3;
(B) a nucleotide sequence encoding a polypeptide as shown in SEQ ID NO: 28 or 30;
(C) a nucleotide sequence as shown in SEQ ID NO: 27 or 29;
(D) a nucleotide sequence having an identity of >95% (preferably >98%) with a nucleotide sequence as shown in SEQ ID NO: 27 or 29; and
(E) a nucleotide sequence that is complementary (preferably completely complementary) to the nucleotide sequence of any of (A)-(D).

5. A vector comprising the polynucleotide of claim 4.

6. A method for catalyzing one or more glycosyl group transferring reactions, comprising:
(i) transforming or transducing a host cell with a polynucleotide encoding a glycosyltransferase, or with a recombinant expression vector containing the polynucleotide;
(ii) culturing the host cell in a culture medium, thereby expressing the glycosyltransferase;
(iii) isolating and purifying the glycosyltransferase from the culture medium or the host cell; and
(iv) transferring the glycosyl group from the glycosyl donor to the first glycosyl group on position C6 of the tetracyclic triterpenoid to extend the carbohydrate chain;
wherein the glycosyltransferase is selected from the group consisting of:
a glycosyltransferase as shown in SEQ ID NO: 28, 30, 12, 14, 16, 18, 20, 22, 24, and 26 or a derivative polypeptide thereof.

7. The method of claim 6, wherein the derivative polypeptide is independently selected from the group consisting of:
(a) a polypeptide having any of the amino acid sequence as shown in SEQ ID NOs: 28, 30, 12, 14, 16, 18, 20, 22, 24, and 26;
(b) a derivative polypeptide formed by adding a tag sequence, a signal sequence or a secretion signal sequence to SEQ ID NOs: 28, 30, 12, 14, 16, 18, 20, 22, 24, or 26 and having glycosyltransferase activity;
(c) a derivative polypeptide of an amino acid sequence having an identity of >95% with the amino acid sequence of any of SEQ ID NOs: 28, 30, 12, 14, 16, 18, 20, 22, 24, and 26 and having glycosyltransferase activity.

8. A genetically engineered host cell containing the vector of claim 5.

9. A method for preparing an enzyme catalytic reagent, or for producing a glycosyltransferase, or as a catalytic cell, or producing Formula (II), (IV), (VI), (VIII) or (X) compounds, comprising culturing the host cell of claim 7 to harvest the host cell and culture medium thereof.

10. A method for producing a transgenic plant, comprising the steps of: regenerating the genetically engineered host cell of claim 7 into a plant, and the genetically engineered host cell is a plant cell.
